SEQUENCE LISTING (9 sequences)

SEQ ID NO: 1 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - protein, 497 aa, Artificial sequence
  Human glucocerebrosidase H145L/K321N variant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLLNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 4 - protein, 497 aa, Artificial sequence
  Human glucocerebrosidase H145L variant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLLNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 5 - protein, 497 aa, Artificial sequence
  Human glucocerebrosidase K321N variant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - protein, 17 aa, Yarrowia lipolytica
MKLSTILFTACATLAAA

SEQ ID NO: 7 - protein, 522 aa, Artificial sequence
  Human glucocerebrosidase H145L/K321N variant - His8 polypeptide construct
MKLSTILFTACATLAAAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLLNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQHHHHHHHH

SEQ ID NO: 8 - protein, 514 aa, Artificial sequence
  Human glucocerebrosidase H145L/K321N variant polypeptide construct
MKLSTILFTACATLAAAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLLNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - DNA, 1569 bp, Artificial sequence
  Codon optimized sequence encoding human glucocerebrosidase H145L/K321N variant - His8 polypeptide construct
atgaagctgtccaccattctcttcaccgcctgtgctaccctcgccgccgctgctcgaccatgcatccccaagtccttcggctactcctctgtcgtgtgtgtctgcaacgctacctactgtgactctttcgacccgcccaccttccccgctctgggcaccttctcccgatacgagtctacccgatctggacgacgaatggagctctctatgggtcccattcaggctaaccacaccggtaccggactgctcctcaccctgcagcccgagcagaagttccagaaggtgaagggtttcggtggagctatgaccgacgctgctgccctcaacatcctggctctctctcccccggctcagaacctcctgctgaagtcctacttctctgaggaaggtattggctacaacatcattcgagtgcccatggcctcctgcgacttctctatccgaacctacacctacgccgacacccccgacgacttccagctgctcaacttctctctccccgaggaagacaccaagctgaagattcccctcattcaccgagctctccagctggctcagcgacccgtgtctctcctggcttctccctggacctctcccacctggctcaagaccaacggtgccgtcaacggcaagggatctctgaagggccagcccggagacatctaccaccagacctgggctcgatacttcgtgaagttcctcgacgcctacgctgagcacaagctgcagttctgggctgtcaccgccgagaacgagccctctgccggactgctctccggttaccccttccagtgtctcggtttcacccccgagcaccagcgagacttcattgcccgagacctcggtcccaccctcgccaactccacccaccacaacgtccgactgctgatgctcgacgaccagcgactcctcctgccccactgggccaaggtggtcctgaccgaccccgaggccgctaagtacgtgcacggcattgctgtgcactggtacctggacttcctcgctcccgccaacgctaccctcggcgagacccaccgactgttccccaacaccatgctgttcgcctctgaggcttgcgtgggttccaagttctgggagcagtccgtgcgactgggttcctgggaccgaggaatgcagtactctcactctattatcaccaacctgctgtaccacgtcgtgggttggaccgactggaacctcgctctcaaccccgagggtggacccaactgggtccgaaacttcgtcgactctcccattatcgtcgacatcaccaaggacaccttctacaagcagcccatgttctaccacctgggacacttctctaagttcattcccgagggctcccagcgagtgggactggtggcttctcagaagaacgacctcgacgctgtcgccctgatgcaccccgacggctctgccgtcgtggtcgtcctcaaccgatcctctaaggacgtccccctcaccattaaggaccccgctgtcggtttcctggagaccatctctcccggttactctatccacacctacctctggcgacgacagcaccaccaccaccaccaccaccactaa

(19) United States
(12) Patent Application Publication
(10) Pub. No.: US 2022/0125892 A1
(43) Pub. Date: Apr. 28, 2022

(54) GLUCOCEREBROSIDASE POLYPEPTIDES

(71) Applicant: OXYRANE UK LTD.

(72) Inventors: Wouter Vervecken, Landskouter (BE); Steven Geysens, Wannegem-Lede (BE)

(21) Appl. No.: 17/427,308

(22) PCT Filed: Jan. 31, 2020
(86) PCT No.: PCT/EP2020/052377

§ 371 Date: Jul. 30, 2021

(30) Foreign Application Priority Data
Feb. 1, 2019 (EP) 19155111.8

(51) Int. Cl. A61K 38/47; C12N 9/24

(52) U.S. Cl. CPC A61K 38/47; C12Y 302/01045; C12N 9/2402

(57) ABSTRACT

The present invention provides glucocerebrosidase preparations, uses thereof as well as methods employing such, particularly in therapy of conditions involving glucocerebrosidase deficiency, such as Gaucher disease and glucocerebrosidase-associated alpha-synucleinopathies.

### FIELD

The invention is broadly in the field of enzyme replacement therapy (ERT), more precisely in the field of polypeptide products for use in the treatment of Lysosomal Storage Diseases (LSDs). In particular, the invention concerns glucocerebrosidase (GCase) polypeptides, and related products, uses and methods.

### BACKGROUND

Lysosomal Storage Diseases (LSDs) are a diverse group of hereditary metabolic disorders characterized by the accumulation of storage products in the lysosomes due to impaired activity of catabolic enzymes involved in their degradation. The build-up of storage products leads to cell dysfunction and progressive clinical manifestations. Deficiencies in lysosomal enzyme activities, particularly in lysosomal hydrolase activities, can be corrected by enzyme replacement therapy (ERT), provided that the administered enzyme can be effectively targeted to the lysosomes of the diseased cells. At present, ERT is the preferred path of intervention to treat LSDs, in particular systemic LSDs.

Glucocerebrosidase (D-glucocerebrosidase, GCase, GC, lysosomal acid glucosylceramidase) is a soluble lysosomal enzyme needed for the hydrolysis of glycolipids such as glucosylceramide (GlcCer) and glucosylsphingosine (GlcSph). Gaucher disease is a lysosomal storage disease caused by mutations in the gene encoding glucocerebrosidase, resulting in toxic accumulation of the enzyme's substrates in the lysosomes of certain cell types, predominantly macrophages, while other cell types can be affected as well. This metabolic disorder presents as a multi-system disease characterised by several clinical symptoms such as anaemia, thrombocytopenia, hepatosplenomegaly, bone pathology and in some cases neurological symptoms.

Three different forms of Gaucher disease have been clinically well described. The most prevalent form is the so-called non-neuronopathic form (type 1 GD, GD1), which is essentially a macrophage disorder lacking primary CNS involvement. Patients with type 1 GD can display a large variety of somatic symptoms, ranging from almost asymptomatic to those who display childhood onset disease (Charrow et al. The Gaucher registry: demographics and disease characteristics of 1698 patients with Gaucher disease. Arch Intern Med. 2000, vol. 160, 2835-43). A small number of patients is characterized by lung involvement, including interstitial lung disease and pulmonary hypertension (Mistry et al. Pulmonary hypertension in type 1 Gaucher's disease: genetic and epigenetic determinants of phenotype and response to therapy. Mol Genet Metab. 2002, vol. 77, 91-8). Type 2 GD is an acute neuronopathic form with an onset of symptoms before the age of two years and a fast progression of the disease manifestation. It is characterised by severe neurological impairments, starting with oculomotor abnormalities and followed by limited psychomotor development. Death usually follows within the first two years of the onset of the disorder. Type 3 GD is the subacute or chronic neuronopathic variant, characterised by various degrees of both systemic and neurological involvement. The latter usually appears later in life compared to the Type 2 form and includes abnormal eye movement, seizures and dementia. Patients can survive until the third or fourth decade of life (Kraoua et al. A French experience of type 3 Gaucher disease: Phenotypic diversity and neurological outcome of 10 patients. Brain Dev. 2011, vol. 33, 131-9). It is generally accepted that manifestations of pathology in neuronopathic Gaucher disease (nGD) is in part due to substrate accumulation and subsequent dysfunction in neuronal cells (Korkotian et al. Elevation of intracellular glucosylceramide levels results in an increase in endoplasmic reticulum density and in functional calcium stores in cultured neurons. J Biol Chem. 1999, vol. 274, 21673-8) (Pelled et al. The increased sensitivity of neurons with elevated glucocerebroside to neurotoxic agents can be reversed by imiglucerase. J Inherit Metab Dis. 2000, vol. 23, 175-84). While nGD patients are characterised by pathological symptoms in the brain, they also have peripheral manifestations of the disease.

Recently, a link between GCase deficiency and alpha-synuclein aggregation has also emerged, identifying glucocerebrosidase-associated alpha-synucleinopathies including inter alia parkinsonism and Parkinson's disease as an important group of neurological disorders, and glucocerebrosidase-based therapies as a potentially promising treatment strategy (Murphy et al. Glucocerebrosidase deficits in sporadic Parkinson disease. Autophagy 2014, vol. 10, 1350-1; O'Regan et al. Glucocerebrosidase Mutations in Parkinson Disease. J Parkinsons Dis. 2017, vol. 7, 411-422; Rockenstein et al. Glucocerebrosidase modulates cognitive and motor activities in murine models of Parkinson's disease. Hum Mol Genet. 2016, vol. 25, 2645-60; Sardi et al. Augmenting CNS glucocerebrosidase activity as a therapeutic strategy for parkinsonism and other Gaucher-related synucleinopathies. Proc Natl Acad Sci USA. 2013, vol. 110, 3537-42).

Currently there is no cure for Gaucher disease. However, enzyme replacement therapy (ERT) in which intravenously (IV) administered recombinant GCase is partially supplementing the deficient enzyme, is an approved treatment to alleviate the symptoms of type 1 GD. In particular, three different enzyme preparations, based on the recombinant expression of human GCase possessing N-glycans with terminal mannose residues to improve mannose receptor-mediated uptake in macrophages, imiglucerase (Cerezyme®), velaglucerase alpha (VPRIV®), and taliglucerase alfa (Elelyso®), have been approved as ERTs to manage type 1 GD.

These enzyme preparations are not used for the treatment of the neuronopathic forms of Gaucher disease, since they are unable to cross the blood-brain barrier (BBB). Moreover, pre-clinical studies on direct delivery of such enzymes into the brain of diseased mice showed only limited success (Cabrera-Salazar et al. Intracerebroventricular delivery of glucocerebrosidase reduces substrates and increases lifespan in a mouse model of neuronopathic Gaucher disease. Exp Neurol. 2010, vol. 225, 436-44). There thus exist no currently available treatment options for the neuronopathic Gaucher types 2 and 3.

Further, U.S. Pat. No. 8,962,564 discloses variant human GCase proteins having variation(s) at amino acid positions F316, L317, K321 or H145, aiming to improve the stability of human GCase and thereby increase the retention of enzymatic activity under conditions of neutral pH and body temperature. The authors proposed that variations at position(s) F316 or L317 would form a better ordered conformation near the active site, less prone to unwanted destabilization under physiological conditions; the variation K321N would stabilize an α-helix near the active site, which would result in a more open and active conformation of the catalytic site; and the variation H145L in a random coil region not in proximity of the catalytic site would facilitate better interactions between amino acid residues of adjacent secondary structures.

WO 03/056897 teaches a method for preparing phosphorylated GCase, in which GCase is enzymatically treated with isolated N-acetylglycosamine (GlcNAc) phosphotransferase, which catalyses the transfer of GlcNAc-1-phosphate from UDP-GlcNAc to the 6 position of 1,2-linked mannoses of glycans, followed by treatment with isolated N-acetylglucosamine-1-phosphodiester α-N-acetylglucosaminidase (phosphodiester α-GlcNAcase), which catalyses the removal of N-acetylglucosamine from the GlcNAc-phosphate modified glycan to generate a terminal mannose-6-phosphate on the glycan. WO 03/056897 experimentally demonstrates that GCase binding to mannose-6-phosphate receptor linked to a Sepharose® column is increased by the phosphorylation treatment. WO 03/056897 does not investigate the phosphorylated GCase in any biological system.

U.S. Pat. No. 8,926,967 and Dodge et al. (Intracerebroventricular infusion of acid sphingomyelinase corrects CNS manifestations in a mouse model of Niemann-Pick A disease. Experimental Neurology 2009, vol. 215, 349-357) concern intracerebroventricular administration of the lysosomal enzyme acid sphingomyelinase (ASM) in acid sphingomyelinase knock-out (ASMKO) mice. The preparation and structure of the ASM enzyme are not disclosed.

### SUMMARY

The present invention provides glucocerebrosidase preparations, uses thereof as well as methods employing such. The inventors experimentally confirmed that the present glucocerebrosidase preparations represent avenues for therapeutic interventions in conditions involving glucocerebrosidase deficiency, such as Gaucher disease and glucocerebrosidase-associated alpha-synucleinopathies.

Accordingly, in an aspect, the invention provides a glucocerebrosidase preparation or a composition comprising glucocerebrosidase, wherein at least 30% of glycans comprised by the glucocerebrosidase comprise at least one mannose-6-phosphate moiety.

A further aspect provides a glucocerebrosidase preparation or a composition comprising glucocerebrosidase, wherein at least 30% of glycans comprised by the glucocerebrosidase comprise at least one mannose-6-phosphate moiety, for use in therapy.

A related aspect provides a method for treating a subject in need thereof, the method comprising administering to the subject a prophylactically or therapeutically effective amount of a glucocerebrosidase preparation or a composition comprising glucocerebrosidase, wherein at least 30% of glycans comprised by the glucocerebrosidase comprise at least one mannose-6-phosphate moiety.

Another aspect provides a glucocerebrosidase preparation or a composition comprising glucocerebrosidase, wherein at least 10% of glycans comprised by the glucocerebrosidase comprise two mannose-6-phosphate moieties.

A further aspect provides a glucocerebrosidase preparation or a composition comprising glucocerebrosidase, wherein at least 10% of glycans comprised by the glucocerebrosidase comprise two mannose-6-phosphate moieties, for use in therapy.

A related aspect provides a method for treating a subject in need thereof, the method comprising administering to the subject a prophylactically or therapeutically effective amount of a glucocerebrosidase preparation or a composition comprising glucocerebrosidase, wherein at least 10% of glycans comprised by the glucocerebrosidase comprise two mannose-6-phosphate moieties.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 schematically illustrates human glucocerebrosidase (GCase) variants used in preclinical studies. “L2pre” denotes the signal peptide from the Yarrowia lipolytica (YL) lipase 2 (Lip2) protein; “GCase” denotes the human GCase portion of the polypeptide; “His8” or “H8” denote the poly-histidine tag 8×His; “H145L” and “K321N” denote amino acid substitutions compared to human wild-type (WT) GCase sequence.

FIG. 2 illustrates a representative DSA-FACE electropherogram of the isolated N-glycans of one of the uncapped and demannosylated GCase polypeptides embodying the invention, including peak annotation (M=mannose residue, P=phosphate residue).

FIG. 3 illustrates modular representation of the PMan₃GlcNAc₂ (Man 3-P), PMan₄GlcNAc₂ (Man 4-P), PMan₅GlcNAc₂ (Man 5-P), P₂Man₅GlcNAc₂ (Man5-(P)²), and P₂Man₆GlcNAc₂ (Man6-(P)²) N-glycan structures annotated in FIG. 2. Circles=mannose residues, squares=N-acetylglycosamine (GlcNAc) residue, wave=attachment point to the protein backbone.

FIG. 4 illustrates a representative DSA-FACE electropherogram of the isolated N-glycans of one of the uncapped and demannosylated GCase polypeptides embodying the invention (top panel), Cerezyme® (middle panel), and VPRIV® (bottom panel), including annotation of peaks corresponding to bi-phosphorylated (2P), monophosphorylated (1P) and non-phosphorylated (Neutral)N-glycans.

FIG. 5 illustrates comparison of net mannose-6-phosphate (M6P)-mediated uptake of OxyGCase, Cerezyme®, or VPRIV® by human neuroblastoma cells. Circles=OxyGCase, diamonds=Cerezyme®, triangles=VPRIV®.

FIG. 6 illustrates comparison of uptake of OxyGCase and Cerezyme® by mouse microglia, either with or without the addition of M6P or the combination of M6P and mannan.

FIG. 7 illustrates plasma pharmacokinetics (PK) curves of OxyGCase intracerebroventricularly (ICV) infused (either via bolus injection or slow infusion) into Gba1 D409V KI mice, as determined by 4MUβGlc activity assay.

FIG. 8 illustrates plasma pharmacokinetics (PK) curves of OxyGCase intracerebroventricularly (ICV) infused into Gba1 D409V KI mice, as determined by 4MUβGlc activity assay, comparing 1^{st} vs. 4^{th} ICV bolus (left panel), or 1^{st} vs. 4^{th} slow infusion (right panel).

FIG. 9 illustrates cyclophellitol-epoxide type activity-based probe (ABP), red MDW941 (left panel); and mechanism of irreversible inhibition of GCase by β-epoxide ring opening, A=nucleophile, B=general acid/base catalyst (right panel).

FIG. 10 illustrates distribution of unilateral ICV infused ABP-labelled GCaseMut1-H8 in the wild-type mouse brain, either infused for 2 minutes (2 m) or 20 minutes (20 m) at a flow rate of 1 μL/min resp. 0.1 μL/min.

FIG. 11 illustrates coronal brain slices (˜100 μm) at the level of the infusion site (scanned with FLA-5000 scanner after drying of sections) of the wild-type mouse brain unilaterally ICV-infused with ABP-labelled GCaseMut1-H8 for 2 minutes (2 min) or 20 minutes (20 min) at a flow rate of 1 U/min resp. 0.1 μL/min.

FIG. 12 illustrates relative distribution of ABP-labelled GCaseMut1-H8 to the CSF, brain and liver at 1 or 3 hours after ICV infusion for 2 minutes.

FIG. 13 illustrates GCase activity as determined by 4MUβGlc assay in brain parenchyma versus ventricular fraction, 3 hours after the last of repetitive every other day (EOD) unilateral ICV treatments with GCaseMut1-H8. WT=wild-type mice, KI=Gba1 D409V knock-in (KI) mice, n=number of animals studied per group.

FIG. 14 illustrates GCase activity as determined by 4MUβGlc assay in brain striatum versus cortex 48 h after the last of repetitive unilateral ICV treatments with GCaseMut1-H8 or Cerezyme®. WT=wild-type mice, KI=Gba1 D409V knock-in (KI) mice, n=number of animals studied per group, EW=weekly, BW=bi-weekly (i.e. two infusions per week).

FIG. 15 illustrates GCase activity in brain hemispheres (A) or liver (B) 48 h after the last of repetitive unilateral ICV treatments with the indicated OxyGCase variants and Cerezyme®. Results were obtained from different in vivo experiments. All treatments were performed via bolus injection of 10-20 min, except the group indicated with ‘in’ for which OxyGCase was infused slowly over a period of 3 hrs. WT=wild-type mice, KI=Gba1 D409V knock-in (KI) mice, EW=weekly, BW=bi-weekly (i.e. two infusions per week).

FIG. 16 illustrates human GCase protein levels as determined by alphaLISA in brain parenchyma versus ventricular fraction, 3 h after the last of repetitive every other day (EOD) unilateral ICV treatments with GCaseMut1-H8. WT=wild-type mice, KI=Gba1 D409V knock-in (KI) mice.

FIG. 17 illustrates overview of the in vivo efficacy upon unilateral ICV injection of OxyGCase variants and Cerezyme® as determined by HexSph levels in the brain. Results are expressed as percent HexSph of Gba1 D409V KI control levels. P-values versus WT and KI control are indicated in the bottom and top lines, respectively: *** p<0.001; ** p<0.01, * p<0.05, ns p>0.05 (one-way ANOVA & post hoc Bonferroni with correction for multiple comparisons). n=the total number of samples analyzed per study group and were pooled from different in vivo experiments. WT=wild-type mice, KI=Gba1 D409V knock-in (KI) mice, EW=weekly, BW=bi-weekly (i.e. two infusions per week), EOD=every other day, ABX=Ambroxol.

FIG. 18 illustrates correlation between HexSph and GCase activity levels in the brain of individual mice.

FIG. 19 illustrates GlcSph levels in pg per mg tissue (calculated by subtracting the vehicle-treated WT HexSph levels from the KI levels) upon repetitive GCaseMut1-H8 and Cerezyme® treatment in different brain regions. GlcSph as % of control KI levels is written above each data point. KI=Gba1 D409V knock-in (KI) mice, EW=weekly, BW=bi-weekly (i.e. two infusions per week).

FIG. 20 illustrates HexSph levels (in pg/mg cells, assuming a weight of 1 mg per 10⁶ cells (Sender et al. Revised Estimates for the Number of Human and Bacteria Cells in the Body. PLoS Biol. 2016, vol. 14, e1002533)) as determined by RP-LC-Q-TOF-MS analysis in samples after cell sorting of brain hemispheres. WT=wild-type mice, KI=Gba1 D409V knock-in (KI) mice, EW=weekly.

FIG. 21 illustrates overview of the in vivo efficacy upon unilateral ICV injection of OxyGCase variants and Cerezyme® as determined by HexSph levels in the liver. Results are expressed as percent HexSph of Gba1 D409V KI control levels. P-values versus WT and KI control are indicated in the bottom and top lines, respectively: *** p<0.001; ** p<0.01, * p<0.05, ns p>0.05 (one-way ANOVA and post-hoc Bonferroni with multiple comparison correction). n=total number of samples analyzed per study group and were pooled from different in vivo experiments. WT=wild-type mice, KI=Gba1 D409V knock-in (KI) mice, EW=weekly, EOD=every other day, ABX=Ambroxol.

FIG. 22 illustrates anti-GCase antibody titers in plasma from ICV OxyGCase-treated Gba1 D409V KI mice. The titer was an interpolation of the plate cut point (3 times the average of naive plasma from different mice at the lowest dilution used). Plasma samples were from 2 independent experiments and were collected at different time points: pre-dose and 24 h for all groups, and additionally after 6 h (8th bolus) or 48 h (12th bolus). n=number of mice per group.

FIG. 23 illustrates schematic representation of a Yarrowia-specific expression construct. ORF: open reading frame; ORI: origin of replication; Y1: Yarrowia lipolytica; zeta1/2: Yarrowia-specific sequences that increase the rate of random integration into the Yarrowia genome. Plasmids are digested with NotI to remove the bacterial sequences before transformation towards Yarrowia.

FIG. 24 shows GCase activity in the liver of mice, 24 h after the last of 4 weekly IV treatments with OxyGCase variants and Cerezyme®. The presented results are a combination of different in vivo experiments.

FIG. 25 shows HexSph levels (pg/mg tissue) in liver (top left), spleen (top right), heart (bottom left) and lung (bottom right) 24 h after the last of 4 weekly IV injections of vehicle or 30 U/kg huGCase(K321N), huGCase or Cerezyme® in WT or Gba1 D409V KI mice. P-values versus WT and KI control are indicated in the top and bottom lines, respectively: *** p<0.001; ** p<0.01, * p<0.05, ns p>0.05 (one-way ANOVA and post-hoc Bonferroni with multiple comparison correction). Data are obtained from 2 independent in vivo experiments.

FIG. 26 shows males and females combined active GCase concentration-time curves in CSF (left panel) and plasma (right panel) over 72 hours after 1, 4, 8, 12 and 19 ICV infusions with 10 mg or 50 mg Oxy5595 (huGCase(K321N) as described in the Examples) in non-human primates (NHPs). The dotted line indicates the limit of quantification of the assay (GCase activity measurement with the synthetic substrate, 4MUβGlc).

FIG. 27 provides overview of the brain punches. 1=Frontal cortex, 2=Striatum-nucleus caudatus, 3=Parietal cortex, 4=Thalamus, 5=Hippocampus, 6=Pons, 7=Medulla Oblongata, 8=Occipital cortex, 9 and 10=Cerebellum.

FIG. 28 shows active GCase levels (ng per g brain tissue), as determined with 4MUβGlc assay, in different brain regions of NHPs 48 h after the 23th ICV treatment with vehicle, 10 mg or 50 mg Oxy5595.

FIG. 29 shows concentration of active GCase in different brain regions of NHPs 48 h after the 23th ICV treatment with vehicle, 10 mg or 50 mg Oxy5595, as determined with 4MUβGlc assay.

FIG. 30 shows percent increase versus vehicle of active GCase in different brain regions of NHPs 48 h after the 23th ICV treatment with 10 mg or 50 mg Oxy5595.

FIG. 31 shows schematic representation of Oxy5595 distribution in the cynomolgus brain upon 23 ICV treatments with 50 mg Oxy5595. Sagittal midline section (left panel) and coronal section (right panel).

FIG. 32 shows the amount of active Oxy5595 that reaches different brain regions in mice compared to NHPs.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The experimental data included in the present specification demonstrate that several illustrative glucocerebrosidase preparations embodying the principles of the present invention resulted in fast and equal distribution of the GCase enzyme to both brain hemispheres, including deeper brain structures, where the GCase enzyme efficiently reduced the GCase substrate in all cell types including neurons, upon unilateral intracerebroventricular (ICV) treatment of a relevant animal (mouse) model of neuronopathic glucocerebrosidase deficiency, more particularly of type 3 Gaucher disease and Parkinson's disease. This was in sharp contrast to imiglucerase (Cerezyme®) which was not taken up by neuronal populations, and which therefore—similar to all other presently commercially available enzyme replacement therapies for Gaucher disease—does not represent a therapeutically viable avenue for treating the CNS-related symptoms of neuronopathic glucocerebrosidase deficiencies.

The inventors postulate that the latter is due to insufficient uptake of Cerezyme® by diseased neuronal cells. Cerezyme® and other currently available ERT therapies for Gaucher disease have comparatively low levels of monophosphorylated glycans, and virtually no detectable bi-phosphorylated glycans, but mainly neutral glycans. This may be adequate for cellular uptake by macrophages (Gaucher type 1) via the mannose receptor (MR), but as demonstrated herein is clearly unsatisfactory or ineffective for neuronal cells, which may only poorly express the MR on the plasma membrane. The inventors postulate that the comparatively higher degree of glycan phosphorylation in the GCase disclosed herein allows for efficient uptake by CNS cells including neurons via the mannose-6-phosphate (M6P) receptor.

Moreover, the illustrative GCase enzymes also reached peripheral organs in sufficient amounts to reduce substrate, corroborating that ICV treatment using the GCase compositions taught herein can improve the peripheral symptoms of the disease as well, advantageously avoiding the need for a combined ICV and systemic (intravenous) treatment approach.

Accordingly, provided herein is a glucocerebrosidase (GCase) preparation, wherein at least 30% of glycans comprised by the glucocerebrosidase comprise at least one mannose-6-phosphate moiety. Also provided herein is a composition comprising glucocerebrosidase, wherein at least 30% of glycans comprised by the glucocerebrosidase comprise at least one mannose-6-phosphate moiety. In certain embodiments, in ascending order of preference, at least 40%, or at least 50%, or at least 60%, or at least 70%, or at least 80%, or at least 90%, or at least 95%, or at least 98%, or at least 99%, or substantially all of the glycans comprised by said glucocerebrosidase comprise at least one mannose-6-phosphate moiety. In certain embodiments, at least some of the mannose-6-phosphate moiety-comprising glycans comprise two mannose-6-phosphate moieties. In certain embodiments, in ascending order of preference, at least 5%, or at least 10%, or at least 15%, or at least 20%, or at least 25%, or at least 30%, or at least 35%, or at least 40%, or at least 45% of the mannose-6-phosphate moiety-comprising glycans comprise two mannose-6-phosphate moieties. Hence, in certain embodiments, at least 30% of glycans comprised by the glucocerebrosidase comprise at least one mannose-6-phosphate moiety and, in ascending order of preference, at least 5%, or at least 10%, or at least 15%, or at least 20%, or at least 25%, or at least 30%, or at least 35%, or at least 40%, or at least 45% of the mannose-6-phosphate moiety-comprising glycans comprise two mannose-6-phosphate moieties. In certain embodiments, in ascending order of preference, at least 40%, or at least 50%, or at least 60%, or at least 70%, or at least 80%, or at least 90%, or at least 95%, or at least 98%, or at least 99%, or substantially all of the glycans comprised by said glucocerebrosidase comprise at least one mannose-6-phosphate moiety and, in ascending order of preference, at least 5%, or at least 10%, or at least 15%, or at least 20%, or at least 25%, or at least 30%, or at least 35%, or at least 40%, or at least 45% of the mannose-6-phosphate moiety-comprising glycans comprise two mannose-6-phosphate moieties. In certain embodiments, at least 40% of the glucocerebrosidase molecules are glycosylated. In certain embodiments, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated. Hence, in certain embodiments, at least 30% of glycans comprised by the glucocerebrosidase comprise at least one mannose-6-phosphate moiety and at least 40% of the glucocerebrosidase molecules are glycosylated. In certain embodiments, at least 30% of glycans comprised by the glucocerebrosidase comprise at least one mannose-6-phosphate moiety and, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated. In certain embodiments, in ascending order of preference, at least 40%, or at least 50%, or at least 60%, or at least 70%, or at least 80%, or at least 90%, or at least 95%, or at least 98%, or at least 99%, or substantially all of the glycans comprised by said glucocerebrosidase comprise at least one mannose-6-phosphate moiety and, in ascending order of preference, at least 40% of the glucocerebrosidase molecules are glycosylated. In certain embodiments, in ascending order of preference, at least 40%, or at least 50%, or at least 60%, or at least 70%, or at least 80%, or at least 90%, or at least 95%, or at least 98%, or at least 99%, or substantially all of the glycans comprised by said glucocerebrosidase comprise at least one mannose-6-phosphate moiety and, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated.

Further provided herein is a glucocerebrosidase preparation, wherein at least 10% of glycans comprised by the glucocerebrosidase comprise two mannose-6-phosphate moieties. Also provided herein is a composition comprising glucocerebrosidase, wherein at least 10% of glycans comprised by the glucocerebrosidase comprise two mannose-6-phosphate moieties. In certain embodiments, more than 10% of glycans comprised by the glucocerebrosidase comprise at least one mannose-6-phosphate moiety. In certain embodiments, in ascending order of preference, at least 15%, or at least 20%, or at least 25%, or at least 30%, or at least 35%, or at least 40%, or at least 45% of the glycans comprised by the glucocerebrosidase comprise two mannose-6-phosphate moieties. In certain embodiments, in ascending order of preference, at least 15%, or at least 20%, or at least 25%, or at least 30%, or at least 35%, or at least 40%, or at least 45% of the glycans comprised by the glucocerebrosidase comprise two mannose-6-phosphate moieties, and, respectively, more than 15%, or more than 20%, or more than 25%, or more than 30%, or more than 35%, or more than 40%, or more than 45% of glycans comprised by the glucocerebrosidase comprise at least one mannose-6-phosphate moiety. In certain embodiments, in ascending order of preference, at least 20%, or at least 30%, or at least 40%, or at least 50%, or at least 60%, or at least 70%, or at least 80%, or at least 90%, or at least 95%, or at least 98%, or at least 99%, or substantially all of the glycans comprised by the glucocerebrosidase comprise at least one mannose-6-phosphate moiety. Hence, in certain embodiments, at least 10% of glycans comprised by the glucocerebrosidase comprise two mannose-6-phosphate moieties, and, in ascending order of preference, at least 20%, or at least 30%, or at least 40%, or at least 50%, or at least 60%, or at least 70%, or at least 80%, or at least 90%, or at least 95%, or at least 98%, or at least 99%, or substantially all of the glycans comprised by the glucocerebrosidase comprise at least one mannose-6-phosphate moiety. In certain embodiments, at least 40% of the glucocerebrosidase molecules are glycosylated. In certain embodiments, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated. Hence, in certain embodiments, at least 10% of glycans comprised by the glucocerebrosidase comprise two mannose-6-phosphate moieties, and least 40% of the glucocerebrosidase molecules are glycosylated. In certain embodiments, at least 10% of glycans comprised by the glucocerebrosidase comprise two mannose-6-phosphate moieties and, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated. In certain embodiments, in ascending order of preference, at least 15%, or at least 20%, or at least 25%, or at least 30%, or at least 35%, or at least 40%, or at least 45% of the glycans comprised by the glucocerebrosidase comprise two mannose-6-phosphate moieties, and at least 40% of the glucocerebrosidase molecules are glycosylated. In certain embodiments, in ascending order of preference, at least 15%, or at least 20%, or at least 25%, or at least 30%, or at least 35%, or at least 40%, or at least 45% of the glycans comprised by the glucocerebrosidase comprise two mannose-6-phosphate moieties and, in ascending order of preference, at least 50%, or at least 60%, or at least 70%, or at least 80%, or at least 90%, or at least 95%, or at least 98%, or at least 99%, or substantially all of the glucocerebrosidase molecules are glycosylated.

Where percentages of certain generic or specific glycan structures comprised by the GCase are recited, such as the percentage of glycans that comprise at least one mannose-6-phosphate moiety, or the percentage of glycans that comprise two mannose-6-phosphate moieties, a percentage by number (or molar amount) may be particularly meant. By means of an example, if 50 or more glycans in a plurality of 100 glycans comprise a mannose-6-phosphate moiety, the plurality can be said to comprise at least 50% glycans comprising at least one mannose-6-phosphate moiety. Hence, the percentages are based on the group or pool of glycans contained by the plurality of glucocerebrosidase molecules comprised by the preparation or composition. Such percentages may be readily determined from a representative sample of the glucocerebrosidase preparation or composition using methods illustrated in the Examples, such as by releasing glycans from the GCase with N-Glycosidase F (PNGaseF) treatment, labelling the glycans with APTS (8-amino-1,3,6-pyrenetrisulfonic acid trisodium salt), and determining the glycan structures using DSA-FACE (DNA Sequencer-Aided Fluorophore-Assisted Carbohydrate Electrophoresis). DSA-FACE separates the glycans by charge and mass, and provides a peak profile read-out, where each peak represents a given glycan structure. The peak area gives a relative representation of the amount of each N-glycan structure. Typically, the percentage of a given glycan structure by number or molar amount may approximate its percentage by weight, and in any event the skilled person can calculate and convert between both types of percentages based on the molecular weight of the respective glycan structures.

Where percentages of glycosylated GCase molecules are recited, a percentage by number (or molar amount) may be particularly meant. By means of an example, if 50 or more GCase molecules in a plurality of 100 GCase molecules are glycosylated, the plurality can be said to comprise at least 50% glycosylated GCase molecules. Glycosylated vs. non-glycosylated GCase molecules may be suitably separated and quantified for example based on their different electrophoretic mobility. Typically, the percentage of glycosylated GCase may approximate its percentage by weight, and in any event the skilled person can calculate and convert between both types of percentages based on the molecular weight of the respective GCase molecules.

The invention is thus embodied by glucocerebrosidase proteins or polypeptides as disclosed herein. The terms “peptide”, “polypeptide”, or “protein” can be used interchangeably and relate to any natural, synthetic, or recombinant molecule comprising amino acids joined together by peptide bonds between adjacent amino acid residues. A “peptide bond”, “peptide link” or “amide bond” is a covalent bond formed between two amino acids when the carboxyl group of one amino acid reacts with the amino group of the other amino acid, thereby releasing a molecule of water. The polypeptide can be from any source, e.g., a naturally occurring polypeptide, a chemically synthesized polypeptide, a polypeptide produced by recombinant molecular genetic techniques, or a polypeptide from a cell or translation system. Preferably, the polypeptide is a polypeptide produced by recombinant molecular genetic techniques. The polypeptide may be a linear chain or may be folded into a globular form. The terms “amino acid” and “amino acid residue” may be used interchangeably herein. Further, unless otherwise apparent from the context, reference herein to any peptide, polypeptide or protein may generally also encompass altered forms of said peptide, polypeptide or protein such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “glycan” broadly encompasses any mono-, oligo- or poly-saccharide in free form or forming a carbohydrate portion of a glycoconjugate molecule, such as a glycoprotein, proteoglycan or glycolipid. Monosaccharide units typically comprised in glycans, such as in glycoprotein glycans, may include mannose (Man), N-acetylglucosamine (GlcNAc), galactose (Gal), sialic acid (SA), xylose (Xyl), and/or fucose. Monosaccharide units typically found in fungal including yeast cell glycans may include Man and GlcNAc. Linkages between monosaccharides in glycans may be in α- and/or β-form, chains may be linear or branched, and optional glycan modifications may typically include acetylation, phosphorylation, and/or sulphation. Glycoproteins carry one or more glycans covalently attached to the polypeptide via N- or O-linkage. In certain preferred embodiments, glycans as intended herein may be N-glycans. A protein or polypeptide which comprises at least one glycan, more particularly at least one glycan covalently linked thereto, even more particularly at least one N- or O-linked glycan, is commonly referred to as “glycosylated”. By means of an example, a GCase molecule which comprises at least one O- or N-linked glycan, preferably at least one N-linked glycan, such as in certain embodiments at least one N-linked glycan and no O-linked glycans, may be denoted as “glycosylated” GCase molecule. O-glycans are linked to hydroxyl groups of serine or threonine residues. N-glycans are linked via a side-chain nitrogen to an asparagine residue. Naturally-occurring N-glycans share a common penta-saccharide region of two mannose residues, linked separately by α-1,3 and α-1,6 linkages to a central mannose, which in turn is linked by a β-1,4 linkage to a chitobiose core consisting of two β-1,4-linked GlcNAc residues. Based on further processing of the penta-saccharide, N-glycans are divided into three main classes: (i) high-mannose, (ii) complex, and (iii) hybrid types.

In certain embodiments, a glycosylated GCase molecule may carry at least one, such as exactly one, glycan, more particularly N-glycan; or preferably may carry at least two, such as exactly two, glycans, more particularly N-glycans; or may more preferably carry at least three, such as exactly three, glycans, more particularly N-glycans; or may even more preferably carry at least four, such as exactly four, glycans, more particularly N-glycans. For example, wild-type human GCase contains four N-glycosylation sites, but may be engineered to include additional N-glycosylation sites, such as taught in WO 01/49830. In certain embodiments, a glycosylated GCase molecule may carry more than four, such as exactly five, six, seven, eight, nine, or ten glycans, more particularly N-glycans. A plurality of glycosylated GCase molecules may include GCase molecules each independently carrying one or more glycans, more particularly N-glycans. For example, a plurality of glycosylated GCase molecules may on average carry between 1.0 and 1.9, or between 2.0 and 2.9, or between 3.0 and 3.9, or about 4.0 glycans, more particularly N-glycans, per GCase molecule.

The phrase “comprises at least one mannose-6-phosphate moiety” denotes that a glycan, more particularly N-glycan, comprises one or more than one mannose-6-phosphate (M6P) moieties, such as exactly one or exactly two M6P moieties. The phrase “comprises two mannose-6-phosphate moieties” denotes that a glycan, more particularly N-glycan, comprises two, such as exactly two, M6P moieties. Such M6P moiety is linked to an underlying monosaccharide unit of the glycan, such as to an underlying mannose unit of the glycan, by a covalent bond, such as a glycosidic bond, more typically α-1,2 or α-1,6 glycosidic bond. In the M6P moiety, the phosphate group is linked to C6 of the mannose group. The phosphate group is exposed (e.g., is not “capped” by another monosaccharide unit, such as by another mannose unit). For illustration, a representative structure of mannose-6-phosphate is shown below:

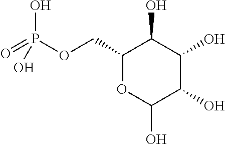 [SMILES: O=P(O)(O)OC[C@H]1OC(O)[C@@H](O)[C@@H](O)[C@@H]1O]

The phosphate group may be in a free acid form (—OPO(OH)₂, or dissociated to —OPO₂(OH)⁻ and H+, or to —OPO₃²⁻ and 2H+), or may be in the form of salts, in particular pharmaceutically acceptable salts, e.g., may be converted into metal or amine addition salt forms by treatment with appropriate organic and inorganic bases.

In certain embodiments, a glycan, more particularly N-glycan, comprising at least one, such as exactly one, mannose-6-phosphate moiety, comprises or consists of at least a core structure selected from:

P-6Manα1-6Manα1-6Manβ1-4GlcNAcβ1-4GlcNAc (formula I); or

P-6Manα1-2Manα1-3Manβ1-4GlcNAcβ1-4GlcNAc (formula II);

wherein α1-2, α1-3, α1-6, and β1-4 denote glycosidic bonds between the neighbouring monosaccharide units. These structures are also modularly illustrated in FIG. 3, panel ‘Man 3-P’, where formula I corresponds to the right-hand structure, and formula II to the left-hand structure.

In certain embodiments, a glycan, more particularly N-glycan, comprising two, such as exactly two, mannose-6-phosphate moieties, comprises or consists of at least a core structure:

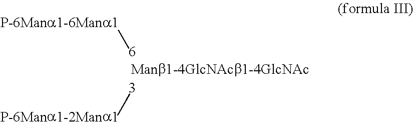

wherein α1-2, α1-3, α1-6, and β1-4 denote glycosidic bonds between the neighbouring monosaccharide units. This structure is also modularly illustrated in FIG. 3, panel ‘Man 5-(P)²’.

In certain preferred embodiments, the mannose of the mannose-6-phosphate moiety is a terminal mannose. The mannose will thus form a glycosidic bond with an underlying monosaccharide unit in the glycan, but will not be interposed between the underlying monosaccharide unit and another, ensuing monosaccharide unit. Typically, the glycosidic bond may be an α-glycosidic bond, more particularly an α-glycosidic bond via the mannose's C1 atom. Typically, the underlying monosaccharide unit may be mannose. Typically, the glycosidic bond may be a α-1,2 or α-1,6 glycosidic bond to an underlying mannose.

In certain preferred embodiments, the mannose-6-phosphate moiety-comprising glycans are each independently selected from the group comprising or consisting of PMan₇GlcNAc₂, PMan₆GlcNAc₂, PMan₅GlcNAc₂, PMan₄GlcNAc₂, PMan₃GlcNAc₂, P₂Man₆GlcNAc₂, and P₂Man₅GlcNAc₂. For example, at least 50%, at least 60%, at least 70%, at least 80%, at least 90%, at least 95%, at least 98%, at least 99%, or substantially all mannose-6-phosphate moiety-comprising glycans may be each independently selected from these structures. The structure of such N-glycans may be obtained by notionally hydrolysing one or (where applicable sequentially) more terminal mannose residues other than the Man-6-P residue from the structures PMan₈GlcNAc₂ or P₂Man₈GlcNAc₂, shown below:

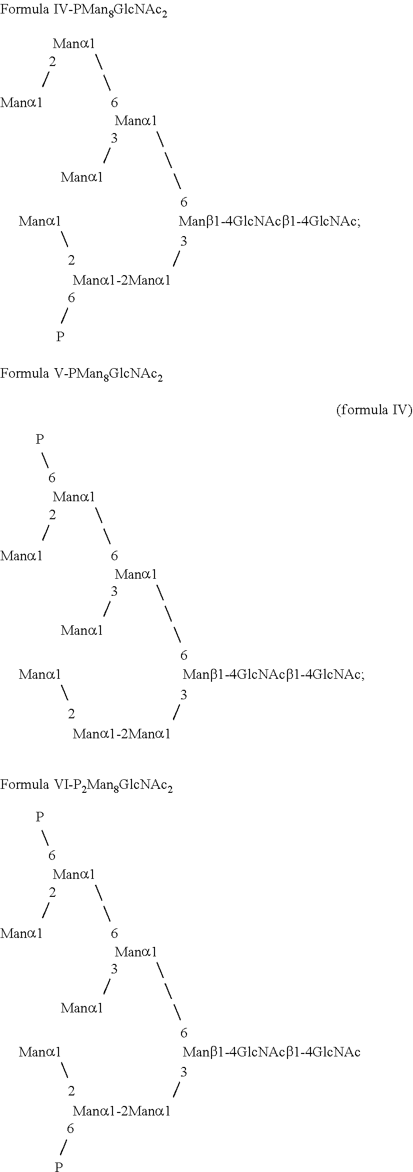

In certain preferred embodiments, the mannose-6-phosphate moiety-comprising glycans are each independently selected from the group comprising or consisting of PMan₅GlcNAc₂, PMan₄GlcNAc₂, PMan₃GlcNAc₂, P₂Man₆GlcNAc₂, and P₂Man₅GlcNAc₂, also modularly shown in FIG. 3. For example, at least 50%, at least 60%, at least 70%, at least 80%, at least 90%, at least 95%, at least 98%, at least 99%, or substantially all mannose-6-phosphate moiety-comprising glycans may be each independently selected from these structures.

In certain preferred embodiments, the mannose-6-phosphate moiety-comprising glycans are each independently selected from the group comprising or consisting of PMan₃GlcNAc₂ and P₂Man₅GlcNAc₂, as modularly shown in FIG. 3, and also shown in formulas I and II, and III, respectively. For example, at least 50%, at least 60%, at least 70%, at least 80%, at least 90%, at least 95%, at least 98%, at least 99%, or substantially all mannose-6-phosphate moiety-comprising glycans may be each independently selected from these structures.

The terms “glucocerebrosidase”, “β-glucocerebrosidase”, “GCase”, “GC”, or “glucosylceramidase” broadly encompass enzymes (EC 3.2.1.45) which catalyse hydrolysis of the glucosidic linkage in glucose-containing glycolipids, such as glucosylceramide and glucosylsphingosine.

In certain preferred embodiments, the glucocerebrosidase is human wild-type glucocerebrosidase. The qualifier “human” as used herein in connection with the GCase polypeptide relates to the primary amino acid sequence of the GCase polypeptide, rather than to its origin or source. For example, the human GCase polypeptide may be obtained by technical means, e.g., by recombinant expression, cell-free translation, or non-biological peptide synthesis. As used herein, the term “wild-type” as applied to a nucleic acid or polypeptide refers to a nucleic acid or a polypeptide that occurs in, or is produced by, a biological organism as that biological organism exists in nature. The term “wild-type” may be synonymous with “native”, the latter encompassing nucleic acids or polypeptides having a native sequence, i.e., ones of which the primary sequence is the same as that of the nucleic acids or polypeptides found in or derived from nature. A skilled person understands that native sequences may differ between or within different individuals of the same species due to normal genetic diversity (variation) within a given species. Also, native sequences may differ between or within different individuals of the same species due to post-transcriptional or post-translational modifications. Any such variants or isoforms of nucleic acids or polypeptides are encompassed herein as being “native”. Accordingly, all sequences of nucleic acids or polypeptides found in or derived from nature are considered “native”. The term “native” encompasses the nucleic acids or polypeptides when forming a part of a living organism, organ, tissue or cell, when forming a part of a biological sample, as well as when at least partly isolated from such sources. The term also encompasses the nucleic acids or polypeptides when produced by recombinant or synthetic means. However, even though most native human GCase nucleic acids or polypeptides may be considered “wild-type”, those carrying naturally-occurring mutations associated with or causing a disease phenotype, such as Gaucher disease or α-synucleinopathies such as Parkinson's disease (such mutations may diminish or eliminate the expression and/or activity of GCase), are generally excluded from the scope of the term “wild-type”. Hence, in certain embodiments, human GCase is not one associated with or causing a disease phenotype.

Human glucocerebrosidase is a soluble lysosomal enzyme which has been described in the literature, such as in Lieberman (Enzyme Res. 2011, article ID 973231). Gene names for human GCase include “GBA”, “GC”, and “GLUC”. Exemplary human GCase protein sequence may be as annotated under U.S. government's National Center for Biotechnology Information (NCBI) Genbank (http://www.ncbi.nlm.nih.gov/) accession number NP_000148.2 (sequence version 2), or Swissprot/Uniprot (http://www.uniprot.org/) accession number P04062-1. Exemplary human GCase mRNA (cDNA) sequence may be as annotated under NCBI Genbank accession number NM_000157.4 (sequence version 4).

The human GCase amino acid sequence annotated under NP_000148.2 is reproduced below:

(SEQ ID NO: 1)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQ.

The above representative human GCase polypeptide sequence is that of a GCase precursor, including an N-terminal signal peptide. During processing of human GCase, the signal peptide, corresponding to amino acids 1 to 39 in SEQ ID NO: 1, is processed away to form the mature human GCase protein, corresponding to amino acids 40 to 536 of SEQ ID NO: 1, which is thus 497-amino acids long. Hence, the amino acid sequence of an exemplary mature human GCase is reproduced below:

(SEQ ID NO: 2)
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSPPA
QNLLLKSYFSEEGIGYNIIRVPMASCDFSIRTYTYADTPDDFQLHNFSLP
EEDTKLKIPLIHRALQLAQRPVSLLASPWTSPTWLKTNGAVNGKGSLKGQ
PGDIYHQTWARYFVKFLDAYAEHKLQFWAVTAENEPSAGLLSGYPFQCLG
FTPEHQRDFIARDLGPTLANSTHHNVRLLMLDDQRLLLPHWAKVVLTDPE
AAKYVHGIAVHWYLDFLAPAKATLGETHRLFPNTMLFASEACVGSKFWEQ
SVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALM
HPDGSAVVVVLNRSSKDVPLTIKDPAVGFLETISPGYSIHTYLWRRQ.

Reference to human GCase polypeptide as used herein encompasses both human GCase precursor polypeptides and mature human GCase polypeptides, as apparent from the context. Furthermore, human GCase polypeptides in which the native signal peptide is replaced by a signal peptide active in a suitable host cell (e.g., signal peptide active in fungal cells), are also encompassed, as apparent from the context. In certain embodiments, the human wild-type glucocerebrosidase comprises or consists of the amino acid sequence as set forth in SEQ ID NO: 2.

In certain embodiments, the glucocerebrosidase is a biologically active variant or fragment of human wild-type glucocerebrosidase. The expressions “biologically active variants or fragments” or “functionally active variants or fragments” of human wild-type GCase polypeptide comprises functionally active variants of the human wild-type GCase polypeptide, functionally active fragments of the human wild-type GCase polypeptide, as well as functionally active variants of fragments of the human wild-type GCase polypeptide.

The term “fragment” of a protein, polypeptide, or peptide generally refers to N-terminally and/or C-terminally deleted or truncated forms of said protein, polypeptide or peptide. The term encompasses fragments arising by any mechanism, such as, without limitation, by alternative translation, exo- and/or endo-proteolysis and/or degradation of said peptide, polypeptide or protein, such as, for example, in vivo or in vitro, such as, for example, by physical, chemical and/or enzymatic proteolysis. Without limitation, a fragment of a protein, polypeptide, or peptide may represent at least about 5% (by amino acid number), or at least about 10%, e.g., 20% or more, 30% or more, or 40% or more, such as preferably 50% or more, e.g., 60% or more, 70% or more, 80% or more, 90% or more, or 95% or more of the amino acid sequence of said protein, polypeptide, or peptide, e.g., a corresponding human wild-type GCase polypeptide, e.g., a corresponding mature human wild-type GCase polypeptide, e.g., human wild-type GCase polypeptide as set forth in SEQ ID NO: 2.

For example, a fragment of a protein, polypeptide, or peptide may include a sequence of 5 or more consecutive amino acids, 10 or more consecutive amino acids, 20 or more consecutive amino acids, 30 or more consecutive amino acids, e.g., 40 or more consecutive amino acids, such as for example 50 or more consecutive amino acids, 60 or more, 70 or more, 80 or more, 90 or more, 100 or more, 200 or more, 300 or more, 310 or more, 320 or more, 330 or more, 340 or more, 350 or more, 360 or more, 370 or more, 380 or more, 390 or more, 400 or more, 410 or more, 420 or more, 430 or more, 440 or more, 450 or more, 460 or more, 470 or more, 480 or more, or 490 or more consecutive amino acids of the corresponding full-length protein or polypeptide, e.g., a corresponding human wild-type GCase polypeptide, e.g., a corresponding mature human wild-type GCase polypeptide, e.g., human wild-type GCase polypeptide as set forth in SEQ ID NO: 2.

In an embodiment, a fragment of a protein, polypeptide, or peptide may be N-terminally and/or C-terminally truncated by between 1 and about 20 amino acids, such as by between 1 and about 15 amino acids, or by between 1 and about 10 amino acids, or by between 1 and about 5 amino acids, compared with the corresponding full-length protein or polypeptide, e.g., a corresponding human wild-type GCase polypeptide, e.g., a corresponding mature human wild-type GCase polypeptide, e.g., human wild-type GCase polypeptide as set forth in SEQ ID NO: 2.

The term “variant” of a protein, polypeptide or peptide generally refers to proteins, polypeptides or peptides the amino acid sequence of which is substantially identical (i.e., largely but not wholly identical) to the sequence of the protein, polypeptide, or peptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical to the sequence of the protein, polypeptide, or peptide, e.g., to the sequence of a corresponding human wild-type GCase polypeptide, e.g., a corresponding mature human wild-type GCase polypeptide, e.g., human wild-type GCase polypeptide as set forth in SEQ ID NO: 2. Preferably, a variant may display such degrees of identity to a recited protein, polypeptide or peptide when the whole sequence of the recited protein, polypeptide or peptide is queried in the sequence alignment (i.e., overall sequence identity). Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and the amino acid sequence of a query polypeptide (e.g., human wild-type GCase polypeptide, e.g., mature human wild-type GCase polypeptide, e.g., human wild-type GCase polypeptide as set forth in SEQ ID NO: 2) will entail aligning the two amino acid sequences using the Blast 2 sequences (Bl2seq) algorithm, available as a web application or as a standalone executable programme (BLAST version 2.2.31+) at the NCBI web site (www.ncbi.nlm.nih.gov), using suitable algorithm parameters. An example of suitable algorithm parameters include: matrix=Blosum62, cost to open a gap=11, cost to extend a gap=1, expectation value=10.0, word size=3). If the two compared sequences share homology, then the output will present those regions of homology as aligned sequences. If the two compared sequences do not share homology, then the output will not present aligned sequences. Once aligned, the number of matches will be determined by counting the number of positions where an identical amino acid residue is presented in both sequences. The percent identity is determined by dividing the number of matches by the length of the query polypeptide, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by Bl2seq already conveniently includes the percentage of identities.

A variant of a protein, polypeptide, or peptide may be a homologue (e.g., orthologue or paralogue) of said protein, polypeptide, or peptide. As used herein, the term “homology” generally denotes structural similarity between two macromolecules, particularly between two proteins or polypeptides, from same or different taxons, wherein said similarity is due to shared ancestry.

A variant of a protein, polypeptide, or peptide may comprise one or more amino acid additions, deletions, or substitutions relative to (i.e., compared with) the corresponding protein or polypeptide, e.g., a corresponding human wild-type GCase polypeptide, e.g., a corresponding mature human wild-type GCase polypeptide, e.g., human wild-type GCase polypeptide as set forth in SEQ ID NO: 2.

For example, a variant (substitution variant) of a protein, polypeptide, or peptide may comprise up to 70 (e.g., not more than one, two, three, four, five, six, seven, eight, nine, ten, 12, 15, 20, 25, 30, 35, 40, 50, 60, or 70) conservative amino acid substitutions relative to (i.e., compared with) the corresponding protein or polypeptide, e.g., a corresponding human wild-type GCase polypeptide, e.g., a corresponding mature human wild-type GCase polypeptide, e.g., human wild-type GCase polypeptide as set forth in SEQ ID NO: 2.

A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

Alternatively or in addition, for example, a variant (deletion variant) of a protein, polypeptide, or peptide may lack up to 20 amino acid segments (e.g., one, two, three, four, five, six, seven, eight, nine, ten, 11, 12, 13, 14, 15, 16, 17, 18, 19, or 20 segments) relative to (i.e., compared with) the corresponding protein or polypeptide, e.g., a corresponding human wild-type GCase polypeptide, e.g., a corresponding mature human wild-type GCase polypeptide, e.g., human wild-type GCase polypeptide as set forth in SEQ ID NO: 2. The deletion segment(s) may each independently consist of one amino acid, two contiguous amino acids or three contiguous amino acids. The deletion segments may be non-contiguous, or two or more or all of the deletion segments may be contiguous.

A variant of a protein, polypeptide, or peptide may be a fusion protein, polypeptide, or peptide, wherein the protein, polypeptide, or peptide is chemically conjugated, non-covalently bound, or translationally fused to one or more other proteins, polypeptides or peptides. Other proteins, polypeptides or peptides may include signal-generating compounds (e.g. enzyme or fluorophore), diagnostic or detectable markers (e.g. green fluorescent protein (GFP), or chloramphenicol acetyl transferase (CAT)), amino acid sequences used for purification of recombinant proteins, polypeptides or peptides (e.g. FLAG, polyhistidine (e.g., hexahistidine), hemagluttanin (HA), glutathione-S-transferase (GST), or maltose-binding protein (MBP)), signal sequences and amino acid sequences used to direct or enhance the transport of the protein, polypeptide or peptide to a target cell (e.g. blood-brain barrier shuttle peptides), but are not limited thereto. The amino acid sequence can be fused at the N-terminus and/or C-terminus of the agonist as intended herein, optionally by use of a spacer (e.g. aminohexanoic acid (Ahx) or poly(ethylene)glycol (PEG)).

Where the present specification refers to or encompasses variants and/or fragments of proteins, polypeptides or peptides, this denotes variants or fragments which are functionally active or functional, i.e., which at least partly retain the biological activity or intended functionality of the respective or corresponding proteins, polypeptides, or peptides. By means of an example and not limitation, a functionally active variant or fragment of human wild-type GCase polypeptide as disclosed herein shall at least partly retain the biological activity of human wild-type GCase polypeptide. For example, it may retain one or more aspects of the biological activity of human wild-type GCase polypeptide, such as hydrolase activity. Preferably, a functionally active variant or fragment may retain at least about 20%, e.g., at least about 25%, or at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared with the corresponding protein, polypeptide, or peptide. Reference to the “activity” of a protein, polypeptide, or peptide such as human wild-type GCase polypeptide may generally encompass any one or more aspects of the biological activity of the protein, polypeptide, or peptide, such as without limitation any one or more aspects of its biochemical activity, enzymatic activity, signalling activity, interaction activity, ligand activity, and/or structural activity, e.g., within a cell, tissue, organ or an organism. By means of an example and not limitation, reference to the activity of human wild-type GCase polypeptide or functionally active variant or fragment thereof may particularly denote its activity as a hydrolase. Where the activity of a given protein, polypeptide, or peptide such as human wild-type GCase polypeptide can be readily measured in an established assay, e.g., an enzymatic assay (such as, for example, by a fluorimetric assay), a functionally active variant or fragment of the protein, polypeptide, or peptide may display activity in such assays, which is at least about 20%, e.g., at least about 25%, or at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the activity of the respective or corresponding protein, polypeptide, or peptide.

For example, the hydrolase activity of human wild-type GCase or functionally active variant or fragment thereof can be measured in an enzymatic assay, such as particularly 4MUβGlc (4-methyllumbelliferyl-β-D-glucopyranoside (Urban et al., 2008, Comb Chem High Throughput Screen, vol. 11(10), 817-824)) assay, a fluorescent assay that measures the enzymatic activity of GCase using the synthetic substrate 4MUβGlc. One unit is defined as the amount of enzyme that catalyses the hydrolysis of 1 μmol 4MUβGlc per minute, at 37° C. at a final substrate concentration of 5 mM in 111 mM Na₂HPO₄, 44 mM citric acid, 0.5% (w/v) BSA, 10 mM sodium taurocholate, 0.25% (v/v) Triton X-100, pH 5.5.

In certain examples, a functionally active variant or fragment of human wild-type GCase may have at least 25% (e.g., at least 30%, at least 40%, at least 50%, at least 60%, at least 70%, at least 75%, at least 80%, at least 85%, at least 90%, at least 95%, at least 98%, at least 99%, at least 100%, or even greater than 100%) of the GCase enzymatic activity of the human wild-type GCase polypeptide as set forth in SEQ ID NO: 2. The functional variant or fragment can generally, but not always, be comprised of a continuous region of the protein, wherein the region has functional activity.

The amino acid sequence of the active site of human GCase polypeptide has been described in the literature (Lieberman 2011). The residues forming the active site more particularly, residues involved in substrate recognition and binding (residues that line the glucose binding) are located in domain 2 and include Arg120, Asp127, Phe128, Trp179, Asn234, Tyr244, Phe246, Tyr313, Cys342, Ser345, Trp381, Asn396, Phe397, and Val398 (with amino acid numbering as in the mature protein). Candidate functional variants or fragments of human wild-type GCase polypeptides can therefore be produced by one skilled in the art using well established methods, such as homology modelling and computational engineering, and tested for the desired enzymatic activity.

Hence, in certain embodiments, the biologically active variant of human wild-type glucocerebrosidase displays at least 90% sequence identity to human wild-type glucocerebrosidase, such as at least 95% or at least 98% or at least 99% sequence identity, in particular overall sequence identity, to human wild-type glucocerebrosidase, such as that of SEQ ID NO: 2.

In certain embodiments, the biologically active variant of human wild-type glucocerebrosidase has increased stability and/or specificity relative to human wild-type glucocerebrosidase. In certain embodiments, the stability of the GCase variant may be increased by at least 1% compared with the stability of human wild-type GCase. For example, the stability may be increased by at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% or more (e.g., by at least 100%, at least 200% or at least 300%) compared with the stability of human wild-type GCase.

The stability of the GCase variant may be determined by a method comprising incubating the GCase variant for a certain time period (e.g., for 1 hour, 2 hours, 4 hours, 8 hours or 16 hours) at a certain temperature (e.g., at 37° C.), under certain conditions (e.g., in about neutral pH, e.g., pH 7.5, or in serum or plasma), and measuring the GCase activity. As a control, human wild-type GCase can be used. The enzyme activity at time zero can be set to be 100% under each condition. The stability of each enzyme can be calculated and expressed as the ratio (e.g., percent) of the enzyme activity at a particular incubation time point to the value at time zero.

Alternatively or in addition, the stability of the GCase variant may be predicted by a thermal shift assay, also called differential scanning fluorimetry (DSF).

Alternatively or in addition, the stability of the GCase variant may be predicted by measuring its melting temperature (Tm) of the protein or polypeptide. The “melting temperature (Tm)” of a protein or polypeptide refers to the temperature at which 50% of the protein or polypeptide is inactivated during reversible heat denaturation. By means of example, the melting temperature of a protein or polypeptide can be determined using fluorescence-based thermal shift assays (TSA). Such assays can be based on the use of SYPRO Orange, a dye that binds non-specifically to hydrophobic surfaces and whose fluorescence is quenched in an aqueous environment. During thermal induced unfolding, the fluorophore will preferentially bind to the exposed hydrophobic interior of an unfolding protein leading to a sharp decrease in quenching. Thermally induced unfolding is an irreversible process following a two-state model with a sharp transition between the folded and non-folded states, where Tm is defined as the midpoint of temperature of the protein-unfolding transition. By means of another example, the melting temperature of a protein or polypeptide can be determined using circular dichroism (CD) spectroscopy. The term “circular dichroism spectroscopy” generally refers to a tool to study the secondary structure of proteins or protein folding. Circular dichroism spectroscopy measures the absorption of circularly polarized light. In proteins, secondary structures such as alpha helices and beta sheets are chiral, and thus absorb such light. The absorption of this light acts as a marker of the degree of folding of the protein. CD is a valuable tool for showing changes in conformation. The technique can be used to study how the secondary structure of a protein changes by measuring the change in the absorption as a function of temperature. In this way, CD can reveal important thermodynamic information about the protein (such as the enthalpy and Gibbs free energy of denaturation) that cannot otherwise be easily obtained.

In certain embodiments, the melting temperature of the GCase variant may be increased by at least 2.0° C. compared with the melting temperature of human wild-type GCase. For example, the melting temperature may be increased by at least 2.0° C., at least 3.0° C., at least 4.0° C., at least 5.0° C., at least 10.0° C., at least 15.0° C., at least 20.0° C., at least 25.0° C., or at least 30.0° C. compared with the melting temperature of human wild-type GCase.

In certain embodiments, the biologically active variant of human wild-type glucocerebrosidase differs from human wild-type glucocerebrosidase by a single amino acid substitution at one or more positions selected from the group consisting of K321, H145, F316, and L317. Single amino acid substitution at a given position in a protein or polypeptide denotes the replacement of the single amino acid at that position with exactly one other amino acid. The variant may contain one single amino acid substitution, or may contain two or more single amino acid substitutions at respectively two or more positions. Single amino acid substitutions at K321, H145, F316, and/or L317 had been previously described to benefit the stability of GCase (see U.S. Pat. No. 8,962,564).

In certain preferred embodiments, the biologically active variant of human wild-type glucocerebrosidase differs from human wild-type glucocerebrosidase by a single amino acid substitution at K321, or at H145, or at K321 and H145.

In certain more preferred embodiments, the biologically active variant of human wild-type glucocerebrosidase differs from human wild-type glucocerebrosidase by K321N substitution, or by H145L substitution, or by K321N and H145L substitutions.

In certain embodiments, the glucocerebrosidase H145L/K321N variant comprises or consists of the amino acid sequence as set forth in SEQ ID NO: 3:

(SEQ ID NO: 3)
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSPPA
QNLLLKSYFSEEGIGYNIIRVPMASCDFSIRTYTYADTPDDFQLLNFSLP
EEDTKLKIPLIHRALQLAQRPVSLLASPWTSPTWLKTNGAVNGKGSLKGQ
PGDIYHQTWARYFVKFLDAYAEHKLQFWAVTAENEPSAGLLSGYPFQCLG
FTPEHQRDFIARDLGPTLANSTHHNVRLLMLDDQRLLLPHWAKVVLTDPE
AAKYVHGIAVHWYLDFLAPANATLGETHRLFPNTMLFASEACVGSKFWEQ
SVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALM
HPDGSAVVVVLNRSSKDVPLTIKDPAVGFLETISPGYSIHTYLWRRQ

In certain embodiments, the glucocerebrosidase H145L variant comprises or consists of the amino acid sequence as set forth in SEQ ID NO: 4:

(SEQ ID NO: 4)
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSPPA
QNLLLKSYFSEEGIGYNIIRVPMASCDFSIRTYTYADTPDDFQLLNFSLP
EEDTKLKIPLIHRALQLAQRPVSLLASPWTSPTWLKTNGAVNGKGSLKGQ
PGDIYHQTWARYFVKFLDAYAEHKLQFWAVTAENEPSAGLLSGYPFQCLG
FTPEHQRDFIARDLGPTLANSTHHNVRLLMLDDQRLLLPHWAKVVLTDPE
AAKYVHGIAVHWYLDFLAPAKATLGETHRLFPNTMLFASEACVGSKFWEQ
SVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALM
HPDGSAVVVVLNRSSKDVPLTIKDPAVGFLETISPGYSIHTYLWRRQ

In certain embodiments, the glucocerebrosidase K321N variant comprises or consists of the amino acid sequence as set forth in SEQ ID NO: 5:

(SEQ ID NO: 5)
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSPPA
QNLLLKSYFSEEGIGYNIIRVPMASCDFSIRTYTYADTPDDFQLHNFSLP
EEDTKLKIPLIHRALQLAQRPVSLLASPWTSPTWLKTNGAVNGKGSLKGQ
PGDIYHQTWARYFVKFLDAYAEHKLQFWAVTAENEPSAGLLSGYPFQCLG
FTPEHQRDFIARDLGPTLANSTHHNVRLLMLDDQRLLLPHWAKVVLTDPE
AAKYVHGIAVHWYLDFLAPANATLGETHRLFPNTMLFASEACVGSKFWEQ
SVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALM
HPDGSAVVVVLNRSSKDVPLTIKDPAVGFLETISPGYSIHTYLWRRQ

The glucocerebrosidase as intended herein may preferably be produced recombinantly. The term “recombinant” is generally used to indicate that the material (e.g., a nucleic acid, a genetic construct or a protein) has been altered by technical means (i.e., non-naturally) through human intervention. The term “recombinant nucleic acid” commonly refers to nucleic acids comprised of segments joined together using recombinant DNA technology. The term “recombinant protein or polypeptide” commonly refers to proteins or polypeptides that result from the expression of recombinant nucleic acid such as recombinant DNA.

For recombinant expression of the GCase, an expression cassette or an expression vector comprising a nucleic acid molecule encoding the GCase and a promoter operably linked to the nucleic acid molecule may be constructed. Preferably, the expression cassette or expression vector may be configured to effect expression of the GCase in a suitable host cell.

The terms “expression vector” or “vector” as used herein refers to nucleic acid molecules, typically DNA, to which nucleic acid fragments, preferably the recombinant nucleic acid molecule as defined herein, may be inserted and cloned, i.e., propagated. Hence, a vector will typically contain one or more unique restriction sites, and may be capable of autonomous replication in a defined host cell or vehicle organism such that the cloned sequence is reproducible. A vector may also preferably contain a selection marker, such as, e.g., an antibiotic resistance gene, to allow selection of recipient cells that contain the vector. Vectors may include, without limitation, plasmids, phagemids, bacteriophages, bacteriophage-derived vectors, PAC, BAC, linear nucleic acids, e.g., linear DNA, viral vectors, etc., as appropriate (see, e.g., Sambrook et al., 1989; Ausubel 1992). Expression vectors are generally configured to allow for and/or effect the expression of nucleic acids or ORFs introduced thereto in a desired expression system, e.g., in vitro, in a host cell, host organ and/or host organism. For example, expression vectors may advantageously comprise suitable regulatory sequences.

Factors of importance in selecting a particular vector include inter alia: choice of recipient host cell, ease with which recipient cells that contain the vector may be recognised and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A recombinant nucleic acid can be in introduced into the host cell in the form of an expression vector such as a plasmid, phage, transposon, cosmid or virus particle. The recombinant nucleic acid can be maintained extrachromosomally or it can be integrated into the cell chromosomal DNA. Expression vectors can contain selection marker genes encoding proteins required for cell viability under selected conditions (e.g., URA3, which encodes an enzyme necessary for uracil biosynthesis or TRP1, which encodes an enzyme required for tryptophan biosynthesis) to permit detection and/or selection of those cells transformed with the desired nucleic acids. Expression vectors can also include an autonomous replication sequence (ARS).

Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the host cell species to be transformed. A nucleotide sequence containing a gene of interest for expression is inserted in this vector between the first and second insertable DNA fragments, whether before or after the marker gene. Integrative vectors can be linearized prior to transformation to facilitate the integration of the nucleotide sequence of interest into the host cell genome.

As used herein, the term “promoter” refers to a DNA sequence that enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control expression of a coding sequence of interest, such as the nucleic acid molecule as defined herein.

The promotor may be a constitutive or inducible (conditional) promoter. A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues.

For example, the promoter may be a promoter for expression in a fungal cell, such as a Yarrowia lipolytica cell, e.g., a promoter from a suitable fungal species, such as Yarrowia lipolytica, Arxula adeninivorans, P. pastoris, or other suitable fungal species. Suitable fungal or yeast promoters include, e.g., ADC1, TPI1, ADH2, hp4d, TEF1, POX2, or Gal10 promoter. Preferably, the promoter is hp4d or POX2. More preferably, the promoter is hp4d. See, e.g., Guarente et al., 1982, Proc. Natl. Acad. Sci. USA 79(23):7410; Zhu and Zhang, 1999, Bioinformatics 15(7-8):608-611; or U.S. Pat. No. 6,265,185.

The glucocerebrosidase may be produced in any host cell system. Common host cell systems may include fungal cells, including yeast cells, animal cells, mammalian cells, including human cells and non-human mammalian cells. Such host cell systems may allow or may have been engineered or configured to allow for production of glycoproteins having an extent of glycan phosphorylation as required herein.

In certain embodiments, the host cell may be a fungal cell, including a yeast cell. In certain embodiments, the host cell may be a yeast cell. Fungal and yeast host cells include inter alia Yarrowia lipolytica, Arxula adeninivorans, Saccharomyces cerevisiae, Pichia pastoris, Pichia methanolica, Ogataea minuta, Kluyveromyces lactis, Schizosaccharomyces pombe, Hansenula polymorpha, or Aspergillus sp.

In certain embodiments, the host cell may be Yarrowia lipolytica or Arxula adeninivorans. Preferably, the host cell is Yarrowia lipolytica.

In certain embodiments, the host cell is a fungal cell genetically engineered to produce glucocerebrosidase. In particular embodiments, the host cell is a fungal cell genetically engineered to produce glucocerebrosidase comprising glycans at least 30% of which comprise at least one mannose-1-phospho-6-mannose moiety. In particular embodiments, the host cell is a fungal cell genetically engineered to produce glucocerebrosidase comprising glycans at least 10% of which comprise two mannose-1-phospho-6-mannose moieties. Such glycans may particularly include ManP-Man₈GlcNAc₂ and (ManP)₂-Man₈GlcNAc₂.

In certain embodiments, the host cell is a Yarrowia lipolytica cell genetically engineered to produce glucocerebrosidase. In particular embodiments, the host cell is a Yarrowia lipolytica cell genetically engineered to produce glucocerebrosidase comprising glycans at least 30% of which comprise at least one mannose-1-phospho-6-mannose moiety. In particular embodiments, the host cell is a Yarrowia lipolytica cell genetically engineered to produce glucocerebrosidase comprising glycans at least 10% of which comprise two mannose-1-phospho-6-mannose moieties. Such glycans may particularly include ManP-Man₈GlcNAc₂ and (ManP)₂-Man₈GlcNAc₂.

Preferably, the host cell, such as fungal cell, such as Yarrowia lipolytica cell, may comprise a deficiency in outer chain elongation of N-glycans activity, such as a deficiency in OCH1 activity. This abrogates the potential of synthesizing hyperglycosyl structures onto secreted glycoproteins. The main N-glycan on total extracellular protein is neutral Man₈GlcNAc₂. Preferably, the host cell, such as fungal cell, such as Yarrowia lipolytica cell, may comprise overexpression of a polypeptide capable of effecting mannosyl phosphorylation of N-glycans, such as MNN4 or PNO1. This promotes inclusion of mannose-1-phospho-6-mannose moieties in N-glycans. Particularly preferably, the host cell, such as fungal cell, such as Yarrowia lipolytica cell, comprises a deficiency in outer chain elongation of N-glycans activity and comprises overexpression of a polypeptide capable of effecting mannosyl phosphorylation of N-glycans. Particularly preferably, the host cell, such as fungal cell, such as Yarrowia lipolytica cell, comprises an OCH1 deficiency and overexpression of MNN4 or PNO1. This results in the conversion of almost all neutral N-glycans into structures containing one or two mannose-1-phospho-6-mannose moieties. The main N-glycans on total extracellular protein are ManP-Man₈GlcNAc₂ and (ManP)₂-Man₈GlcNAc₂. For example, MNN4 polypeptide from Yarrowia lipolytica, S. cerevisiae, Ogataea minuta, Pichia pastoris, or C. albicans, or PNO1 from P. pastoris, may be overexpressed in the fungal cell, preferably Yarrowia lipolytica cell. Preferably, MNN4 polypeptide from Yarrowia lipolytica may be overexpressed in the fungal cell, preferably Yarrowia lipolytica cell. An illustrative MNN4 polypeptide from Y. lipolytica has Genbank accession no: XM_503217.1. The aforementioned genetic modifications of Yarrowia lipolytica to produce glycoproteins with highly phosphorylated N-glycans, particularly with high proportion of ManP-Man₈GlcNAc₂ and (ManP)₂-Man₈GlcNAc₂ N-glycans, have been described in WO 2008/120107 and in Tiels et al. (Nat Biotechnol. 2012, vol. 30, 1225-31), incorporated by reference herein.

As mentioned, in phosphorylated N-glycans produced by fungal cells, such as by Yarrowia lipolytica, phosphate groups are capped with a mannose group, hence, the N-glycans comprise mannose-1-phospho-6-mannose moieties. To facilitate binding to mannose-6-phosphate receptor on mammalian cells, such as human cells, and subsequent transport to the interior of the cells and eventually to lysosomes, fungal cell-produced glycoproteins containing phosphorylated N-glycans may need to be uncapped. In this connection, “uncapped” particularly means that the phosphate group in the phospho-6-mannose moiety is not covalently linked to another moiety, e.g., to the mannos-1-yl moiety, and “uncapping” particularly refers to removing the mannos-1-yl residue, thereby exposing the phosphate moiety. Where an N-glycan contains more than one phosphate groups, the N-glycan may be denoted as “uncapped” if at least one of said phosphate groups is uncapped. Preferably, both said phosphate groups may be uncapped.

Further, phosphorylated N-glycans produced by fungal cells, such as by Yarrowia lipolytica, are of high mannose type, and typically contain one or more mannose residues bound to the mannose underlying the mannose to which the phosphate group is bound (i.e., underlying the mannose-1-phospho-6-mannose moiety). By means of an example, in the aforementioned case of a Yarrowia lipolytica cell deficient in OCH1 and overexpressing MNN4 or PNO1, such N-glycans may be ManP-Man₈GlcNAc₂ and (ManP)₂-Man₈GlcNAc₂ N-glycans. Such structures may need to be demannosylated. In this connection, “demannosylated” may refer to at least the hydrolysis of terminal alpha-1,2 mannose moieties of phosphate-containing N-glycans, including the terminal alpha-1,2-mannose when the underlying mannose is phosphorylated. Hence, this results in the mannose containing the phosphate at the 6 position becoming the terminal mannose. In certain embodiments, “demmanosylated” may refer to hydrolysis of terminal alpha-1,2 mannose, alpha-1,3 mannose and/or (preferably “and”) alpha-1,6 mannose linkages or moieties of phosphate-containing N-glycans. More particularly, in a phosphorylated (mono- or di-phosphorylated)N-glycan, demannosylation may include hydrolysis of the non-phosphorylated arm of the N-glycan and hydrolysis of the terminal alpha-1,2-mannose when the underlying mannose is phosphorylated. In such case, final hydrolysis products of demannosylation may be selected from the group comprising, consisting essentially of or consisting of PMan₃GlcNAc₂ and P₂Man₅GlcNAc₂ (where uncapping has also been performed). Demannosylated N-glycans containing uncapped phosphate group(s) bind substantially better to mannose-6-phosphate receptors on mammalian cells than non-demannosylated N-glycans containing uncapped phosphate group(s), thereby increasing the efficiency with which the GCase is transported to the interior of mammalian cells and eventually to the lysosome.

Hence, in certain embodiments, the glucocerebrosidase is obtainable or obtained by uncapping and demannosylation of glucocerebrosidase recombinantly expressed by a fungal cell genetically engineered to produce glucocerebrosidase, in particular genetically engineered to produce glucocerebrosidase comprising glycans at least 30% of which comprise at least one mannose-1-phospho-6-mannose moiety.

In further embodiments, the glucocerebrosidase is obtainable or obtained by uncapping and demannosylation of glucocerebrosidase recombinantly expressed by a Yarrowia lipolytica cell genetically engineered to produce glucocerebrosidase, in particular genetically engineered to produce glucocerebrosidase comprising glycans at least 30% of which comprise at least one mannose-1-phospho-6-mannose moiety.

In further embodiments, the glucocerebrosidase is obtainable or obtained by uncapping and demannosylation of glucocerebrosidase recombinantly expressed by a fungal cell genetically engineered to produce glucocerebrosidase, in particular genetically engineered to produce glucocerebrosidase comprising glycans at least 10% of which comprise two mannose-6-phosphate moieties.

In further embodiments, the glucocerebrosidase is obtainable or obtained by uncapping and demannosylation of glucocerebrosidase recombinantly expressed by a Yarrowia lipolytica genetically engineered to produce glucocerebrosidase, in particular genetically engineered to produce glucocerebrosidase comprising glycans at least 10% of which comprise two mannose-6-phosphate moieties.

Glycoproteins containing a phosphorylated N-glycan can be demannosylated, and glycoproteins containing a phosphorylated N-glycan containing a mannose-1-phospho-6-mannose linkage or moiety can be uncapped and demannosylated by contacting the glycoprotein with a mannosidase capable of (i) hydrolyzing a mannose-1-phospho-6-mannose linkage or moiety to mannose-6-phosphate and (ii) hydrolyzing a terminal alpha-1,2 mannose, alpha-1,3 mannose and/or alpha-1,6 mannose linkage or moiety. Non-limiting examples of such mannosidases include a Canavalia ensiformis (Jack bean) mannosidase and a Yarrowia lipolytica mannosidase (e.g., AMS1). Both the Jack bean and AMS1 mannosidase are family 38 glycoside hydrolases.

The Jack bean mannosidase is commercially available, for example, from Sigma-Aldrich (St. Louis, Mo.) as an ammonium sulphate suspension (Catalog No. M7257) and a proteomics grade preparation (Catalog No. M5573). Such commercial preparations can be further purified, for example, by gel filtration chromatography to remove contaminants such as phosphatases.

The Yarrowia lipolytica AMS1 mannosidase can be recombinantly produced. The amino acid sequence of the AMS1 polypeptide is set forth in WO 2013/136189 as SEQ ID NO: 5.

In some embodiments, the uncapping and demannosylating steps are catalysed by two different enzymes. For example, uncapping of a mannose-1-phospho-6 mannose linkage or moiety can be performed using a mannosidase from Cellulosimicrobium cellulans (e.g., CcMan5). The nucleotide sequence encoding the CcMan5 polypeptide is set forth in WO 2013/136189 as SEQ ID NO: 2. The amino acid sequence of the CcMan5 polypeptide containing a signal sequence is set forth in WO 2013/136189 as SEQ ID NO: 3. The amino acid sequence of the CcMan5 polypeptide without signal sequence is set forth in WO 2013/136189 as SEQ ID NO: 4. In some embodiments, a biologically active fragment of the CcMan5 polypeptide is used. For example, a biologically active fragment can include residues 1-774 of the amino acid sequence set forth in WO 2013/136189 as SEQ ID NO: 4. See also WO 2011/039634. The CcMan5 mannosidase is a family 92 glycoside hydrolase.

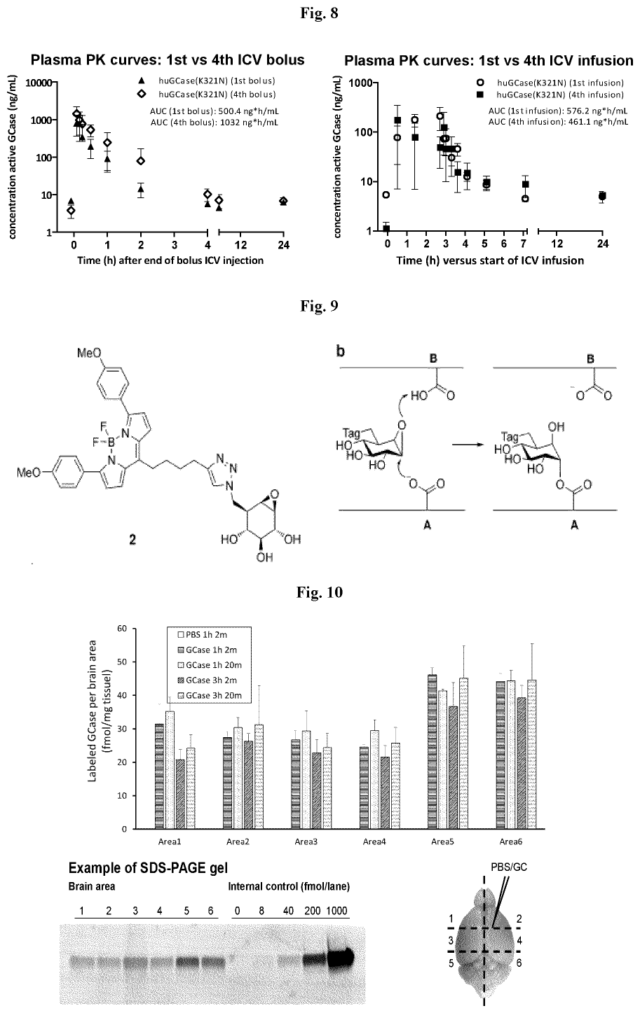

Demannosylation of an uncapped glycoprotein can be catalyzed using a mannosidase from Aspergillus satoi (As) (also known as Aspergillus phoenicis) or a mannosidase from Cellulosimicrobium cellulans (e.g., CcMan₄). The Aspergillus satoi mannosidase is a family 47 glycoside hydrolase and the CcMan4 mannosidase is a family 92 glycoside hydrolase. The amino acid sequence of the Aspergillus satoi mannosidase is set forth in WO 2013/136189 as SEQ ID NO: 6 and in Genbank Accession No. BAA08634.1. The amino acid sequence of the CcMan4 polypeptide is set forth in FIG. 8 of WO 2013/136189.

Demannosylation of an uncapped glycoprotein also can be catalyzed using a mannosidase from the family 38 glycoside hydrolases such as a Canavalia ensiformis (Jack bean) mannosidase or a Yarrowia lipolytica mannosidase (e.g., AMS1). For example, CcMan5 can be used to uncap a mannose-1-phospho-6 mannose moiety on a glycoprotein (or molecular complex of glycoproteins) and the Jack bean mannosidase can be used to demannosylate the uncapped glycoprotein (or molecular complex of glycoproteins).

To produce demannosylated glycoproteins, or uncapped and demannosylated glycoproteins, a glycoprotein containing a mannose-1-phospho-6 mannose linkage or moiety is contacted under suitable conditions with a suitable mannosidase(s) and/or a cell lysate containing a suitable native or recombinantly produced mannosidase(s). Suitable mannosidases are described above. The cell lysate can be from any genetically engineered cell, including a fungal cell, a plant cell, or animal cell. Non-limiting examples of animal cells include nematode, insect, plant, bird, reptile, and mammals such as a mouse, rat, rabbit, hamster, gerbil, dog, cat, goat, pig, cow, horse, whale, monkey, or human.

Upon contacting the glycoprotein with the purified mannosidases and/or cell lysate, the mannose-1-phospho-6-mannose linkage or moiety can be hydrolyzed to phospho-6-mannose and the terminal alpha-1,2 mannose, alpha-1,3 mannose and/or (preferably “and”) alpha-1,6 mannose linkage or moiety of such a phosphate containing glycan can be hydrolyzed to produce an uncapped and demannosylated glycoprotein. In some embodiments, one mannosidase is used that catalyzes both the uncapping and demannosylating steps. In some embodiments, one mannosidase is used to catalyze the uncapping step and a different mannosidase is used to catalyze the demannosylating step. Following processing by the mannosidase, the glycoprotein can be isolated.

The glucocerebrosidase as intended herein may be provided in any suitable or operable form or format. The glucocerebrosidase may be isolated, hence, existing or provided in separation from one or more other components of its natural environment. The glucocerebrosidase may be recombinantly produced. By means of an example, a glucocerebrosidase preparation may comprise, consist essentially of, or consist of the purified glucocerebrosidase. The term “purified” in this context does not require absolute purity. Instead, it denotes that the thing that has been purified is in a discrete environment in which its abundance relative to other components is greater than in the original material. A discrete environment denotes a single medium, such as for example a single solution, gel, precipitate, lyophilisate, etc. Subsequent to purification, the glucocerebrosidase may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951 J Biol Chem 193:265), optionally as described by Hartree 1972 Anal Biochem 48:422-427. Purity of peptides, polypeptides, or proteins may be determined by SDS-PAGE under reducing or non-reducing conditions using Coomassie blue or, preferably, silver stain. In certain embodiments, the glucocerebrosidase may be provided in a lyophilised form. In certain embodiments, the glucocerebrosidase may be provided in an aqueous solution.

The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. By means of an example, a glucocerebrosidase composition may comprise the glucocerebrosidase in combination with one or more other substances. For example, a glucocerebrosidase composition may be obtained by combining, such as admixing, the glucocerebrosidase with said one or more other substances. In certain embodiments, the present compositions may be configured as pharmaceutical compositions. Pharmaceutical compositions typically comprise one or more pharmacologically active ingredients (chemically and/or biologically active materials having one or more pharmacological effects) and one or more pharmaceutically acceptable carriers. Compositions as typically used herein may be liquid, semisolid or solid, and may include solutions or dispersions.

A further aspect provides a pharmaceutical composition comprising the glucocerebrosidase preparation or composition as taught herein.

The terms “pharmaceutical composition” and “pharmaceutical formulation” may be used interchangeably. The pharmaceutical compositions as taught herein may comprise in addition to the herein particularly specified components one or more pharmaceutically acceptable excipients. Suitable pharmaceutical excipients depend on the dosage form and identities of the active ingredients and can be selected by the skilled person (e.g., by reference to the Handbook of Pharmaceutical Excipients 7^{th} Edition 2012, eds. Rowe et al.). As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the GCase. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability.

The pharmaceutical formulations may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5. Preferably, to increase stability and storage time of the GCase, pH may be slightly acidic. In certain embodiments, the pharmaceutical composition has pH between about 5.0 and about 6.9, such as about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9. In certain embodiments, the pharmaceutical composition has pH of about 6.4 to 6.9, preferably of about 6.6. The preparation of such pharmaceutical formulations is within the ordinary skill of a person skilled in the art.

Administration of the pharmaceutical composition can be systemic or local (topical). Pharmaceutical compositions can be formulated such that they are suitable for parenteral and/or enteral administration. Specific administration modalities include subcutaneous, intravenous, intramuscular, intraperitoneal, transdermal, intracerebroventricular (ICV), intrathecal, oral, rectal, buccal, topical, nasal, ophthalmic, intra-articular, intra-arterial, sub-arachnoid, bronchial, lymphatic, vaginal, and intra-uterine administration.

In certain preferred embodiments, the administration may be intravenous (IV), such as IV infusion or injection. For IV administration, the composition may have comparatively lower pH, such as pH between about 5.0 and 6.0, e.g., about 5.5 (e.g., using citrate buffer). In certain preferred embodiments, the administration may be intracerebroventricular (ICV), such as ICV infusion or injection. For ICV administration, the composition may have pH comparatively closer to physiological pH, such as pH between 6.1 and 7.4, preferably between 6.4 and 6.9, e.g., about 6.6.

In certain embodiments, particularly for ICV or intrathecal administration, the glucocerebrosidase may be formulated with artificial cerebrospinal fluid (aCFS).

Compositions denoted as artificial cerebrospinal fluid (aCSF) encompass any multivalent physiological ion solutions designed to mimic physiological cerebrospinal fluid. aCSF may illustratively contain 127 mM NaCl, 1.0 mM KCl, 1.2 mM KH₂PO₄, 26 mM NaHCO₃, 10 mM D-glucose, 2.4 mM CaCl₂, and 1.3 mM MgCl₂. aCSF may illustratively contain 119 mM NaCl, 26.2 mM NaHCO₃, 2.5 mM KCl, 1 mM NaH₂PO₄, 1.3 mM MgCl₂, 10 mM glucose, and 2.5-mM CaCl₂. Electrolyte concentrations in aCSF may illustratively be 150 mM Na⁺, 3.0 mM K %, 1.4 mM Ca²⁺, 0.8 mM Mg²⁺, 1.0 mM phosphate, and 155 mM Cl⁻. In certain preferred embodiments, the aCSF may contain 148 mM NaCl, 3 mM KCl, 1.4 mM CaCl₂.2H₂O, 0.8 mM MgCl₂.6H₂O, 0.465 mM Na₂HPO₄.7H₂O, and 0.535 mM NaH₂PO₄.H₂O. The pH of aCSF is optionally at or between 3 and 10. In certain embodiments, the pH may be between 6.1 and 7.4, preferably between 6.4 and 6.9, e.g., about 6.6.

Several studies have reported that human GCase can be stabilised at neutral pH when bound by a pharmacological chaperone such as isofagomine or ambroxol (Kornhaber et al., 2008, Chembiochem., vol. 9, 2643-2649; Maegawa et al., 2009, Journal of Biological Chemistry, vol. 284, 23502-23516), and such pharmacological chaperone(s) may be included in the present compositions.

A further aspect provides the glucocerebrosidase preparation or composition or pharmaceutical composition as taught herein for use in therapy. A related aspect provides a method for treating a subject in need thereof, the method comprising administering to the subject a prophylactically or therapeutically effective amount of the glucocerebrosidase preparation or composition or the pharmaceutical composition as taught herein.

Certain embodiments provide the glucocerebrosidase preparation or composition or pharmaceutical composition as taught herein for use in a method of treating a disease characterised by glucocerebrosidase deficiency. A related aspect provides a method for treating a disease characterised by glucocerebrosidase deficiency in a subject in need thereof, the method comprising administering to the subject a prophylactically or therapeutically effective amount of the glucocerebrosidase preparation or composition or pharmaceutical composition as taught herein.

Diseases characterised by glucocerebrosidase deficiency broadly encompass any diseases, disorders or pathological conditions in which a reduction or decrease in or abolishment of glucocerebrosidase activity in cells compared to a healthy or physiological state causes, contributes to, or is associated with the disease, disorder or pathological condition. By means of an example, such reduction or decrease in or abolishment of glucocerebrosidase activity may be a consequence of one or more mutations, particularly one or more loss-of-function mutations, in the gene encoding native glucocerebrosidase (e.g., GBA1 in humans). Without limitation, such mutations may cause reduced transcription of the GBA1 gene, may interfere with the processing, stability, trafficking or translation of the GBA1 transcript, or may alter the expression, processing, trafficking, structure and/or activity of the native glucocerebrosidase protein. Without limitation, mutations in the glucocerebrosidase protein may include insertions, deletions or substitutions, including frameshift mutations leading to truncated forms of the protein, and point mutations leading to substitutions of one or more amino acids in the protein. Alternatively, such reduction or decrease in or abolishment of glucocerebrosidase activity may not be due to a mutation in the gene encoding native glucocerebrosidase, but may have other causes which impact glucocerebrosidase.

In certain embodiments, the disease is Gaucher disease. The term is well established in the medical practice and inter alia includes any and all clinically recognised subtypes of Gaucher disease, such as in particular type I (non-neuropathic), type II (acute infantile neuropathic) and type III (chronic neuropathic).

In certain embodiments, the disease is non-neuronopathic Gaucher disease. In certain embodiments, systemic, such as IV, administration may be preferred for non-neuronopathic Gaucher disease forms.

In certain embodiments, the disease is neuronopathic Gaucher disease. In certain embodiments, ICV administration may be preferred for neuronopathic Gaucher disease forms. In certain embodiments, the disease is neuronopathic Gaucher disease type 2 (GD2), type 3 (GD3), or perinatal lethal (GDPL).

In certain embodiments, the disease is glucocerebrosidase-associated alpha-synucleinopathy. In this context, glucocerebrosidase-associated refers to the disease being characterised by (e.g., caused by, contributed to, or associated with) glucocerebrosidase deficiency as explained above.

Synucleinopathies or α-synucleinopathies broadly encompass a group of diseases affecting the nervous system, more particularly neurodegenerative diseases, characterised by the abnormal accumulation of aggregates of α-synuclein protein in neurons, nerve fibres or glial cells. In certain embodiments, ICV administration may be preferred for glucocerebrosidase-associated alpha-synucleinopathies.

In certain embodiments, the glucocerebrosidase-associated alpha-synucleinopathy is parkinsonism, Parkinson's disease, Multiple System Atrophy (MSA), or Lewis Body Dementia (LBD).

Reference to “therapy” or “treatment” broadly encompasses both curative and preventative treatments, and the terms may particularly refer to the alleviation or measurable lessening of one or more symptoms or measurable markers of a pathological condition such as a disease or disorder.

The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention.

The terms “subject”, “individual” or “patient” are used interchangeably throughout this specification, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, buffalo, deer, horses, mules and donkeys, and non-mammals such as birds, chickens, including chickens, quails, turkeys, partridges, pheasants, ducks, geese, or swans, amphibians, reptiles etc. The term “mammal” includes any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, gerbils, cattle, cows, sheep, horses, pigs and primates, e.g., monkeys and apes (e.g., chimpanzee, baboon, or monkey). In certain embodiments, the subject is a non-human mammal. Particularly preferred are human subjects including both genders and all age categories thereof. Preferably, GCase for administration to human subjects may be human wild-type GCase or a variant or fragment thereof as described herein. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. The term “prophylactically effective amount” generally denotes an amount sufficient to elicit the preventative effect, such as inhibition or delay of the onset of a disease, in a subject that is being sought by the medical practitioner, in either a single or multiple doses. Appropriate prophylactically or therapeutically effective doses of the present compositions or components of the kits-of-parts may be determined by a qualified physician with due regard to the nature and severity of the disease, and the age and condition of the patient. The effective amount of the compositions or components of the kits-of-parts described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

The dosage or amount of the GCase polypeptide as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent(s) of the invention. In order to optimize therapeutic efficacy, the GCase as described herein can be first administered at different dosing regimens. Typically, levels of the GCase in a tissue can be monitored using appropriate screening assays as part of a clinical testing procedure, e.g., to determine the efficacy of a given treatment regimen. The frequency of dosing is within the skills and clinical judgement of medical practitioners (e.g., doctors or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the GCase polypeptide as described herein can be determined by known pharmaceutical procedures in, for example, cell cultures or experimental animals. These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects (e.g., human patients). The dosage of such pharmaceutical compositions lies generally within a range of circulating concentrations that include the ED₅₀ with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

Without limitation, depending on the type and severity of the disease, a typical dosage (e.g., a typical daily dosage or a typical intermittent dosage, e.g., a typical dosage for every two days, every three days, every four days, every five days, every six days, every week, every 1.5 weeks, every two weeks, every three weeks, every month, or other) of the GCase polypeptide as taught herein may range from about 10 μg/kg to about 100 mg/kg body weight of the subject, per dose, depending on the factors mentioned above, e.g., may range from about 100 μg/kg to about 10 mg/kg body weight of the subject, per dose, or from about 200 μg/kg to about 2 mg/kg body weight of the subject, per dose, e.g., may be about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, or about 2.0 mg/kg body weight of the subject, per dose, daily or intermittently, preferably intermittently, more preferably every week, even more preferably every other week, yet more preferably every month or even less frequently. By means of example and without limitation, the GCase may be administered at about 0.5 mg/kg, or at about 0.6 mg/kg, or at about 0.7 mg/kg, or at about 0.8 mg/kg, or at about 0.9 mg/kg, or at about 1.0 mg/kg, or at about 1.5 mg/kg, or at about 2.0 mg/kg, or at about 2.5 mg/kg, or at about 3.0 mg/kg, or at about 3.5 mg/kg, or at about 4.0 mg/kg, e.g., at about 0.6-0.8 mg/kg or at about 3-4 mg/kg, preferably bi-weekly.

When ICV-administered, the GCase as taught herein may be administered at between 5 and 30 mg/100 g brain weight, such as between 10 and 20 mg/100 g brain weight, for example at about 10, 11, 12, 13, 14, 15, 16, 17, 18, 19 or 20 mg/100 g brain weight. By means of an example, the approximate weight of the brain of 2-3 year-old children is 1.2 kg, and the GCase as taught herein may be administered to such subjects at between 60 mg and 360 mg per dose, such as between 120 mg and 280 mg per dose, such as at about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, or about 280 mg per dose, such as preferably between 180 mg and 240 mg per dose, or more preferably between 200 mg and 220 mg per dose, such as particularly preferably at about 210 mg per dose. Such administration may be weekly, bi-weekly, or monthly, preferably weekly.

In certain embodiments, the glucocerebrosidase preparation or composition or pharmaceutical composition as taught herein is administered systemically. In certain embodiments, the glucocerebrosidase preparation or composition or pharmaceutical composition as taught herein is administered intravenously (IV), such as by IV injection or infusion. Such systemic, in particular IV administration, may be particularly but without limitation suited for non-neuronopathic forms of Gaucher disease.

In certain embodiments, the glucocerebrosidase preparation or composition or pharmaceutical composition as taught herein is administered into the central nervous system (CNS). CNS administration may be particularly preferred for neuronopathic Gaucher disease forms and for GCase-associated α-synucleinopathies.

In certain embodiments, the glucocerebrosidase preparation or composition or pharmaceutical composition as taught herein is administered intracerebroventricularly (ICV), intrathecally or intraparenchymally (to the CNS), preferably ICV or intrathecally, more preferably ICV, such as ICV injection or infusion. ICV administration, such as ICV infusion or injection, may preferably be unilateral, preferably may be directed to either the right or the left lateral ventricle. Repeated or chronic ICV, intrathecal or intraparenchymal administration may for example be facilitated by a cannula or catheter implanted to the target ventricle. Such systems are known in the art, for example from US 2005/0208090.

In certain embodiments, the disease is neuronopathic Gaucher disease or glucocerebrosidase-associated alpha-synucleinopathy and the glucocerebrosidase preparation or composition or pharmaceutical composition as taught herein is administered intracerebroventricularly (ICV) or intrathecally.

In certain embodiments, the disease is neuronopathic Gaucher disease or glucocerebrosidase-associated α-synucleinopathy and the glucocerebrosidase preparation or composition or pharmaceutical composition as taught herein is administered intracerebroventricularly (ICV).

The present application also provides aspects and embodiments as set forth in the following numbered Statements:

Statement 1. A glucocerebrosidase preparation or a composition comprising glucocerebrosidase, wherein at least 30% of glycans comprised by the glucocerebrosidase comprise at least one mannose-6-phosphate moiety.

Statement 2. The preparation or composition according to Statement 1, wherein at least 40%, or at least 50%, or at least 60%, or at least 70%, or at least 80%, or at least 90%, or at least 95%, or at least 98%, or at least 99%, or substantially all of the glycans comprised by the glucocerebrosidase comprise at least one mannose-6-phosphate moiety.

Statement 3. The preparation or composition according to Statement 1 or 2, wherein at least some of the mannose-6-phosphate moiety-comprising glycans comprise two mannose-6-phosphate moieties.

Statement 4. The preparation or composition according to any one of Statements 1 to 3, wherein at least 5%, or at least 10%, or at least 15%, or at least 20%, or at least 25%, or at least 30%, or at least 35%, or at least 40%, or at least 45% of the mannose-6-phosphate moiety-comprising glycans comprise two mannose-6-phosphate moieties.

Statement 5. The preparation or composition according to any one of Statements 1 to 4, wherein at least 40% of the glucocerebrosidase molecules are glycosylated.

Statement 6. The preparation or composition according to any one of Statements 1 to 5, wherein at least 50%, or at least 60%, or at least 70%, or at least 80%, or at least 90%, or at least 95%, or at least 98%, or at least 99%, or substantially all of the glucocerebrosidase molecules are glycosylated.

Statement 7. The preparation or composition according to any one of Statements 1 to 6, wherein the glucocerebrosidase is human wild-type glucocerebrosidase, or a biologically active variant or fragment of human wild-type glucocerebrosidase.

Statement 8. The preparation or composition according to Statement 7, wherein the biologically active variant of human wild-type glucocerebrosidase displays at least 90% sequence identity to human wild-type glucocerebrosidase, such as at least 95% or at least 98% or at least 99% sequence identity to human wild-type glucocerebrosidase.

Statement 9. The preparation or composition according to Statement 7 or 8, wherein the biologically active variant of human wild-type glucocerebrosidase has increased stability and/or specificity relative to human wild-type glucocerebrosidase.

Statement 10. The preparation or composition according to any one of Statements 7 to 9, wherein the biologically active variant of human wild-type glucocerebrosidase differs from human wild-type glucocerebrosidase by a single amino acid substitution at one or more positions selected from the group consisting of K321, H145, F316, and L317.

Statement 11. The preparation or composition according to any one of Statements 7 to 10, wherein the biologically active variant of human wild-type glucocerebrosidase differs from human wild-type glucocerebrosidase by a single amino acid substitution at K321, or at H145, or at K321 and H145.

Statement 12. The preparation or composition according to any one of Statements 7 to 11, wherein the biologically active variant of human wild-type glucocerebrosidase differs from human wild-type glucocerebrosidase by K321N substitution, or by H145L substitution, or by K321N and H145L substitutions.

Statement 13. The preparation or composition according to any one of Statements 1 to 12, wherein the mannose of the mannose-6-phosphate moiety is a terminal mannose.

Statement 14. The preparation or composition according to any one of Statements 1 to 13, wherein the mannose-6-phosphate moiety-comprising glycans are each independently selected from the group comprising or consisting of PMan₇GlcNAc₂, PMan₆GlcNAc₂, PMan₅GlcNAc₂, PMan₄GlcNAc₂, PMan₃GlcNAc₂, P₂Man₆GlcNAc₂, and P₂Man₅GlcNAc₂.

Statement 15. The preparation or composition according to any one of Statements 1 to 14, wherein the mannose-6-phosphate moiety-comprising glycans are each independently selected from the group comprising or consisting of PMan₅GlcNAc₂, PMan₄GlcNAc₂, PMan₃GlcNAc₂, P₂Man₆GlcNAc₂, and P₂Man₅GlcNAc₂.

Statement 16. The preparation or composition according to any one of Statements 1 to 15, wherein the mannose-6-phosphate moiety-comprising glycans are each independently selected from the group comprising or consisting of PMan₃GlcNAc₂ and P₂Man₅GlcNAc₂.

Statement 17. The preparation or composition according to any one of Statements 1 to 16, wherein the glucocerebrosidase is obtainable or obtained by uncapping and demannosylation of glucocerebrosidase recombinantly expressed by a fungal cell genetically engineered to produce glucocerebrosidase, in particular genetically engineered to produce glucocerebrosidase comprising glycans at least 30% of which comprise at least one mannose-1-phospho-6-mannose moiety.

Statement 18. The preparation or composition according to any one of Statements 1 to 17, wherein the glucocerebrosidase is obtainable or obtained by uncapping and demannosylation of glucocerebrosidase recombinantly expressed by a Yarrowia lipolytica cell genetically engineered to produce glucocerebrosidase, in particular genetically engineered to produce glucocerebrosidase comprising glycans at least 30% of which comprise at least one mannose-1-phospho-6-mannose moiety.

Statement 19. A pharmaceutical composition comprising the glucocerebrosidase preparation or composition according to any one of Statements 1 to 18.

Statement 20. The pharmaceutical composition according to Statement 19, wherein the glucocerebrosidase is formulated with artificial cerebrospinal fluid (aCFS).

Statement 21. The pharmaceutical composition according to any one of Statements 19 or 20, wherein the pharmaceutical composition has pH of about 6.4 to 6.9, preferably of about 6.6.

Statement 22. The glucocerebrosidase preparation or composition according to any one of Statements 1 to 18 or the pharmaceutical composition according to any one of Statements 19 to 21, for use in therapy; or a method for treating a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 23. The glucocerebrosidase preparation or composition according to any one of Statements 1 to 18 or the pharmaceutical composition according to any one of Statements 19 to 21 for use in a method of treating a disease characterised by glucocerebrosidase deficiency; or a method for treating a disease characterised by glucocerebrosidase deficiency in a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 24. The glucocerebrosidase preparation or composition according to any one of Statements 1 to 18 or the pharmaceutical composition according to any one of Statements 19 to 21 for use in a method of treating Gaucher disease; or a method for treating Gaucher disease in a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 25. The glucocerebrosidase preparation or composition according to any one of Statements 1 to 18 or the pharmaceutical composition according to any one of Statements 19 to 21 for use in a method of treating non-neuronopathic Gaucher disease; or a method for treating non-neuronopathic Gaucher disease in a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 26. The glucocerebrosidase preparation or composition according to any one of Statements 1 to 18 or the pharmaceutical composition according to any one of Statements 19 to 21 for use in a method of treating neuronopathic Gaucher disease; or a method for treating neuronopathic Gaucher disease in a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 27. The glucocerebrosidase preparation or composition or pharmaceutical composition for use according to Statement 26 or the method according to Statement 26, wherein the neuronopathic Gaucher disease is type 2 (GD2), type 3 (GD3), or perinatal lethal (GDPL).

Statement 28. The glucocerebrosidase preparation or composition according to any one of Statements 1 to 18 or the pharmaceutical composition according to any one of Statements 19 to 21 for use in a method of treating glucocerebrosidase-associated alpha-synucleinopathy; or a method for treating glucocerebrosidase-associated alpha-synucleinopathy in a subject in need thereof, the method comprising administering to the subject a prophylactically or therapeutically effective amount of the glucocerebrosidase preparation or composition according to any one of Statements 1 to 18 or the pharmaceutical composition according to any one of Statements 19 to 21.

Statement 29. The glucocerebrosidase preparation or composition or pharmaceutical composition for use according to Statement 28 or the method according to Statement 28, wherein the glucocerebrosidase-associated alpha-synucleinopathy is parkinsonism, Parkinson's disease, Multiple System Atrophy (MSA), or Lewis Body Dementia (LBD).

Statement 30. The glucocerebrosidase preparation or composition or pharmaceutical composition for use according to any one of Statements 22 to 29, or the method according to any one of Statements 22 to 29, wherein the preparation or composition or pharmaceutical composition is administered systemically.

Statement 31. The glucocerebrosidase preparation or composition or pharmaceutical composition for use according to any one of Statements 22 to 30, or the method according to any one of Statements 22 to 30, wherein the preparation or composition or pharmaceutical composition is administered intravenously (IV).

Statement 32. The glucocerebrosidase preparation or composition or pharmaceutical composition for use according to any one of Statements 22 to 30, or the method according to any one of Statements 22 to 30, wherein the preparation or composition or pharmaceutical composition is administered into the central nervous system.

Statement 33. The glucocerebrosidase preparation or composition or pharmaceutical composition for use according to any one of Statements 22 to 30, or the method according to any one of Statements 22 to 30, wherein the preparation or composition or pharmaceutical composition is administered intracerebroventricularly (ICV) or intrathecally.

Statement 34. The glucocerebrosidase preparation or composition according to any one of Statements 1 to 18 or the pharmaceutical composition according to any one of Statements 19 to 21 for use in a method of treating neuronopathic Gaucher disease or glucocerebrosidase-associated alpha-synucleinopathy by intracerebroventricular (ICV) or intrathecal administration; or a method for treating neuronopathic Gaucher disease or glucocerebrosidase-associated alpha-synucleinopathy in a subject in need thereof, the method comprising intracerebroventricularly (ICV) or intrathecally administering to the subject a prophylactically or therapeutically effective amount of the glucocerebrosidase preparation or composition according to any one of Statements 1 to 18 or the pharmaceutical composition according to any one of Statements 19 to 21.

Statement 35. The glucocerebrosidase preparation or composition according to any one of Statements 1 to 18 or the pharmaceutical composition according to any one of Statements 19 to 21 for use in a method of treating neuronopathic Gaucher disease or glucocerebrosidase-associated alpha-synucleinopathy by intracerebroventricular (ICV) administration; or a method for treating neuronopathic Gaucher disease or glucocerebrosidase-associated alpha-synucleinopathy in a subject in need thereof, the method comprising intracerebroventricularly (ICV) administering to the subject a prophylactically or therapeutically effective amount of the glucocerebrosidase preparation or composition according to any one of Statements 1 to 18 or the pharmaceutical composition according to any one of Statements 19 to 21.

Statement 1*. A glucocerebrosidase preparation or a composition comprising glucocerebrosidase, wherein at least 10% of glycans comprised by the glucocerebrosidase comprise two mannose-6-phosphate moieties.

Statement 2*. The preparation or composition according to Statement 1*, wherein at least 15%, or at least 20%, or at least 25%, or at least 30%, or at least 35%, or at least 40%, or at least 45% of the glycans comprised by the glucocerebrosidase comprise two mannose-6-phosphate moieties.

Statement 3*. The preparation or composition according to Statement 1*, wherein more than 10% of glycans comprised by the glucocerebrosidase comprise at least one mannose-6-phosphate moiety.

Statement 4*. The preparation or composition according to Statement 3*, wherein at least 15%, or at least 20%, or at least 25%, or at least 30%, or at least 35%, or at least 40%, or at least 45% of the glycans comprised by the glucocerebrosidase comprise two mannose-6-phosphate moieties, and wherein, respectively, more than 15%, or more than 20%, or more than 25%, or more than 30%, or more than 35%, or more than 40%, or more than 45% of glycans comprised by the glucocerebrosidase comprise at least one mannose-6-phosphate moiety.

Statement 5*. The preparation or composition according to Statement 3*, wherein at least 20%, or at least 30%, or at least 40%, or at least 50%, or at least 60%, or at least 70%, or at least 80%, or at least 90%, or at least 95%, or at least 98%, or at least 99%, or substantially all of the glycans comprised by the glucocerebrosidase comprise at least one mannose-6-phosphate moiety.

Statement 6*. The preparation or composition according to any one of Statements 1* to 5*, wherein at least 40% of the glucocerebrosidase molecules are glycosylated.

Statement 7*. The preparation or composition according to any one of Statements 1* to 6*, wherein at least 50%, or at least 60%, or at least 70%, or at least 80%, or at least 90%, or at least 95%, or at least 98%, or at least 99%, or substantially all of the glucocerebrosidase molecules are glycosylated.

Statement 8*. The preparation or composition according to any one of Statements 1* to 7*, wherein the glucocerebrosidase is human wild-type glucocerebrosidase, or a biologically active variant or fragment of human wild-type glucocerebrosidase.

Statement 9*. The preparation or composition according to Statement 8*, wherein the biologically active variant of human wild-type glucocerebrosidase displays at least 90% sequence identity to human wild-type glucocerebrosidase, such as at least 95% or at least 98% or at least 99% sequence identity to human wild-type glucocerebrosidase.

Statement 10*. The preparation or composition according to Statement 8* or 9*, wherein the biologically active variant of human wild-type glucocerebrosidase has increased stability and/or specificity relative to human wild-type glucocerebrosidase.

Statement 11*. The preparation or composition according to any one of Statements 8* to 10*, wherein the biologically active variant of human wild-type glucocerebrosidase differs from human wild-type glucocerebrosidase by a single amino acid substitution at one or more positions selected from the group consisting of K321, H145, F316, and L317.

Statement 12*. The preparation or composition according to any one of Statements 8* to 11*, wherein the biologically active variant of human wild-type glucocerebrosidase differs from human wild-type glucocerebrosidase by a single amino acid substitution at K321, or at H145, or at K321 and H145.

Statement 13*. The preparation or composition according to any one of Statements 8* to 12*, wherein the biologically active variant of human wild-type glucocerebrosidase differs from human wild-type glucocerebrosidase by K321N substitution, or by H145L substitution, or by K321N and H145L substitutions.

Statement 14*. The preparation or composition according to any one of Statements 1* to 13*, wherein the mannose of the mannose-6-phosphate moiety is a terminal mannose.

Statement 15*. The preparation or composition according to any one of Statements 1* to 14*, wherein the glycans comprising two mannose-6-phosphate moieties are each independently selected from the group consisting of P₂Man₆GlcNAc₂, and P₂Man₅GlcNAc₂.

Statement 16*. The preparation or composition according to any one of Statements 1* to 14*, wherein the mannose-6-phosphate moiety-comprising glycans are each independently selected from the group comprising or consisting of PMan₇GlcNAc₂, PMan₆GlcNAc₂, PMan₅GlcNAc₂, PMan₄GlcNAc₂, PMan₃GlcNAc₂, P₂Man₆GlcNAc₂, and P₂Man₅GlcNAc₂.

Statement 17*. The preparation or composition according to any one of Statements 1* to 14* wherein the mannose-6-phosphate moiety-comprising glycans are each independently selected from the group comprising or consisting of PMan₅GlcNAc₂, PMan₄GlcNAc₂, PMan₃GlcNAc₂, P₂Man₆GlcNAc₂, and P₂Man₅GlcNAc₂.

Statement 18*. The preparation or composition according to any one of Statements 1* to 14*, wherein the mannose-6-phosphate moiety-comprising glycans are each independently selected from the group comprising or consisting of PMan₃GlcNAc₂ and P₂Man₅GlcNAc₂.

Statement 19*. The preparation or composition according to any one of Statements 1* to 18*, wherein the glucocerebrosidase is obtainable or obtained by uncapping and demannosylation of glucocerebrosidase recombinantly expressed by a fungal cell genetically engineered to produce glucocerebrosidase, in particular genetically engineered to produce glucocerebrosidase comprising glycans at least 10% of which comprise two mannose-1-mannose-6-phosphate moieties.

Statement 20*. The preparation or composition according to any one of Statements 1* to 19*, wherein the glucocerebrosidase is obtainable or obtained by uncapping and demannosylation of glucocerebrosidase recombinantly expressed by a Yarrowia lipolytica genetically engineered to produce glucocerebrosidase, in particular genetically engineered to produce glucocerebrosidase comprising glycans at least 10% of which comprise two mannose-1-phospho-6-mannose moieties.

Statement 21*. A pharmaceutical composition comprising the glucocerebrosidase preparation or composition according to any one of Statements 1* to 20*.

Statement 22*. The pharmaceutical composition according to Statement 21*, wherein the glucocerebrosidase is formulated with artificial cerebrospinal fluid (aCFS).

Statement 23*. The pharmaceutical composition according to any one of Statements 21* or 22*, wherein the pharmaceutical composition has pH of about 6.4 to 6.9, preferably of about 6.6.

Statement 24*. The glucocerebrosidase preparation or composition according to any one of Statements 1* to 20* or the pharmaceutical composition according to any one of Statements 21* to 23*, for use in therapy; or a method for treating a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 25*. The glucocerebrosidase preparation or composition according to any one of Statements 1* to 20* or the pharmaceutical composition according to any one of Statements 21* to 23* for use in a method of treating a disease characterised by glucocerebrosidase deficiency; or a method for treating a disease characterised by glucocerebrosidase deficiency in a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 26*. The glucocerebrosidase preparation or composition according to any one of Statements 1* to 20* or the pharmaceutical composition according to any one of Statements 21* to 23* for use in a method of treating Gaucher disease; or a method for treating Gaucher disease in a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 27*. The glucocerebrosidase preparation or composition according to any one of Statements 1* to 20* or the pharmaceutical composition according to any one of Statements 21* to 23* for use in a method of treating non-neuronopathic Gaucher disease; or a method for treating non-neuronopathic Gaucher disease in a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 28*. The glucocerebrosidase preparation or composition according to any one of Statements 1* to 20* or the pharmaceutical composition according to any one of Statements 21* to 23* for use in a method of treating neuronopathic Gaucher disease; or a method for treating neuronopathic Gaucher disease in a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 29*. The glucocerebrosidase preparation or composition or pharmaceutical composition for use according to Statement 28* or the method according to Statement 28*, wherein the neuronopathic Gaucher disease is type 2 (GD2), type 3 (GD3), or perinatal lethal (GDPL).

Statement 30*. The glucocerebrosidase preparation or composition according to any one of Statements 1* to 20* or the pharmaceutical composition according to any one of Statements 21* to 23* for use in a method of treating glucocerebrosidase-associated alpha-synucleinopathy; or a method for treating glucocerebrosidase-associated alpha-synucleinopathy in a subject in need thereof, the method comprising administering to the subject a prophylactically or therapeutically effective amount of the glucocerebrosidase preparation or composition according to any one of Statements 1* to 20* or the pharmaceutical composition according to any one of Statements 21* to 23*.

Statement 31*. The glucocerebrosidase preparation or composition or pharmaceutical composition for use according to Statement 30* or the method according to Statement 30*, wherein the glucocerebrosidase-associated alpha-synucleinopathy is parkinsonism, Parkinson's disease, Multiple System Atrophy (MSA), or Lewis Body Dementia (LBD).

Statement 32*. The glucocerebrosidase preparation or composition or pharmaceutical composition for use according to any one of Statements 24* to 31*, or the method according to any one of Statements 24* to 31*, wherein the preparation or composition or pharmaceutical composition is administered systemically.

Statement 33*. The glucocerebrosidase preparation or composition or pharmaceutical composition for use according to any one of Statements 24* to 32*, or the method according to any one of Statements 24* to 32*, wherein the preparation or composition or pharmaceutical composition is administered intravenously (IV).

Statement 34*. The glucocerebrosidase preparation or composition or pharmaceutical composition for use according to any one of Statements 24* to 32*, or the method according to any one of Statements 24* to 32*, wherein the preparation or composition or pharmaceutical composition is administered into the central nervous system.

Statement 35*. The glucocerebrosidase preparation or composition or pharmaceutical composition for use according to any one of Statements 24* to 32*, or the method according to any one of Statements 24* to 32*, wherein the preparation or composition or pharmaceutical composition is administered intracerebroventricularly (ICV) or intrathecally.

Statement 36*. The glucocerebrosidase preparation or composition according to any one of Statements 1* to 20* or the pharmaceutical composition according to any one of Statements 21* to 23* for use in a method of treating neuronopathic Gaucher disease or glucocerebrosidase-associated alpha-synucleinopathy by intracerebroventricular (ICV) or intrathecal administration; or a method for treating neuronopathic Gaucher disease or glucocerebrosidase-associated alpha-synucleinopathy in a subject in need thereof, the method comprising intracerebroventricularly (ICV) or intrathecally administering to the subject a prophylactically or therapeutically effective amount of the glucocerebrosidase preparation or composition according to any one of Statements 1* to 20* or the pharmaceutical composition according to any one of Statements 21* to 23*.

Statement 37*. The glucocerebrosidase preparation or composition according to any one of Statements 1* to 20* or the pharmaceutical composition according to any one of Statements 21* to 23* for use in a method of treating neuronopathic Gaucher disease or glucocerebrosidase-associated alpha-synucleinopathy by intracerebroventricular (ICV) administration; or a method for treating neuronopathic Gaucher disease or glucocerebrosidase-associated alpha-synucleinopathy in a subject in need thereof, the method comprising intracerebroventricularly (ICV) administering to the subject a prophylactically or therapeutically effective amount of the glucocerebrosidase preparation or composition according to any one of Statements 1* to 20* or the pharmaceutical composition according to any one of Statements 21* to 23*.

Amino acids with their three letter code and one letter code are listed in Table 1.

TABLE 1
Amino acids with their three letter code and one letter code

| Amino acid | Three letter code | One letter code |
|---|---|---|
| Glycine | Gly | G |
| Alanine | Ala | A |
| Valine | Val | V |
| Leucine | Leu | L |
| Isoleucine | Ile | I |
| Proline | Pro | P |
| Tyrosine | Tyr | Y |
| Tryptophan | Trp | W |
| Phenylalanine | Phe | F |
| Cysteine | Cys | C |
| Methionine | Met | M |
| Serine | Ser | S |
| Threonine | Thr | T |
| Lysine | Lys | K |
| Arginine | Arg | R |
| Histidine | His | H |
| aspartic acid | Asp | D |
| glutamic acid | Glu | E |
| Asparagine | Asn | N |
| Glutamine | Gln | Q |

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1—Structure of Recombinant Glucocerebrosidase Polypeptides

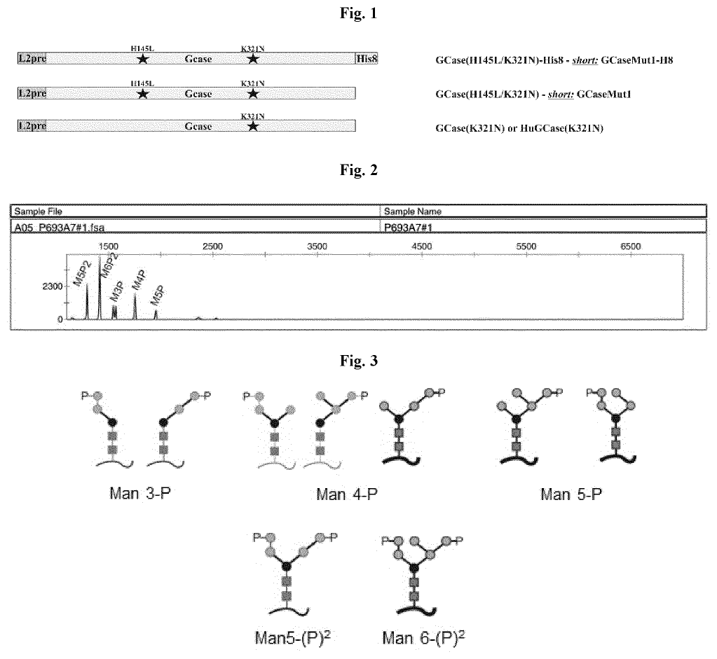

The schematic outline of human glucocerebrosidase (GCase) polypeptides used in preclinical studies reported herein is shown in FIG. 1. “L2pre” denotes the signal peptide from the Yarrowia lipolytica (YL) lipase 2 (Lip2), having the amino acid sequence MKLSTILFTACATLAAA (SEQ ID NO: 6). The two extra Alanine residues (AA) included at the C-terminal end of SEQ ID NO: 6 ensure proper processing of the L2pre in the endoplasmic reticulum. The AA motif is removed by an aminopeptidase. The 2 Alanine residues are essentially the first 2 amino acids of the Lip2 pro-region, immediately following onto the Lip2pre. The L2pre signal peptide is fused to the N-terminus of the respective GCase sequences which lack their native signal peptide, and facilitates secretion of the GCase polypeptides recombinantly produced by YL cells, but is enzymatically removed during processing of the polypeptides within the endoplasmic reticulum, such that the L2pre signal peptide is no longer present in the secreted proteins used for further experiments. “His8” or “H8” denote the poly-histidine tag of eight consecutive histidines (8×His) fused to the C-terminus of the GCase sequence. The position of the single amino acid substitutions H145L and/or K321N is indicated relative to the amino acid sequence of the mature human wild-type GCase, i.e., wherein the native signal peptide has been removed. An example of mature human wild-type GCase is shown in SEQ ID NO: 2 elsewhere in this specification).

The amino acid sequence of the “GCase(H145L/K321N)-His8” polypeptide construct, including the L2pre signal peptide (underlined) that is absent from the mature polypeptide secreted by YL cells, is shown in SEQ ID NO: 7 below; the 8×His tag is in bold:

(SEQ ID NO: 7)
MKLSTILFTACATLAAAARPCIPKSFGYSSVVCVCNATYCDSFDPPTFPA
LGTFSRYESTRSGRRMELSMGPIQANHTGTGLLLTLQPEQKFQKVKGFGG
AMTDAAALNILALSPPAQNLLLKSYFSEEGIGYNIIRVPMASCDFSIRTY
TYADTPDDFQLLNFSLPEEDTKLKIPLIHRALQLAQRPVSLLASPWTSPT
WLKTNGAVNGKGSLKGQPGDIYHQTWARYFVKFLDAYAEHKLQFWAVTAE
NEPSAGLLSGYPFQCLGFTPEHQRDFIARDLGPTLANSTHHNVRLLMLDD
QRLLLPHWAKVVLTDPEAAKYVHGIAVHWYLDFLAPANATLGETHRLFPN
TMLFASEACVGSKFWEQSVRLGSWDRGMQYSHSIITNLLYHVVGWTDWNL
ALNPEGGPNWVRNFVDSPIIVDITKDTFYKQPMFYHLGHFSKFIPEGSQR
VGLVASQKNDLDAVALMHPDGSAVVVVLNRSSKDVPLTIKDPAVGFLETI
SPGYSIHTYLWRRQHHHHHHHH

The amino acid sequence of the “GCase(H145L/K321N)” polypeptide construct, including the L2pre signal peptide (underlined) that is absent from the mature polypeptide secreted by YL cells, is shown in SEQ ID NO: 8 below:

(SEQ ID NO: 8)
MKLSTILFTACATLAAAARPCIPKSFGYSSVVCVCNATYCDSFDPPTFPA
LGTFSRYESTRSGRRMELSMGPIQANHTGTGLLLTLQPEQKFQKVKGFGG
AMTDAAALNILALSPPAQNLLLKSYFSEEGIGYNIIRVPMASCDFSIRTY
TYADTPDDFQLLNFSLPEEDTKLKIPLIHRALQLAQRPVSLLASPWTSPT
WLKTNGAVNGKGSLKGQPGDIYHQTWARYFVKFLDAYAEHKLQFWAVTAE
NEPSAGLLSGYPFQCLGFTPEHQRDFIARDLGPTLANSTHHNVRLLMLDD
QRLLLPHWAKVVLTDPEAAKYVHGIAVHWYLDFLAPANATLGETHRLFPN
TMLFASEACVGSKFWEQSVRLGSWDRGMQYSHSIITNLLYHVVGWTDWNL
ALNPEGGPNWVRNFVDSPIIVDITKDTFYKQPMFYHLGHFSKFIPEGSQR
VGLVASQKNDLDAVALMHPDGSAVVVVLNRSSKDVPLTIKDPAVGFLETI
SPGYSIHTYLWRRQ

The amino acid sequence of the “GCase(K321N)” polypeptide construct, including the L2pre signal peptide (underlined) that is absent from the mature polypeptide secreted by YL cells, is shown in SEQ ID NO: 8 below:

(SEQ ID NO: 8)
MKLSTILFTACATLAAAARPCIPKSFGYSSVVCVCNATYCDSFDPPTFPA
LGTFSRYESTRSGRRMELSMGPIQANHTGTGLLLTLQPEQKFQKVKGFGG
AMTDAAALNILALSPPAQNLLLKSYFSEEGIGYNIIRVPMASCDFSIRTY
TYADTPDDFQLHNFSLPEEDTKLKIPLIHRALQLAQRPVSLLASPWTSPT
WLKTNGAVNGKGSLKGQPGDIYHQTWARYFVKFLDAYAEHKLQFWAVTAE
NEPSAGLLSGYPFQCLGFTPEHQRDFIARDLGPTLANSTHHNVRLLMLDD
QRLLLPHWAKVVLTDPEAAKYVHGIAVHWYLDFLAPANATLGETHRLFPN
TMLFASEACVGSKFWEQSVRLGSWDRGMQYSHSIITNLLYHVVGWTDWNL
ALNPEGGPNWVRNFVDSPIIVDITKDTFYKQPMFYHLGHFSKFIPEGSQR
VGLVASQKNDLDAVALMHPDGSAVVVVLNRSSKDVPLTIKDPAVGFLETI
SPGYSIHTYLWRRQ

For comparative experiments, imiglucerase (INN) for injection (CAS #154248-97-2), commercially available under the brand name Cerezyme® from Genzyme Europe B.V., Naarden, the Netherlands, was used. Imiglucerase is a recombinant human glucocerebrosidase produced in mammalian Chinese Hamster Ovary (CHO) cell culture. Imiglucerase is a monomeric glycoprotein of 497 amino acids containing 4 N-linked glycosylation sites, and differs from placental glucocerebrosidase by one amino acid at position 495 where arginine is substituted by a histidine. The oligosaccharide chains at the glycosylation sites have been modified to terminate in mannose sugars, which are recognised by endocytic carbohydrate receptors on macrophages.

For certain comparative experiments, also velaglucerase alpha (INN) for injection, commercially available under the brand name VPRIV® from Shire Pharmaceuticals Ireland Limited, was used. Velaglucerase alpha has the same amino acid sequence as wild-type human glucocerebrosidase and is recombinantly produced in HT-1080 human fibroblast cell line.

### Example 2—Production of Fungal Cells Expressing the Recombinant Glucocerebrosidase (GCase) Polypeptides

Nucleic acids encoding the glucocerebrosidase K321N or H145L/K321N GCase variants as described in Example 1 were synthesised with codon optimisation for expression by Yarrowia lipolytica, and addition of a 8×His tag where indicated. The obtained coding sequences were cloned in frame after the L2pre signal peptide. The nucleotide sequence of the codon optimised open reading frame (ORF) encoding the GCase(H145L/K321N)-His8 polypeptide is shown in SEQ ID NO: 9 below, with the sequences encoding the L2pre signal peptide and the 8×His tag underlined and bold, respectively. The start codon and stop codon are italicised. The codons for L145 and N321 are framed.

(SEQ ID NO: 9)
atgaagctgtccaccattctcttcaccgcctgtgctaccctcgccgccgctgctcgaccatgcatccccaagtccttcggctactcctctgtcgtgt
gtgtctgcaacgctacctactgtgactctttcgacccgcccaccttccccgctctgggcaccttctcccgatacgagtctacccgatctggacgac
gaatggagctctctatgggtcccattcaggctaaccacaccggtaccggactgctcctcaccctgcagcccgagcagaagttccagaaggtga
agggtttcggtggagctatgaccgacgctgctgccctcaacatcctggctctctctcccccggctcagaacctcctgctgaagtcctacttctctg
aggaaggtattggctacaacatcattcgagtgcccatggcctcctgcgacttctctatccgaacctacacctacgccgacacccccgacgacttc

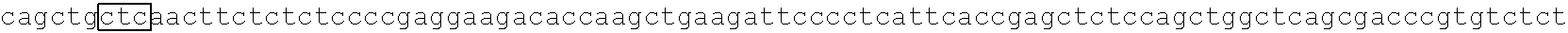

cctggcttctccctggacctctcccacctggctcaagaccaacggtgccgtcaacggcaagggatctctgaagggccagcccggagacatcta
ccaccagacctgggctcgatacttcgtgaagttcctcgacgcctacgctgagcacaagctgcagttctgggctgtcaccgccgagaacgagcc
ctctgccggactgctctccggttaccccttccagtgtctcggtttcacccccgagcaccagcgagacttcattgcccgagacctcggtcccaccc
tcgccaactccacccaccacaacgtccgactgctgatgctcgacgaccagcgactcctcctgccccactgggccaaggtggtcctgaccgac

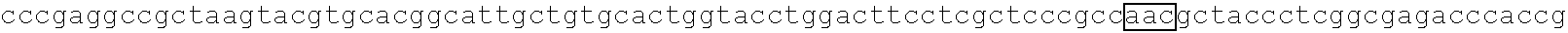

actgttccccaacaccatgctgttcgcctctgaggcttgcgtgggttccaagttctgggagcagtccgtgcgactgggttcctgggaccgagga
atgcagtactctcactctattatcaccaacctgctgtaccacgtcgtgggttggaccgactggaacctcgctctcaaccccgagggtggacccaa
ctgggtccgaaacttcgtcgactctcccattatcgtcgacatcaccaaggacaccttctacaagcagcccatgttctaccacctgggacacttctct
aagttcattcccgagggctcccagcgagtgggactggtggcttctcagaagaacgacctcgacgctgtcgccctgatgcaccccgacggctct
gccgtcgtggtcgtcctcaaccgatcctctaaggacgtccccctcaccattaaggaccccgctgtcggtttcctggagaccatctctcccggttac
tctatccacacctacctctggcgacgacagcaccaccaccaccaccaccaccactaa

The GCase(H145L/K321N) ORF is substantially identical to SEQ ID NO: 9, but lacking the nucleotides in bold coding for the 8×His tag. The GCase(K321N) ORF is substantially identical to SEQ ID NO: 9, but lacking the nucleotides in bold coding for the 8×His tag, and having the histidine-encoding codon CAC instead of the L145 codon CTC.

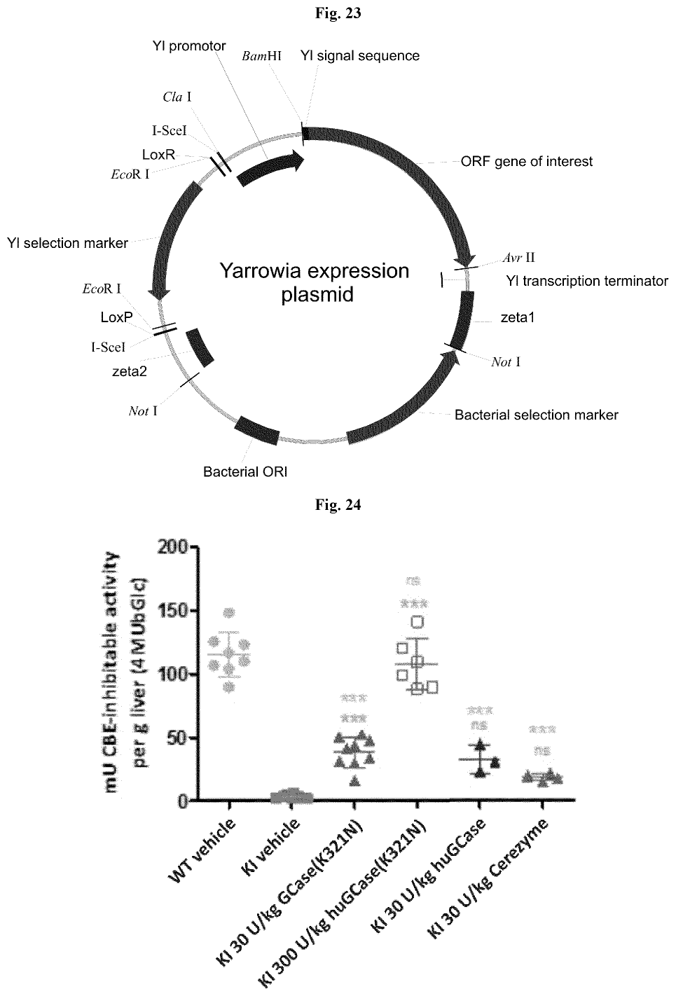

Each GCase ORF was introduced into an YL expression vector (schematically represented in FIG. 23) under the control of Hp4d promoter. Following propagation and isolation of the vector from E. coli, the vector was digested by Not I restriction nuclease to remove the bacterial sequences, and obtain an integrative fragment containing the GCase expression cassette and a YL selection marker. The integrative fragments were separated by agarose gel electrophoresis followed by Qiagen column purification. Transformation of YL cells with the respective integrative fragments and selection of transformants was carried out according to well established protocols.

The respective GCase ORFs were transformed into YL cells, genetically engineered to synthesize high amounts of phosphorylated N-glycans onto secreted glycoproteins. This glyco-engineered strain is derived from the laboratory strain po1d (CLIB139, available from Collection de Levures d'Intérêt Biotechnologique, CIRM-Levures, Research Center INRA, Domaine de Vilvert, Bat. 442, 78352 Jouy-en-Josas, France, https://www6.inra.fr/cirm_eng/Yeasts/Strain-catalogue), a derivative of wild type strain W29 (ATCC® 20460™, available from American Type Culture Collection, 10801 University Blvd. Manassas, Va. 20110-2209, USA, www.atcc.org), and has the following genotype: Mat A, ura3-302, leu2-270, ade2-844, xpr2-322. The strain comprises further genetic modifications including in particular:
Deletion of the OCHI gene: This abrogates the potential of synthesizing hyperglycosyl structures onto secreted glycoproteins. The main N-glycan on total extracellular protein is neutral Man₈GlcNAc₂.
Targeted integration of two Hp4d promotor-driven expression cassettes of the Yarrowia lipolytica MNN4 gene: This results in the conversion of almost all neutral N-glycans into structures containing one or two phosphomannose moieties. The main N-glycan on total extracellular protein are ManP-Man₈GlcNAc₂ and (ManP)₂-Man₈GlcNAc₂.

The transformed YL cells were grown in controlled bioreactor cultivations to overexpress the respective GCase ORFs, which resulted into their secretion within the fermentation broth. The standard fermentation process consisted of 3 main cultivation phases: pre-cultivation from a single colony, pre-culture cultivation to produce biomass as the starting material for the main fermentation, and main fermentation including a batch phase and one or more feed phases. Standard YSG (1% w/v yeast extract; 2% w/v soyton; 2% v/v glycerol) medium was used for pre-cultivation and pre-culture cultivation, while defined medium using glycerol as carbon source (5 g/L) was used in main fermentation. In the one or more feed phases, 600 g/L glycerol, 4.6% soyton, and trace elements were added.

### Example 3—Isolation, Uncapping and Demannosylation of the Recombinant Glucocerebrosidase (GCase) Polypeptides

The purification process for His-tagged GCase variants was based on Ni-IMAC chromatography steps. Clarified fermentation broth was loaded onto a first Ni-IMAC column (Chelating Sepharose FF). After washing with 50 mM imidazole, the His-tagged GCase was eluted with 400 mM imidazole. A buffer exchange towards 50 mM sodium citrate buffer pH 4.5 was performed on the eluted fraction. The ZnCl₂ concentration was adjusted to 0.2 mM and Jack Bean alpha-mannosidase was added in a 15/100 mannosidase/GCase weight for weight ratio. The mixture was incubated for 16 hours at 30° C. and shaking at 90 rpm in order to allow the mannosidase to remove the phosphate-capping mannose residues and to further demannosylate the protein-linked N-glycans.

After incubation, the product was centrifuged for 10 min (at 4° C., 4000 g) to remove precipitated material. The supernatant was buffer exchanged into 50 mM sodium phosphate buffer 100 mM NaCl pH 6.2 and loaded for a second time on a Ni-IMAC column (Chelating Sepharose FF) to remove the Jack Bean mannosidase and residual host cell proteins. For this purpose, the column was washed with 100 mM and His-tagged GCase was eluted with 400 mM imidazole. The eluted fraction was buffer exchanged to 50 mM sodium citrate buffer pH 6.0, and ammonium sulphate was added to a final concentration of 0,9 M. The mixture was loaded on a hydrophobic interaction column (Ether 650-M) as a final polishing step. His-tagged GCase was eluted in 50 mM sodium citrate buffer pH 6.0 with a purity of >98%.

One protocol used for purification of untagged GCase variants is described below. Upon addition of ammonium sulphate (0.9 M final concentration) to the harvested and clarified fermentation broth, a hydrophobic interaction chromatography (HIC) on a PPG-600M resin was used as a capturing step for the secreted GCase variants. The protein of interest was eluted from the PPG column by applying a 10 mM sodium phosphate buffer, pH 6.2. The HIC elution fraction was exchanged to 20 mM sodium citrate pH 6.0 and further adjusted to pH 4.5 by spiking of 250 mM sodium citrate buffer pH 4.0. As an intermediate purification step, the material was then processed via cation exchange chromatography (CEC) on a Fractogel EMD SE resin. The GCase was eluted from the column by applying a NaCl gradient from 0 to 1000 mM. Fractions containing the GCase were pooled. The ZnCl₂ concentration of the pool was adjusted to 0.2 mM and Jack Bean alpha-mannosidase was added in a 15/100 mannosidase/GCase weight for weight ratio. The mixture was incubated for 16 hours at 30° C. and shaking at 90 rpm in order to allow the mannosidase to remove the phosphate-capping mannose residues and to further demannosylate the protein-linked N-glycans. After incubation, the product was centrifuged for 10 min (at 4° C., 4000 g) to remove precipitated material. After exchanging the supernatant into 20 mM sodium phosphate buffer pH 4.5, a second cation exchange chromatography step (Fractogel EMD SE resin) served to remove the added Jack Bean alpha-mannosidase and to further reduce the host cell protein content. Proteins were eluted from the column by applying a 0 to 1000 mM NaCl gradient. Fraction containing the GCase were pooled, followed by a buffer exchange to 50 mM sodium citrate, pH 6.0 and the addition of ammonium sulphate up to a final concentration of 0.9 M. The product was then loaded on a second hydrophobic interaction column (Ether-650 M), which served as a final polishing chromatography step. A gradient from 0.9 M to 0 M ammonium sulphate was applied to elute bound proteins and all fractions containing only the full-size GCase product were pooled. The introduction of this second HIC step resulted into a final GCase purity of >98%.

In the present Examples, the GCase(H145L/K321N)-His8, GCase(H145L/K321N), or GCase(K321N) glucocerebrosidase variants, particularly their uncapped and demannosylated form, may each be referred to by the label “OxyGCase”. The Examples and/or context define which variant is meant in which situation.

### Example 4—N-Glycan Structures of the Recombinant Glucocerebrosidase (GCase) Polypeptides

N-glycans were released in solution (3 hours at 37° C.) from up to 10 μg of denatured uncapped and demannosylated GCase polypeptides with N-Glycosidase F (PNGaseF). Upon incubation, 4 volumes of ice-cold acetone were added and the mixture was incubated for at least 20 minutes at −20° C. After centrifugation for 5 minutes at 13.000 rpm, the supernatant was removed. To the pellet, containing a mixture of precipitated proteins and released N-glycans, 60% ice-cold methanol was added to solubilize the N-glycans. After a centrifugation step (5 minutes, 13.000 rpm), the supernatant containing the N-glycans was collected and dried at 60° C. in a vacuum centrifuge. Dried N-glycan samples were labelled with APTS (8-amino-1,3,6-pyrenetrisulfonic acid trisodium salt) and, upon removal of excess unreacted label, subsequently analysed on DSA-FACE (DNA Sequencer-Aided Fluorophore-Assisted Carbohydrate Electrophoresis). The method of glycan labelling, clean-up and electrophoresis essentially follows the protocol described in Laroy et al. Nature Protocols 2006, vol. 1, 397-405.

A representative DSA-FACE electropherogram of the isolated N-glycans of one of the uncapped and demannosylated GCase polypeptides as prepared herein, including peak annotation, is shown in FIG. 2. Similar profiles were obtained for all GCase polypeptides as prepared herein (not shown). Substantially all detectable N-glycans were phosphorylated, with a very high proportion being bi-phosphorylated (M5P2, M6P2). The N-glycan structures corresponding to the annotations in FIG. 2 are depicted in FIG. 3.

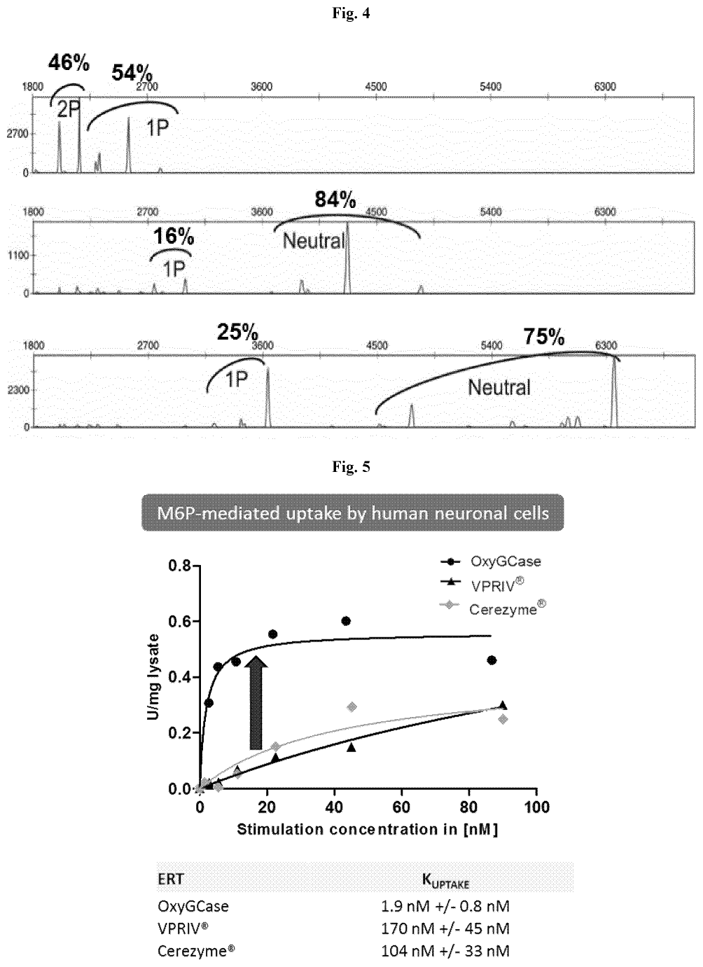

The N-glycan structures were similarly determined for the Cerezyme® and VPRIV® preparations. FIG. 4 shows a representative DSA-FACE electropherogram of the isolated N-glycans of one of the uncapped and demannosylated OxyGCase polypeptides (top panel), Cerezyme® (middle panel), and VPRIV® (bottom panel), including annotation of peaks corresponding to bi-phosphorylated (2P), monophosphorylated (IP) and non-phosphorylated (Neutral)N-glycans. In the top panel, representing an embodiment of the presently described GCase, substantially all detectable N-glycans were phosphorylated, with 46% (by number) bi-phosphorylated N-glycans and 54% (by number) monophosphorylated N-glycans. In the middle panel, representing Cerezyme®, only 16% (by number)N-glycans were phosphorylated, more particularly monophosphorylated, with the rest being neutral. Bi-phosphorylated N-glycans were substantially not detectable. In the bottom panel, representing VPRIV®, only 25% (by number) of N-glycans were phosphorylated, more particularly monophosphorylated, with the rest being neutral. Bi-phosphorylated N-glycans were substantially not detectable.

### Example 5—Uptake of the Recombinant Human Glucocerebrosidase (GCase) Polypeptides by Neuronal Cells and Microglia

Cultured human neuroblastoma cells (SH-SY5Y—ATCC® accession number CRL-2266) were contacted with the uncapped and demannosylated OxyGCase polypeptide, and GCase uptake was measured. Cerezyme® and VPRIV® were used as controls. Essentially, cells were seeded in growth medium at 0.8×10⁵ cells per individual well of a 24-well plate. Since the SH-SY5Y cells have endogenous glucocerebrosidase activity, they were first treated overnight with the irreversible inhibitor conduritol B epoxide (CBE) before stimulation with different concentrations (done in duplicate) of exogenously added glucocerebrosidase variants. After two hours, the stimulated cells were lysed and enzyme uptake (expressed as units per mg total protein) was determined on the lysate using the 4-Methylumbelliferyl-β-D-glucopyranoside (4MUβGlc) assay. This assay is based on the fact that GCase is able to convert the fluorogenic 4MUβGlc into glucose and 4-methylumbelliferone (4-MU) under acidic conditions (pH 4.5) and compatible temperature (37° C.). After a defined amount of time, the reaction was stopped by adding an alkaline stop solution, which in turn also maximizes the fluorescence intensity of the released 4-MU. Fluorescence emission was measured at 460/40 nm upon excitation at 360/40 nm. Under the currently used assay conditions, the intensity of the fluorescent signal is proportional to the amount of active enzyme and can be converted to the amount of released 4-MU (expressed in μmol) based on the fluorescence values of a 4-MU standard curve. One unit of activity is considered as the amount of enzyme that catalyses the hydrolysis of 1 μmol of 4MUβGlc (or the release of 1 μmol of 4-MU) per minute, at 37° C. and at a substrate starting concentration of 5 mM within the following assay buffer: 111 mM Na₂HPO₄, 44 mM citric acid, 0.5% BSA, 10 mM sodium taurocholate, 0.25% Triton-X-100, pH 5.5. The specific activity (units/mg) of the enzyme preparation was determined by dividing the measured units/mL by the established protein concentration (expressed in mg/mL, e.g. determined via OD280 measurement).

Unstimulated CBE-treated cells were used to determine background activity levels within the lysates. The normalized glucocerebrosidase activities per mg lysate proteins were plotted against the stimulation concentrations (in nM) in Graphpad Prism. The generated data points were fit using a hyperbolic curve, describing the relationship between rate of uptake and applied enzyme concentration during stimulation to allow determination of the K_{update} values for the tested enzyme variants. To further demonstrate the mechanism of uptake, cells were also stimulated in the presence of either M6P, mannan or both to specifically block uptake via resp. the M6P receptor (M6PR), the mannose receptor or both. The difference in degree of cell-uptake is best exemplified when plotting the curves for the net M6PR mediated uptake (i.e. after subtracting the values for the non-M6PR mediated cell-uptake from the values of the overall cell-uptake) for all three enzymes onto the same graph (FIG. 5), showing that OxyGCase was taken up by neuronal cells to a much greater extent (K_{UPTAKE}=1.9+/−0.8 nM) than either Cerezyme® (K_{UPTAKE}=104+/−33 nM) or VPRIV® (K_{UPTAKE}=170+/−45 nM).

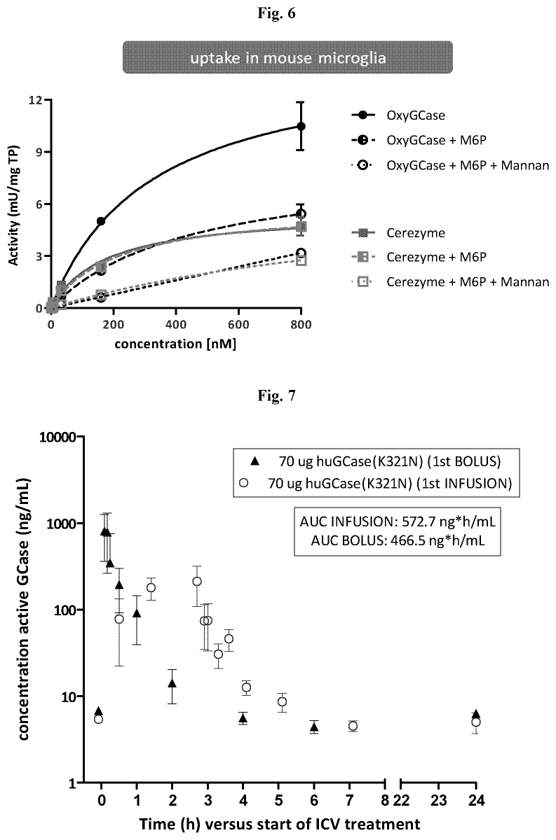

Cultured mouse microglia (ATCC® accession number CRL-2467) were contacted with OxyGCase as described above, and GCase uptake was measured. Cerezyme® was used as control. In certain experiments, mannose-6-phosphate (M6P) was added to compete with M6P receptors on the cells. In certain experiments, both M6P and mannan were added to compete with both M6P and mannose receptors on the cells. FIG. 6 shows that OxyGCase was taken up by mouse microglia more efficiently than Cerezyme®. Addition of M6P reduced OxyGCase uptake, such as to be similar to the uptake of Cerezyme® alone, consistent with competition with the M6P receptor-mediated fraction of the uptake. Addition of M6P+mannan reduced OxyGCase uptake even further. M6P did not observably reduce the uptake of Cerezyme®, consistent with the fact that Cerezyme® substantially lacks phosphorylated mannose-containing N-glycans. M6P+mannan reduced the uptake of Cerezyme®, such as to be substantially the same as the uptake of OxyGCase+M6P+mannan.

### Example 6—Mouse Model for Neuronopathic Gaucher Disease

The present Examples employ the Gaucher model Gba1 D409V knock-in (KI) mouse. The Gba1 D409V KI mouse was generated as a model for type 3 Gaucher disease and Parkinson's disease (Dave et al. https://www.michaeljfox.org/files/foundation/MJFFGBA_SFN_OCT2015.pdf), and is available at The Jackson Laboratory Stock #019106. These mice express the mutant D427V mouse Gba1 protein, which corresponds to one of the most prevalent human GBA1 mutations in Gaucher patients (D409V) (Hruska. Gaucher disease: mutation and polymorphism spectrum in the glucocerebrosidase gene (GBA). Hum Mutat. 2008, vol. 29, 567-83). The Gba1 D409V KI mice advantageously display longer lifespan in comparison to the severe type 2 Gaucher mouse models (K14-Cre gba^{lnl/lnl} and Nestin-Cre gba^{flox/flox}), having a lifespan of only 2-3 weeks. Homozygous Gba1 D409V KI mice had been previously shown to accumulate one of the GCase substrates, glycosylsphingosine (GlcSph), in both brain and liver (Dave et al. supra).

### Example 7—Intracerebroventricular (ICV) Delivery of the Recombinant Human Glucocerebrosidase (GCase) Polypeptides in Mice

For studies described in ensuing Examples 7-12, 18- to 27-week old mice were implanted with a unilateral cannula. To confirm the appropriate site of cannula implantation, cerebrospinal fluid (CSF) was pulled from the lateral ventricle at the start of the first infusion and mice were infused with methylene blue immediately before sacrifice. Mice were treated weekly (EW), bi-weekly (BW) or every other day (EOD) with a bolus (10-20 min) or a slow infusion (3 h) of test article for 1-12 consecutive weeks. In some of the studies, plasma was collected at different time points after ICV treatment. Three hours, 48 hours or 1 to 2 weeks after the last infusion, mice were anaesthetized and blood and CSF (in the final study) were collected, followed by saline perfusion and dissection of brain and liver. Tissue samples were homogenized for further analysis as described below.

Hexosylsphingosine (HexSph) levels (comprising the 2 epimers, GlcSph and GalSph) were analysed via RP-LC Q-TOF-MS (Reverse Phase-Liquid Chromatography coupled to high-resolution Quadrupole Time-of-Flight Mass Spectrometry) analysis. In a subgroup of animals, the differentiation between GlcSph and GalSph was made via SPE-HILIC-MS (Solid Phase Extraction-Hydrophilic Interaction Liquid Chromatography-Mass Spectrometry) analysis. The homogenization buffer for HexSph analyses consisted of methanol spiked with the internal standards GlcSph-d5 (#860636P, Avanti Polar Lipids; stock 1 ppm or 1 ng/mL) and C18 GlcCer-d5 (#860638P, Avanti Polar Lipids; stock 20 ppm or 20 ng/mL) at a final concentration of 5 ng GlcSph-d5 and 100 ng C18 GlcCer(d18:1-d5/18:0) per 300 μL methanol. The homogenization buffer for GalSph and GlcSph analyses consisted of acetone spiked with the internal standards GlcSph-d5 (#860636P, Avanti Polar Lipids; stock 1 ppm or 1 ng/mL) and C18 GlcCer-d5 (#860638P, Avanti Polar Lipids; stock 200 ppm or 200 ng/mL) at a final concentration of 40 ng GlcSph-d5 and 400 ng C18 GlcCer(d18:1-d5/18:0) per mL acetone. For both analyses, the tissue was homogenized at a concentration of 200 mg/mL with the Precellys® Mini bead homogenizer (Bertin) using Precellys® tubes (# KT03961-1203.0.5) and 1.4 mm zirconium oxide beads (#KT03961-1-103.BK) for 2 times 30 sec at 5000 rpm with a 15-sec interval. After centrifugation for 15 min at 14000 rpm, the supernatant was transferred to a new tube of which 300 μL was used for HexSph (and HexCer) analysis and 250 μL for GlcSph and GalSph analysis.

For the HexSph analysis, lipid extraction was performed by adding 1 mL methyl-tert-butylether (MTBE) to 300 μL of the homogenate supernatant. After shaking (1 hour, room temperature), 260 μL water was added followed by another shaking and incubation step (10 minutes, room temperature). After centrifugation (10 minutes, 1000 g), the upper phase was collected, vacuum evaporated and reconstituted in 2/1 methanol/chloroform (v/v). This lipid fraction was then further analyzed via RP-LC Q-TOF-MS. The LC-MS method was adapted from Sandra et al. (Journal of Chromatography A. 2010, vol. 1217, 4087-4099. The following analytical conditions were applied: Column: Acquity UPLC BEH Shield RP18 column (2.1×100 mm; 1.7 μm; Waters, Milford, Mass., USA)—column temperature of 80° C.—injection volume of 10 μL; Mobile phases: A=20 mM ammonium formate pH 5; B=methanol; Flow rate: 0.5 mL/min; Gradient: 0-5 min at 50-74% B; 5-6 min at 74-85% B; 6-16 min at 85-90% B; 16-17 min at 90-94% B; 17-26 min at 94-100% B and Post-time of 9 min at 50% B.

High-resolution accurate mass spectra were obtained with an Agilent 6545 Q-TOF mass spectrometer (MS) (Agilent Technologies) equipped with a dual Jetstream electrospray ionization (ESI) source. The instrument was operated in positive electrospray ionization mode. Chromatographic separation was achieved on an Agilent 1290 Infinity II LC system (1290 High Speed Pump, G7120A; 1290 Multisampler, G7167B; 1290 MCT, G7116B; Agilent Technologies). A stand-alone Sandra/Selerity Series 9000 Polaratherm oven (Selerity Technologies, Salt Lake City, Utah, USA) was used for temperature control of the analytical column. Raw data were processed using the accompanying MassHunter Qualitative Analysis software package (B.07 SP1, Agilent Technologies).

For the GlcSph+GalSph analysis, lipid extraction on the supernatant was performed by adding 1.5 mL acetone to the 250 μL homogenate, followed by intensive vortexing and a centrifugation step (10 minutes at 15.000 g) after which the supernatant was dried by centrifugal vacuum evaporation and reconstituted in 500 μL 2/1 chloroform/methanol (v/v). On these samples, a solid phase extraction (SPE) is performed by loading the samples on SPE cartridges (Sep-Pak Vac 1 cc Accell Plus CM (Waters, # WAT023625), conditioned with 2×1 mL chloroform/methanol 2/1 (v/v), followed by collecting the flow through (=breakthrough fraction). The elution was performed by eluting 4 times with 500 μL of chloroform/methanol/water 30/60/8 (v/v/v). The first fraction was collected together with the breakthrough fraction and consists of Glc- and GalCer. Glc- and GalSph are collected in the second to fourth elution fraction. The fractions were dried by centrifugal vacuum evaporation. Both fractions were dissolved in 20 μL methanol and separated on a HILIC column (Zorbax HILIC Plus RR HD (2.1×150 mm, 1.8 μm)) with an isocratic elution consisting of acetonitrile/water/methanol 86/7/7 v/v/v+0.1% formic acid+315 mg/L ammonium formate. The applied flow rate is 0.8 mL/minute and the column temperature is 25° C.

Analysis of the GCase levels within the tissues was performed via the 4MUβGlc activity assay (essentially as described in Example 5) or by alphaLISA. Homogenisation of tissue samples (to 1 weight volume of brain tissue 5 weight volumes of homogenization buffer are added (giving 200 mg tissue/mL)) was performed using the Precellys® Mini bead homogenizer (Bertin Technologies) and Precellys® tubes pre-filled with 1.4 mm zirconium oxide beads (0.5 mL tubes, ref # P000933-LYSKO-A or 2 mL tubes, VWR ref #432-3751). The homogenization buffer used for compatibility with the activity assay and alphaLISA consisted of 111 mM Na₂HPO₄, 44 mM citric acid, 10 mM sodium taurocholate, 0.25% Triton X-100 and protease inhibitor cocktail (cOmplete™-EDTA-free, Roche, #04693159001), adjusted to pH 5.5. Tissue disruption with the beads occurs for 2 times 30 sec at 5000 rpm with a 15-sec interval. After centrifugation for 15 min at 10.000 g, the supernatant was aliquoted and stored at −80° C. or further analyzed. The AlphaLISA bead-based technology relies on PerkinElmer's exclusive amplified luminescent proximity homogeneous assay (AlphaScreen®) and uses a luminescent oxygen-channeling chemistry. The developed GCase AlphaLISA assay was based on the capturing of huGCase by a biotinylated anti-huGCase antibody bound to streptavidin-coated donor beads (Perkin Elmer, #6760002B) and a second anti-huGCase antibody conjugated to AlphaLISA acceptor beads (Perkin Elmer, #331383). Antibody biotinylation was performed to a concentration of 0.6 mg/mL in PBS pH 7.4; biotinylated antibodies were used at a concentration of 0.00625 μM in 1× HiBlock buffer (prepared from 10× HiBlock buffer, Perkin Elmer, # AL004F). Antibody conjugation towards acceptor beads was performed to a concentration of 5 mg/mL in PBS+0.05% Proclin 300; for use in the alphaLISA assay, the conjugated antibodies were diluted to 100 μg/mL in 1× HiBlock buffer. Just before use, the AlphaScreen Streptavidin Donor Beads were diluted to 150 μg/mL in 1× HiBlock buffer. The binding of the two antibodies to GCase brings donor and acceptor beads into proximity. Laser irradiation of donor beads at 680 nm generates a flow of singlet oxygen, triggering a cascade of chemical events in nearby acceptor beads, which results in a chemiluminescent emission at 615 nm. The emission signal is proportional to the huGCase concentration in the well. A huGCase standard curve was used to calculate the GCase concentration in the tissue samples.

### Example 8—Plasma Pharmacokinetics of the Recombinant Human Glucocerebrosidase (GCase) Polypeptides in Mice

The kinetics of active OxyGCase in blood was determined via an enzyme activity assay using 4MUβGlc as substrate. Gba1 D409V KI mice were intracerebroventricularly (ICV) infused with 70 μg of huGCase(K321N) via a bolus injection (˜15 min, n=11) or a slow infusion (˜3 h, n=6). Blood collected at different time points during and after ICV infusion was immediately buffered with 130 mM citrate buffer pH 5.8 (1:1) in order to prevent GCase activity loss at higher pH, before plasma preparation. Plasma was prepared for 4MUβGlc activity measurement, essentially as described in Example 5. The resulting pharmacokinetics (PK) curves are shown in FIG. 7 and FIG. 8.

The maximum concentration in circulation after injection of huGCase(K321N) in the lateral ventricle was lower for a slow infusion compared to a bolus infusion. However, the total drug exposure was similar for both infusion rates (particularly after a first infusion) as indicated by the similar AUC. In both cases, GCase was cleared fast from circulation. As shown in Examples 9 and 10, a large amount of circulating GCase ended up in the liver.

The PK parameters did not significantly alter after the first or the fourth slow infusion despite significant animal variation, as can be observed in FIG. 8 (right panel). In contrast, the AUC doubled due to a slower clearance from circulation upon multiple bolus ICV injections (FIG. 8, left panel).

### Example 9—Biodistribution of the Recombinant Human Glucocerebrosidase (GCase) Polypeptides in Mice

Biodistribution (BD) of OxyGCase was assessed via several orthogonal assays over various studies. These assays served the purpose of determining the extent of OxyGCase diffusion throughout the brain (contralateral side of de cannula implantation, deeper brain regions, CSF, etc.) and throughout peripheral organs such as the liver.

BD Assessed Through ABP-Labelled GCase

Witte et al. (Ultrasensitive in situ visualization of active glucocerebrosidase molecules. Nat Chem Biol. 2010, vol. 6, 907-913) developed a technology to visualize GCase molecules employing activity-based probes (ABPs).

Fluorescent boron-dipyrromethene-containing cyclophellitol β-epoxide is hijacking the catalytic double-displacement mechanism of GCase to form an irreversible inhibitor-nucleophile adduct (FIG. 9, right panel). This covalent labelling is highly specific and the detection of fluorescent labelled enzyme is ultra-sensitive (detection limit in the attomol range). The red MDW941 β-epoxide ABP (FIG. 9, left panel) is used to label OxyGCase, essentially as described in Kallemijn et al. (A sensitive gel-based method combining distinct cyclophellitol-based probes for the identification of acid/base residues in human retaining β-glucosidases. J Biol Chem. 2014, vol. 289, 35351-62).

Wild-type (WT) mice were unilaterally ICV infused with 10 μg ABP-labelled GCaseMut1-H8 (see FIG. 1) at an infusion rate of either 0.1 μL/min for 20 minutes or 1 μL/min for 2 minutes. Blood, CSF, brain and liver tissue were collected 1 hour or 3 hours after infusion. Biodistribution was determined by quantifying the amount of ABP label. For this, frozen brain tissue was homogenized in 25 mM potassium phosphate buffer, pH 6.5, supplemented with 0.1% (v/v) Triton X-100 and protease inhibitor at a tissue:volume ratio of 1:10 (50 mg tissue in 500 μl buffer), using a Kimble Kontes drive unit with a glass pestle and tube at 2000-3000 rpm. Homogenates were then centrifuged at 10.000 g (at 4° C.), aliquoted and stored in the dark. An aliquot was used to determine total protein concentration via the Bradford assay. To another aliquot, Laemmli buffer was added and the sample was boiled for 4 min at 96° C. before loading on a 4-15% (w/v) SDS-PAGE gel. During gel electrophoresis, the apparatus was covered to avoid exposure to light. The following amounts were loaded for analysis: brain homogenate: 24 μl; serum: 5 μl and CSF: 0.5 μl. 5 calibration samples of labelled GCase (including 0 as well as a range from 8, 40, 200, till 1000 femtomole) spiked in 100% total brain homogenate (pooled from 6 regions of one control animal), serum (of the pool of three control animals) or CSF (from one control animal) were loaded to generate a calibration curve. Calibration samples contained the same amount of tissue as the experimental samples (24, 5 and 0.5 μl for the respective tissues). Wet slab gels were scanned for fluorescence using the FLA-5000 imaging system (Fujifilm life science) at excitation 532 nm and emission wavelength 610 nm. Gel images were visualized in ImageJ, and for every lane the band corresponding to GCase, as well as the space above and under this band, were manually selected. After plotting relative densities using the ‘Plot lanes’ function, the peak above background level was selected and quantified. The slope and intercept of a linear trend line and the detection limit were calculated using the densities of the 40, 200 and 1000 fmol bands of each gel. Using the slope and intercept, the band densities of the experimental samples were converted to fmol loaded per lane. For brain samples, the quantity per lane was corrected for protein concentration. Then the total amount of labelled GCase per brain area was calculated using the homogenization volume. The amount of GCase per mg tissue was calculated using the tissue weight. For serum and CSF samples, the total amount of labelled GCase per 1d was calculated using the tissue volume loaded on gel (5 and 0.5 μl, respectively). Total GCase detected per animal (brain, serum and CSF) was calculated assuming a CSF volume of 35 μl and a serum volume of 1500 μl.

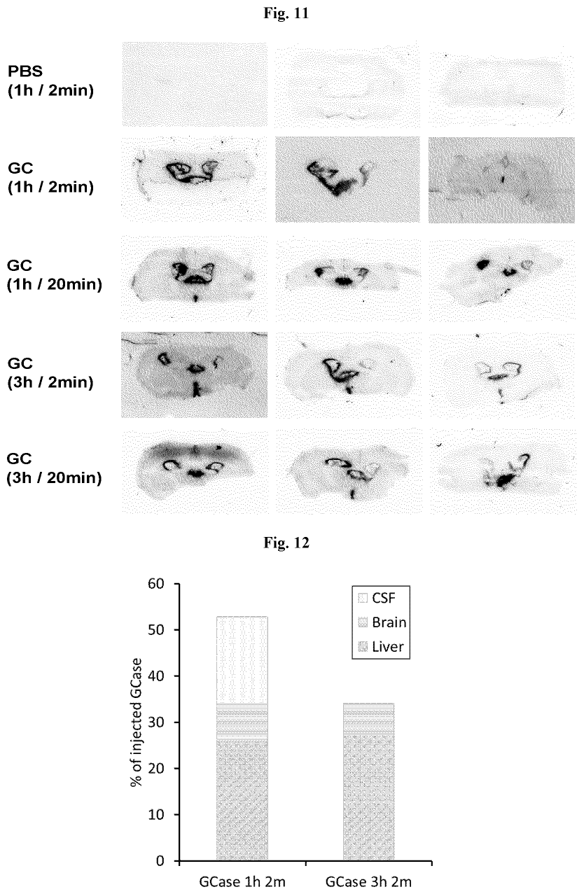

As shown in FIG. 10, the ABP label can be detected throughout the brain, including the contralateral injection side, with the highest concentrations in the posterior areas (5 and 6). There was no apparent difference in distribution between 0.1 μL/min (20 m) and 1 μl/min (2 m) infusion. Labelled GCaseMut1-H8 was detected in similar amounts in brain homogenates 1 hour and 3 hours after infusion: approximately 12-14 μmol or 7-8% of the total infused dose of GCaseMut1-H8 (167 μmol). A significant portion of the GCaseMut1-H8 in the brain was located around the lateral ventricles (FIG. 11). However, after analyzing brain regions devoid of ventricles collected 3 hours after ICV infusion, it was evident that ABP-labelled GCaseMut1-H8 also distributed to deeper brain areas, albeit to a lesser extent (not shown). Based on a rough estimation, 3.7 μmol or 0.8% of the injected dose (460 μmol) was present in deeper brain regions 3 hours after infusion.

1 hour after infusion, approximately 20% of the injected dose was detected in cerebrospinal fluid (CSF). However, ABP-labelled GCaseMut1-H8 could no longer be detected 3 hours after infusion in CSF. Approximately every 2 hours, the complete volume of CSF is replenished (Stroobants et al. Intracerebroventricular enzyme infusion corrects central nervous system pathology and dysfunction in a mouse model of metachromatic leukodystrophy. Hum Mol Genet. 2011, vol. 20, 2760-9), suggesting that 1 hour after infusion there was significant distribution of GCaseMut1-H8 throughout the ventricular system, and that 3 h after infusion the GCaseMut1-H8 was absorbed from the CSF (through the ventricle walls or CSF drainage routes).

Analysis of blood samples confirmed the fast clearance to and from the circulation as described above. Quantification of the ABP label in serum was hampered by high background in this matrix. The ABP label was additionally quantified in liver tissue (preparation of homogenates and analysis of samples was essentially the same as for brain tissue) and results indicate that a significant amount of ABP-labeled GCaseMut1-H8 (˜25%) that was injected into the CSF ended up in the liver as fast as 1 hour after infusion. This appears to be the maximum since similar levels of ABP-labeled GCaseMut1-H8 were found in the liver 3 hours after infusion. The relative distribution of ABP-labeled GCaseMut1-H8 upon a 2-min unilateral ICV injection is shown in FIG. 12.

BD Assessed Through Enzyme Activity (4MU/βGlc Substrate)

To reproduce the results obtained using ABP-labelled GCase with a technique relying on non-labelled GCase, the 4MUβGlc enzymatic assay was used (essentially as described in Example 5). 4MUβGlc is a substrate that is not specific for GCase; other β-glucosidases present in tissues may possibly also convert it. To specifically quantify GCase activity, the homogenates were incubated with and without Conduritol B Epoxide (CBE), a GCase-specific inhibitor. GCase activity was then expressed as CBE-inhibitable 4MUβGlc activity.

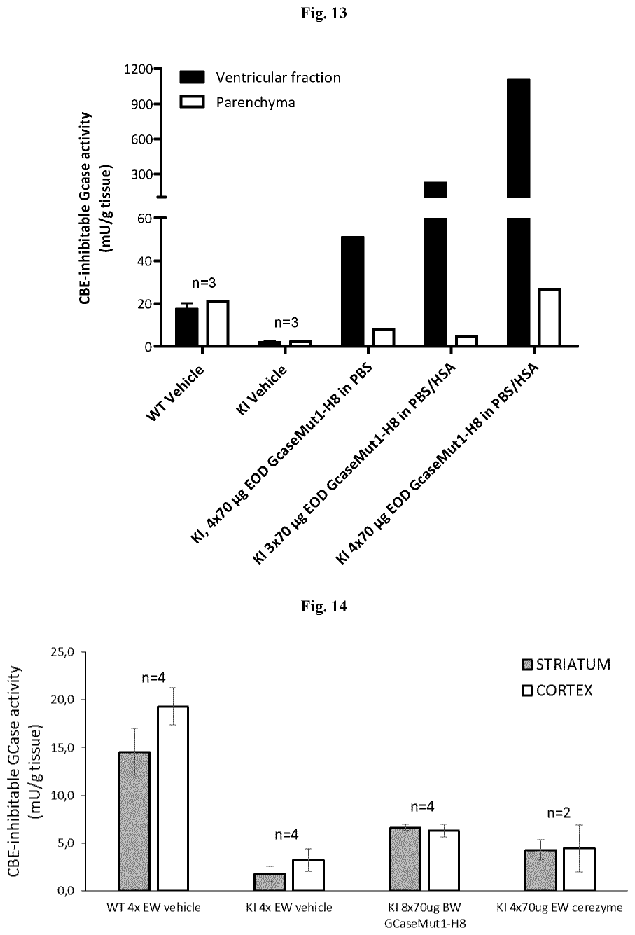

In a first experiment, GCase activity was determined in homogenized brain regions, more specifically the area around the ventricles versus parenchyma devoid of ventricles, 3 hours after the last of 4 every other day (EOD) unilateral ICV infusions with 70 μg GCaseMut1-H8 (FIG. 13). This confirmed the ABP results: 3 hours after infusion the highest amount of GCase activity could be found around the ventricles, ranging from 3 to 45 times the WT levels. Immunostaining suggests that this activity originated both from intra- and extracellular GCase. 3 hours after the 4th EOD treatment with 70 μg or 1400 mU GCaseMut1-H8 (average specific activity of 20 mU/μg), the total amount of GCase activity present in the brain tissue devoid of ventricles ranged from 3 to 10 mU (calculated with a brain volume of 400 mg). This corresponded to a 0.3-0.7% injected dose which was in the same range as determined via ABP labelling.

Over time, the distribution became more uniform throughout the brain. 48 hours after the last of 4 or 8 infusions with 70 μg GCaseMut1-H8, similar GCase activity levels were present in the cortex (no ventricles) and the striatum (containing ventricular regions) (FIG. 14). The endogenous GCase activity was slightly higher in the cortex compared to the striatum, both in Gba1 D409V KI and WT mice. Taken together, this suggested that slightly less GCase was distributed to the more distant cortex compared to the striatum, localised immediately adjacent to the ventricles.

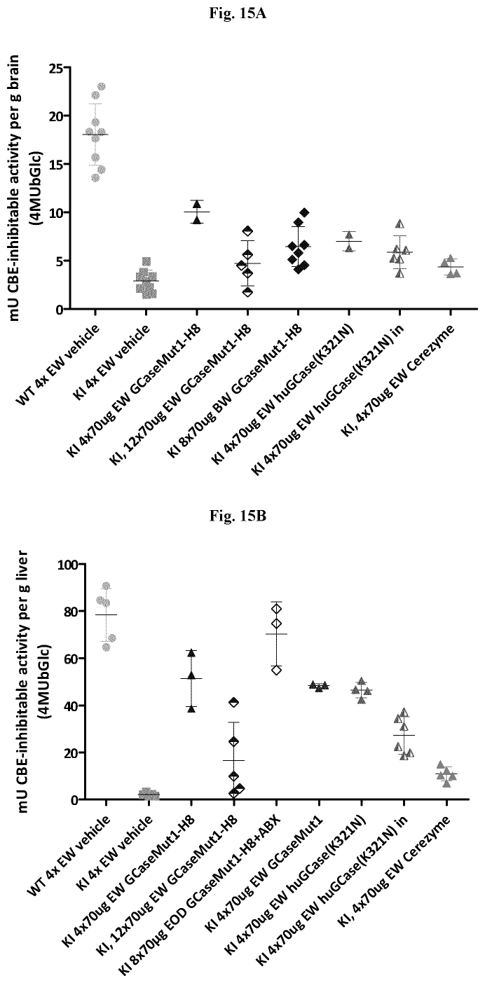

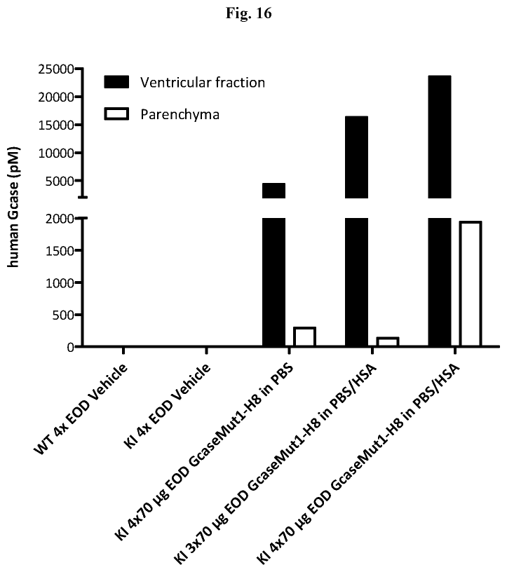

In several studies, GCase activity was also determined in homogenized left or right brain hemispheres and in liver tissue 48 hours after the last infusion (FIGS. 15A and B, respectively).

There was a similar increase in GCase activity in the left versus the right hemisphere upon treatment with either the OxyGCase variants or Cerezyme®, confirming that there was an equal distribution from the injected to the contralateral side (FIG. 10).

In WT mice, GCase activity was higher in the liver compared to the brain. However, Gba1 D409V KI mice displayed significantly lower GCase activity levels and this to the same extent in both organs. As a result, the % residual activity in KI brain (15% versus WT brain) was higher than in KI liver (3% versus WT liver). Upon repetitive ICV injections with OxyGCase variants, relatively more active GCase could be detected in the liver compared to the brain. 48 hours after the last of 4 weekly infusions with 70 μg GCaseMut1-H8, for example, there was a 25-fold increase in activity compared to untreated KI mice in the liver compared to a 3,5-fold increase in the brain. However, because the therapeutic window between WT and KI levels was smaller in brain than in liver, the % activity compared to WT was only slightly lower in brain (˜50%) than liver (˜75%).

These data were used to calculate the % injected dose: 48 hours after the last injection with 70 μg or 1400 mU GCaseMut1-H8 (average specific activity of 20 mU/μg), there was approximately 4 mU of GCase activity present in the brain (calculating with 0.4 g), and approximately 90 mU in the liver (calculating with 1.75 g). These values corresponded to 0.3% and 6% of the injected dose, respectively. A higher % injected dose in liver compared to brain was also observed 3 hours after infusion with ABP-labelled GCaseMut1-H8 (see above). More importantly, once GCase was taken up in brain tissue it appeared to be relatively stable as the % injected GCaseMut1-H8 dose was similar between 3 hours and 48 hours after the 4th treatment (ranging from 0.3-0.7% to 0.3%).

Compared to OxyGCase, ICV delivered Cerezyme® performed significantly worse in terms of increasing GCase activity, displaying only marginal improvements in the brain (3.5-fold of KI levels for GCaseMut1-H8 versus 1.5-fold of KI levels for Cerezyme®, both determined 48 hours after the last of 4 weekly ICV injections with 70 μg, FIG. 15A) and the liver (25-fold of KI levels for GCaseMut1-H8 versus 5-fold of KI levels for Cerezyme®, both determined 48 hours after the last of 4 weekly ICV injections with 70 μg, FIG. 15B). Note that the cellular uptake of OxyGCase was significantly higher compared to Cerezyme® (see FIG. 5). The substantial difference in the liver may in addition be potentially attributed to lower stability of Cerezyme® in circulation compared to OxyGCase.

Drug exposure went down in the liver as well as in the brain when prolonging the ICV treatment with 70 μg GCaseMut1-H8 from 1 to 3 months, which was likely due to an anti-drug antibody (ADA) response (see Example 11). The decrease in drug exposure in the brain was somewhat unexpected, taking into account that only a small percentage of antibodies in the blood crosses the blood-brain barrier. However, it is known that upon activation by an antigen, both T- and B-lymphocytes can enter the brain.

The activity levels reached in the brain were not that different when the injection was performed via a slow infusion (3 h) or a bolus injection (10-15 min). In contrast, less GCase reached the liver upon slow infusion instead of a bolus injection, which could be related to the different rate of GCase release in circulation (see FIG. 7 and FIG. 8).

BD Assessed Through Human GCase alphaLISA

Biodistribution as determined by activity measurement (see FIG. 13) was also validated with an alphaLISA to determine human-specific GCase protein levels (FIG. 16). Similar conclusions could be drawn from both methods.

### Example 10—Efficacy and Pharmacodynamics of the Recombinant Human Glucocerebrosidase (GCase) Polypeptides in Mice

As mentioned previously, accumulation of GCase substrates glucosylsphingosine (GlcSph) and glucosylceramide (GlcCer) is an important cause of pathological symptoms in Gaucher patients. The Gba D409V KI mice accumulate GlcSph, but not GlcCer, in the brain and peripheral organs such as the liver. The superior therapeutic potential of ICV injected OxyGCase variants for treating Gaucher compared to Cerezyme® was demonstrated by assessing the reduction of GlcSph levels in brain and liver.

Reduction of GlcSph in Whole Brain Hemisphere

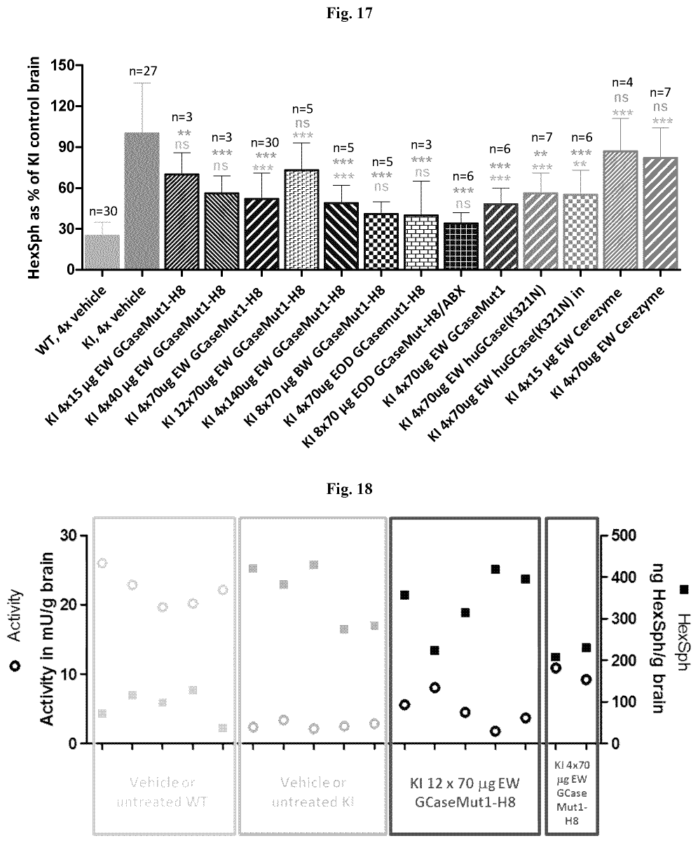

A summary of the substrate reduction results in the brain is presented in FIG. 17.

The HexSph levels consist of the two epimers, GlcSph and Galactosylsphingosine (GalSph), of which only GlcSph is a substrate for GCase. We demonstrated that the HexSph levels in WT mice only represented GalSph and that this GalSph level was identical in WT, treated and untreated Gba1 D409V KI mice (not shown).

All OxyGCase variants outperformed Cerezyme® when administered at an identical dose and regimen. 4 weekly bolus ICV treatments with 70 μg of GCaseMut1-H8, GCaseMut1 or huGCase(K321N) resulted in a statistically significant reduction of HexSph levels compared to vehicle-treated KI mice, while this was not the case for Cerezyme®. Hence, despite the detection of GCase activity in the brain upon 4 weekly ICV treatments with 70 μg of Cerezyme® (FIG. 15A), substrate levels did not decrease significantly. Considering that low levels of OxyGCase (around 200 ng in the full brain, cf. 0.3% of 70 μg) could effectively reduce substrate in the brain, a potential explanation for the discrepancy in observed Cerezyme® activity versus substrate reducing capacity is that the protein (and thus its activity) is present substantially only in cell types that do not accumulate GlcSph.

There was a good dose response observed up to 70 μg when comparing weekly treatments with 15 μg, 40 μg, 70 μg and 140 μg GCaseMut1-H8. Based on these results, the optimal dose for weekly OxyGCase treatment was set at 70 μg. When translated to non-human primates (NHP) and child patients based on brain weights, the 70 μg dose could correspond to a weekly dose of 10.5 mg for NHPs (60 g brain weight) and 210 mg for 2- to 3-year old children (approximately 1.2 kg brain weight).

Increasing the regimen from weekly to bi-weekly to every other day treatment further improved substrate reduction efficiency (not shown).

There was no observable difference in terms of efficacy and stability for the different OxyGCase variants (GCase(H145L/K321N)-His8, GCase(H145L/K321N), or GCase(K321N)) described in Example 1.

We already showed above via activity measurements that drug exposure decreased in brain when the ICV treatment was prolonged from 1 to 3 months. This was further substantiated with the GlcSph results. FIG. 18 shows the existence of a correlation between GCase activity and substrate levels in the different animals. The drug exposure decrease is likely due to an immune response against the GCase enzyme.

Reduction of GlcSph in Different Brain Regions

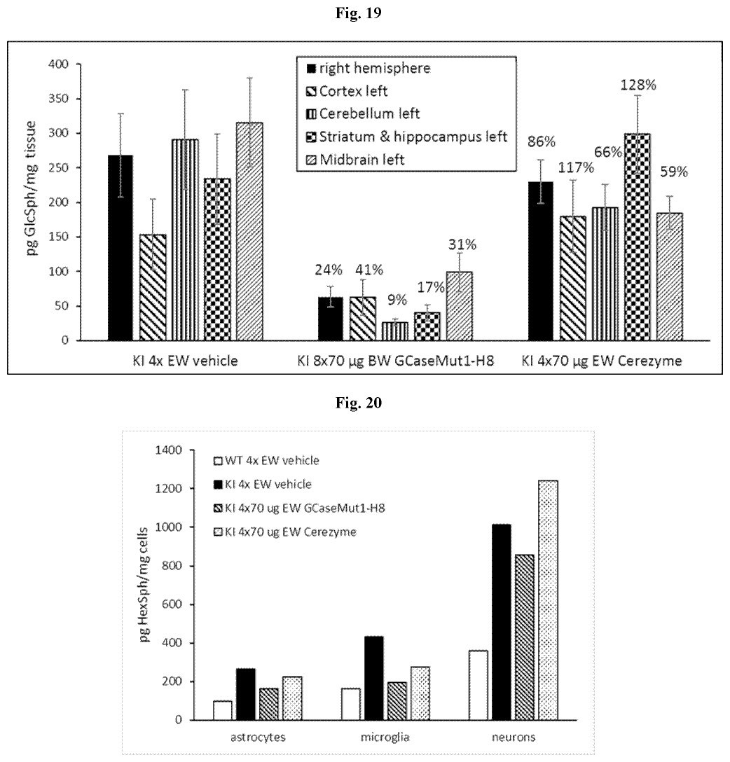

The cortex, cerebellum, striatum/hippocampus and midbrain were separated and HexSph levels determined. Results, expressed as GlcSph levels (by subtracting the WT HexSph (=GalSph) levels), from each region are shown in FIG. 19.

Substrate levels were efficiently reduced in all brain regions, and GlcSph accumulation was slightly region dependent with the lowest accumulation present in the cortex. This corresponds to a higher GCase activity in that region (see FIG. 14). Substrate reduction upon 8 bi-weekly treatments with GCaseMut1-H8 occurred in all regions, to a higher extend in striatum, hippocampus and cerebellum compared to midbrain and cortex. This is again in line with the enzyme levels measured through activity. Importantly, 4 weekly ICV infusions with 70 μg of Cerezyme® did not or only slightly reduced GlcSph in the analyzed regions. The region dependency of substrate reduction seemed different between OxyGCase and Cerezyme®. This may potentially be explained by differences in the cellular composition of the analyzed regions.

Reduction of GlcSph in Different Sorted Brain Cells

Single cells were prepared from brain hemispheres using a combination of the GentleMACS Octo Dissociator with heaters (Miltenyi Biotec, #130-096-427) and the Adult Brain Dissociation kit (Miltenyi Biotec, #130-107-677) for dissociation of rodent neural tissue older than P7 and subsequent isolation of neurons, astrocytes, or oligodendrocytes. Isolation of the astrocyte population, including the cell dissociation step, the debris removal step, the magnetic labelling using the astrocyte-specific Anti-ACSA-2 microbeads (Miltenyi Biotec, #130-097-678) and the magnetic separation were essentially done as described by the manufacturer (https://www.miltenyibiotec.com/upload/assets/IM0016290.PDF). The purity of the obtained astrocyte fraction was further increased by performing a second magnetic separation onto the positive cell fraction. The unlabelled cells obtained during the above procedures (microglia, neurons, oligodendrocytes and endothelial cells) were further processed to allow magnetic removal of the microglia using the Anti-CD11b MicroBeads (Miltenyi Biotec, #130-093-634), essentially as described by the manufacturer (https://www.miltenyibiotec.com/upload/assets/IM0016891.PDF). The unlabelled cellular fraction obtained after isolation of both the astrocytes and microglia mainly contained neuronal cells. For both magnetic separation steps, LS column (Miltenyi Biotec, #130-042-401) and a corresponding suitable QuadroMACS Separator (Miltenyi Biotec, #130-090-976) were used.

In summary, the astrocytes and microglia were positively selected via specific antibodies (Anti-ACSA-2 resp. anti-CD11b), while the neuronal cells were obtained via depletion of the previously mentioned cell types obtaining a neuron-enriched population. The HexSph levels, essentially determined as described in Example 7, in the neuronal fraction were higher than in astrocytes and microglia (FIG. 20). Gba1 D409V KI mice showed an upregulation of HexSph in all cellular fractions. Treatment of the mice with 4 weekly ICV injections of 70 μg GCaseMut1-H8 resulted in a reduction of accumulated HexSph in all three cell types, while a similar treatment regimen with Cerezyme® only reduced substrate in the microglia. These results further underscore the superiority of ICV OxyGCase compared to Cerezyme®.

Reduction of GlcSph in Liver

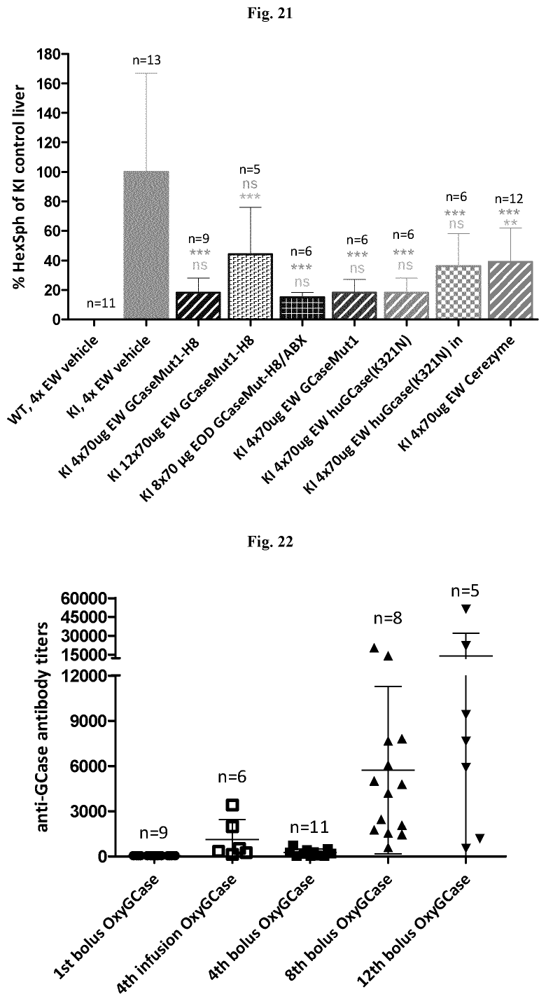

The impact of repetitive ICV treatments with OxyGCase variants compared to Cerezyme® on substrate levels in the liver was assessed (FIG. 21).

The accumulation of substrate was higher and more variable in the liver than in the brain (see FIG. 21 and FIG. 17), which can again be linked to the measured GCase activity levels (FIGS. 15A and 15B). Four weekly ICV treatments with 70 μg of different OxyGcase variants reduced substrate very efficiently, almost reaching WT levels (no statistically significant differences between OxyGCase-treated groups and WT controls, FIG. 21). This might explain why increasing the regimen (e.g. 4×70 μg EW GCasemut1-H8 versus 8×70 μg EOD GCasemut1-H8/ABX in FIG. 21) does not further improve efficiency in the liver. In contrast to what was observed in the brain, a slow infusion results in less efficient substrate reduction in the liver compared to a bolus injection. Taken together, these results demonstrate that ICV injected OxyGCase has therapeutic potential to treat the somatic symptoms in Gaucher disease patients, such as particularly in neuronopathic Gaucher disease patients.

Although four weekly ICV treatments with 70 μg Cerezyme® only slightly increased the activity in the liver, it did reduce substrate, although less efficiently than OxyGCase. This may be because Cerezyme® is solely taken up by Kupfer cells in a mannose-receptor-mediated way. As a result, GlcSph reduction by Cerezyme® would be restricted to a limited cell population in liver tissue.

### Example 11—Immune Response Induced by the Recombinant Human Glucocerebrosidase (GCase) Polypeptides in Mice

Anti-drug antibody (ADA) development is a common feature of systemic enzyme replacement therapy (ERT) for lysosomal storage diseases (LSD) (Harmatz. Enzyme Replacement Therapies and Immunogenicity in Lysosomal Storage Diseases: Is There a Pattern? Clin Ther. 2015, vol. 37, 2130-4). Since a significant amount of ICV-delivered OxyGCase enters the circulation, we assessed whether this induced an immune response in the mice.

The anti-drug antibody assay used for this purpose was an ELISA-based assay using purified OxyGCase (at a concentration of 2 μg/mL in 1×PBS) as coating agent. 100 μL of the coating solution was added per well and incubated overnight at 4° C. The next day, the coating agent was discarded and the wells of the ELISA plate were washed 3 times with 1×PBS+0.05% tween 20. Assay buffer (1×PBS+1% BSA) was added to the wells followed by a 1 hour incubation at 37° C. After discarding the assay buffer, 100 μL of diluted (plasma) sample (in 1×PBS+1% BSA) is added per well and incubated for 2 hours at 37° C. Upon discarding the samples, the wells of the plate were washed 3 times with 1×PBS+0.05% tween 20. After the washing step, the detection antibody (Horseradish Peroxidase-conjugated goat anti-mouse antibody, Sigma, # A4416) was diluted 10.000 times in assay buffer and 100 μL is added to each well. The plate was again incubated for 1 hour at 37° C., followed by removing the detection antibody solution and by washing the well 3 times with 1×PBS+0.05% tween 20. In a next step, 100 μL of ready-to-use TMB (tetramethylbenzidine) (Invitrogen, #002023) was added and the plate was incubated in the dark for 20 minutes at room temperature. The TMB was hydrolyzed by the peroxidase, generating a color compound which was proportional to the amount of anti-GCase antibodies present in the plasma. The reaction was stopped by adding 50 μL of 0.5 M sulphuric acid per well. The absorbance at 450 nm was measured within 15 minutes after addition of the sulphiric acid.

As can be observed in FIG. 22, anti-GCase antibodies were already present after 4 weekly ICV treatments with 70 μg OxyGCase, and their level further increased when the treatment was prolonged up to 2 or 3 months. The immune response varied significantly between the individual mice, which correlated with the variable GCase activity and HexSph levels in brain and liver upon long-term treatment, as described above.

OxyGCase contains sugar structures that are foreign to the mice and can thus cause an immune response. To determine whether the anti-drug antibodies are directed against the GCase enzyme or rather against the N-glycans, an ADA assay was developed using another lysosomal enzyme with identical sugar structures as OxyGCase. Only 1 out of the 13 mice that contained anti-GCase antibodies was reactive against that enzyme, albeit with a ˜100-fold lower titer. Therefore, we can conclude that the GCase enzyme rather than the N-glycans were antigenic in mice.

### Example 12—Histopathology in Mice Administered with Recombinant Human Glucocerebrosidase (GCase) Polypeptides

Several organs (liver, brain, spleen, kidney, lung and heart) were collected for histopathological analysis from untreated, vehicle- and OxyGCase-treated WT and Gba1 D409V KI mice. Cannulation and intracerebroventricular injection resulted in mild encephalitis and/or meningitis, both in WT and Gba1 D409V KI mice, and was independent of the injected substance. The inflammation in the brain was similar in vehicle- and OxyGCase-treated animals. Histopathology did not reveal any OxyGCase-related toxicity.

### Example 13—Intravenous (IV) Delivery of the Recombinant Human Glucocerebrosidase (GCase) Polypeptides in Mice

To assess the therapeutic potential of intravenously (IV) delivered OxyGCase in Gaucher patients, several pre-clinical studies were performed in wild-type (WT) and Gba1 D409V knock-in (KI) mice (see Example 6) to evaluate the biodistribution (BD), pharmacokinetics (PK), pharmacodynamics (PD) and efficacy upon IV injection(s) of different OxyGCase variants in comparison to its commercial counterpart, Cerezyme®. These studies are set forth in Examples 14-16.

Briefly, WT or Gba1 D409V KI mice were treated weekly (EW) with a bolus IV of test article for 1 to 4 consecutive weeks. In some of the studies, plasma was collected at different time points after IV treatment. One day after the last injection, mice were anaesthetized and blood was collected, followed by saline perfusion and dissection of peripheral organs like liver, spleen, heart and lung, and of brain. The samples were analyzed for hexosylsphingosine (HexSph) levels by RP-LC Q-TOF-MS (see Example 7), and for GCase levels by 4MUβGlc activity assay (see Example 5).

### Example 14—Plasma Pharmacokinetics (PK) of the Recombinant Human Glucocerebrosidase (GCase) Polypeptides in Mice

The circulation half-life (T_{1/2}) of active huGCase(K321N) was evaluated by IV injection of 60 U/kg of the enzyme into WT mice. Over a 24-hour period upon IV administration, different blood samples were collected from the tail vein, from which buffered plasma (pH 7.4) was prepared for activity analysis. The resulting GCase concentration-time curves allowed to calculate the circulation half-life for huGCase(K321N) using GraphPad Prism. Overall, the tested OxyGCase had a very short half-life of about 6 minutes (5.6±1.8 min).

### Example 15—Biodistribution of the Recombinant Human Glucocerebrosidase (GCase) Polypeptides in Mice Assessed Through Enzyme Activity (Using the 4MUβGlc Substrate)

Biodistribution of active GCase upon systemic (IV) treatment with OxyGCase variants was determined with the 4MUβGlc assay in liver (FIG. 24). 4MUβGlc is however a synthetic substrate that is not specific for GCase only, meaning that other β-glucosidases present in tissues might also hydrolyze it. To specifically quantify GCase activity, the homogenates were incubated with and without conduritol-b-epoxide (CBE), a GCase-specific inhibitor. GCase activity was then expressed as CBE-inhibitable 4MUβGlc activity.

GCase activity in the liver of Gba1 D409V KI mice was 3%±1% of the WT level. Weekly IV administration with 30 U/kg huGCase or huGCase(K321N) resulted in an increase in activity towards 28%±10% respectively 33%±11% of WT levels, 24 h after the last treatment. Importantly, an identical IV dose-regimen with the commercial counterpart, Cerezyme®, only resulted in an increase to 16%±4% of WT GCase activity level. Without wishing to be limited by any hypothesis or theory, the mannose-6-phosphate-mediated uptake of the GCase variants embodying the principles of the present invention by liver hepatocytes might explain the higher GCase activity in the liver when compared to Cerezyme®, which is only taken up by the macrophages. From this set of results it further appeared that the higher stability in circulation (i.e. physiological conditions) of huGCase(K321N) versus huGCase (and Cerezyme®) did not have a significant impact on the amount of active GCase that reached the liver cells, since the measured GCase activity was similar upon huGCase and huGCase(K321N) treatment. When the huGCase(K32IN) dose was increased to 300 U/kg, WT GCase activity levels were almost reached in the liver (93%±23% of WT level).

### Example 16—Efficacy and Pharmacodynamics of the Recombinant Human Glucocerebrosidase (GCase) Polypeptides in Mice

The accumulation of GCase substrates, glucosylsphingosine (GlcSph) and glucosylceramide (GlcCer), represents an important cause of pathological symptoms in Gaucher patients. The Gba1 D409V KI mice accumulate GlcSph, but not GlcCer, in the brain and peripheral organs such as the liver, spleen and heart. To assess whether IV injected OxyGCase variants possess a superior therapeutic potential for treating Gaucher disease compared to the commercial counterpart Cerezyme®, the reduction of GlcSph levels was determined in liver, spleen, heart and lung.

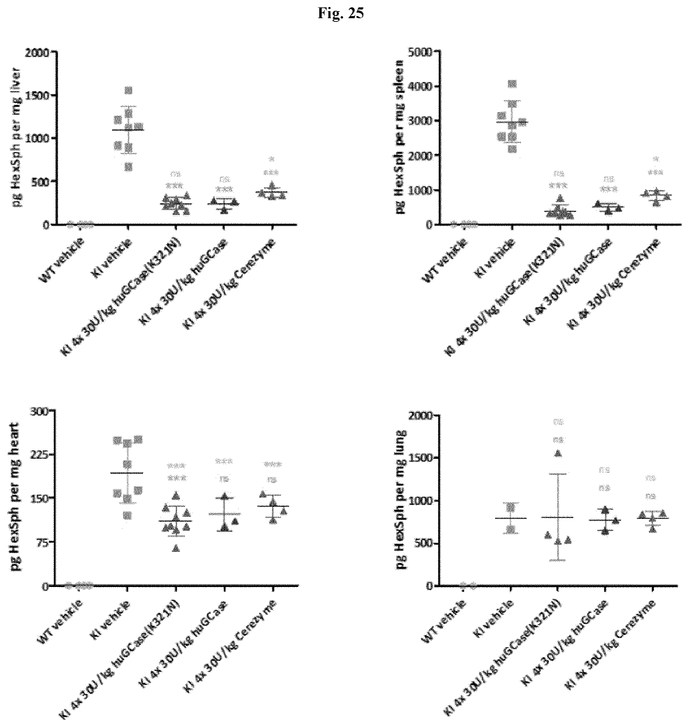

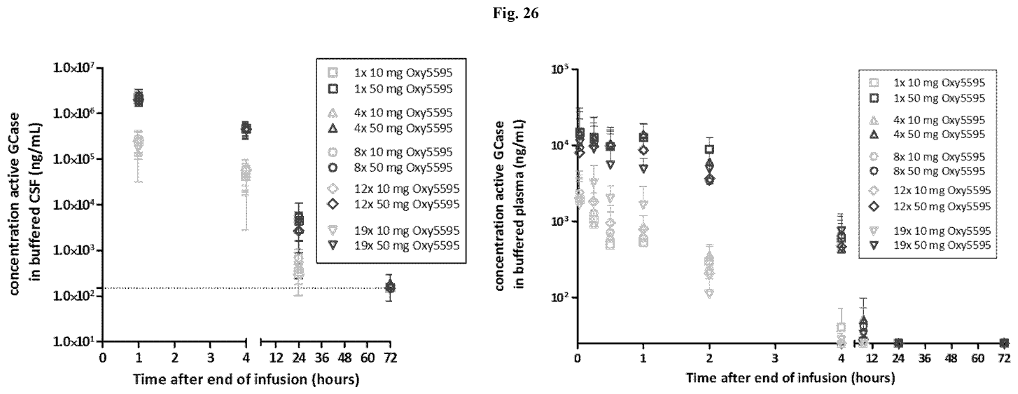

The higher GCase activity levels observed in the liver of KI mice that were IV treated with 30 U/kg huGCase or huGCase(K321N) compared to Cerezyme®, also translated in a better substrate reduction efficacy for the OxyGCase variants compared to their commercial counterpart (FIG. 25). Similar to the liver, IV administration of the OxyGCase variants reduced the substrate levels in the spleen more efficiently than did Cerezyme® when provided at the same dose-regimen. In the heart, there was no statistically significant difference in substrate reduction between the OxyGCase variants and Cerezyme®, although there seemed to be a trend that the huGCase(K321N) variant was better performant within the executed short-term study. In contrast, none of the 3 GCase variants were able to reduce the HexSph substrate that accumulates in the lung of Gba1 D409V KI mice, at least not after 4 weekly IV injections with a dose of 30 U/kg.

Examples 13-16 thus demonstrate, based on HexSph levels within different peripheral organs upon 4 weekly IV injections with 30 U/kg huGCase, huGCase(K321N) and Cerezyme, that huGCase, either with or without the K321N mutation, performed at least as good or better than the current standard of care for type 1 Gaucher patients. This dose regimen corresponds to the current therapeutic dose of Cerezyme in patients (60 U/kg every other week).

### Example 17—Toxicity Study of the Recombinant Human Glucocerebrosidase (GCase) Polypeptides in Cynomolgus Monkeys

Study Design

Thirty juvenile Cynomolgus monkeys, 15 months of age at first dosing (15 male, 15 female), representative of pediatric patients of both sexes, were surgically implanted with an ICV catheter in the left lateral ventricle for dose administration. Twenty-six animals were placed on study. Animals received 2.1 mL of Oxy5595 (huGCase(K321N) as described in previous examples) or vehicle (artificial cerebrospinal fluid, aCSF, pH 6.6) by ICV infusion once every week for a total of 23 doses. The study design is presented in Table 2. The low dose of 10 mg dosing was extrapolated from the therapeutic dose in mice (70 μg), based on the difference in brain size (0.4 g mouse brain weight versus 55-60 g non-human primate (NHP) brain weight). The five times therapeutic dose (50 mg) is the maximum that can be ICV administered due to limitations in Oxy5595 solubility and in infusion volume.

TABLE 2
Study design of the GLP non-human primate (NHP) toxicity study
3 Month ICV Study Design with Recovery

| | | | | Number of Animals | |
|---|---|---|---|---|---|
| | Test | Dose | Dose Conc. | per Necropsy Interval | |
| Group | Article | (mg) | (mg/mL) | Day 157 | Day 169 |
| 1 | Vehicle | 0 | 0 | 3M, 3F | 2M, 2F |
| | (aCSF)^{a} | | | | |
| 2 | Oxy5595 | 10 | 4.5 | 3M, 3F | — |
| 3 | Oxy5595 | 50 | 22.6 | 3M, 3F | 2M, 2F |

^{a}= artificial Cerebrospinal fluid
M= male,
F= female

In-life observations and measurements included body weight, food consumption, clinical observations, neurological and physical examinations, ophthalmology, electrocardiology, blood pressure, toxicokinetic and immunogenicity sampling, and clinical pathology evaluations. An IT catheter was installed in the lumbar spine for CSF sampling to study CSF Oxy5595 kinetics. Approximately 48 hours or 14 days after the final dose (recovery group), the animals were euthanized, and selected tissues harvested for biodistribution and/or histopathological evaluation.

In-Life Results

There were no Oxy5595-related clinical signs. There were no changes in body weight, food consumption, physical and neurological examinations, electrocardiography, ophthalmology, or organ weights. Weekly dose administration of Oxy5595 resulted in increased eosinophil numbers in both CSF and blood in a variable but consistent manner. However, after completion of dose administration, the numbers returned to near normal values within two weeks. There were no additional Oxy5595-related changes in the clinical pathology parameters observed.

Active GCase Concentration-Time Curves in CSF and Plasma

At five different occasions throughout the study (dose 1, 4, 8, 12 and 19), plasma (prior to dosing, and 2 min, 15 min, 30 min, 1 h, 2 h, 4 h, 8 h, 24 h and 72 h post dose) and CSF (prior to dosing, and 1 h, 4 h, 24 h, and 72 h post dose) were collected for GCase activity measurement with a validated assay. GCase activity was extrapolated to ng active GCase protein per mL based on a GCase standard curve, to establish the concentration-time profiles (FIG. 26). PK analysis was performed according to GLP guidelines using non-compartmental analysis in Pheonix® WinNonLin® version 6.3 software.

Results for CSF (FIG. 26, left panel): Quantifiable GCase levels were measured up to 24 h post injection in CSF. The earliest time point for CSF collection was 1 h after the end of infusion, which represents the highest measured active GCase concentration. At 72 hours post dosing, active GCase concentrations were below the limit of quantification (LLOQ), but still detectable. GCase level profiles in CSF were comparable after single and multiple administrations for both dose groups. The 5-fold increase in dose resulted in a slightly higher than proportional increase in exposure (Cmax and AUClast) for combined sexes with a range between 6.3 to 10.5 fold. The Oxy5595 exposure in CSF, in terms of AUClast, was several thousand folds higher compared to plasma for both dose groups. The concentration of active GCase in CSF at the 10 mg dose reached the Kuptake of Oxy5595 in neuronal cells (178±22 ng/mL) around 2 days post injection, independent of the number of treatments. No consistent sex-related differences in CSF PK parameters were observed.

Results for plasma (FIG. 26, right panel): The highest GCase levels were measured immediately after infusion (2 min) declining to levels around the LLOQ at 8 hours post dose. Although plasma concentration profiles varied significantly between animals, the profiles of both dose groups did not seem to alter drastically after single and multiple administrations. The 5-fold increase in dose resulted in similar to slightly higher than proportional increase in exposure (Cmax and AUClast) for combined sexes with a range between 3.5 to 18.8 fold. There were no consistent sex-related differences in plasma PK.

Brain Distribution of the GCase

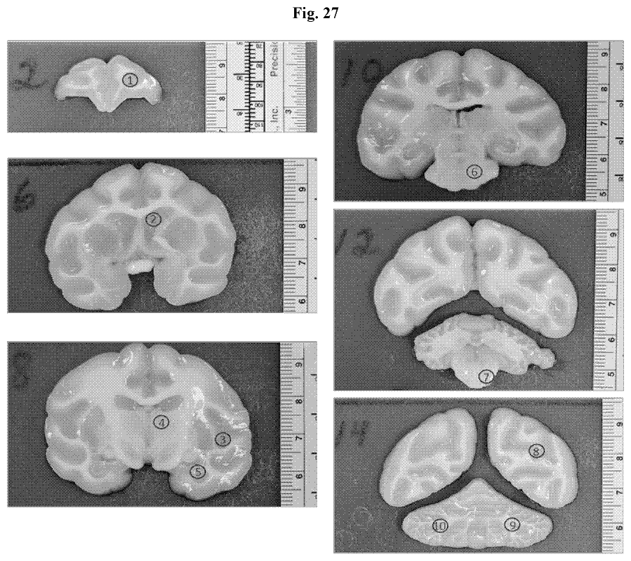

Two days after the last (23th) ICV treatment, animals were sacrificed for organ collection. Following perfusion, brains were harvested and sliced coronally into 3 mm broad slabs (approximately 17 slices per animal). The first slice and every other slice thereafter was fixed in neutral buffered formalin for histological analysis (see section ‘Histopathology’ below). From the second slice and every other slice thereafter, specimens were collected from various brain regions for test article activity analysis (GLP-compliant validated assay using the synthetic GCase substrate, 4MUβGlc). The regions selected for analysis (FIG. 27) were mainly regions that have been described to be affected in neuronopathic Gaucher disease patients: cerebral cortex, cerebellum, brain stem (pons and medulla oblongata), thalamus and corpus striatum (Maloney and Cumings. J. Neurol. Neurosurg. Psychiat. 1960, vol. 23, 207; Nilsson and Svennerhorn. Journal of Neurochemistry 1982, vol. 39, 709-718; Orvisky et al. Molecular Genetics and Metabolism 2002, vol. 76, 262-270; Perruca et al. Neuroradiology 2018, vol. 60, 1353-1356; Bremova-Ertl et al. Front Neurol. 2018, vol. 15, 711; Kaye et al. Ann Neurol. 1986, vol. 20, 223-30). The hippocampus was recently published to have a relatively high GCase expression in NHPs and was therefore also analyzed (Dopeso-Reyes et al. Brain Struct Funct. 2018, vol. 223, 343-355).

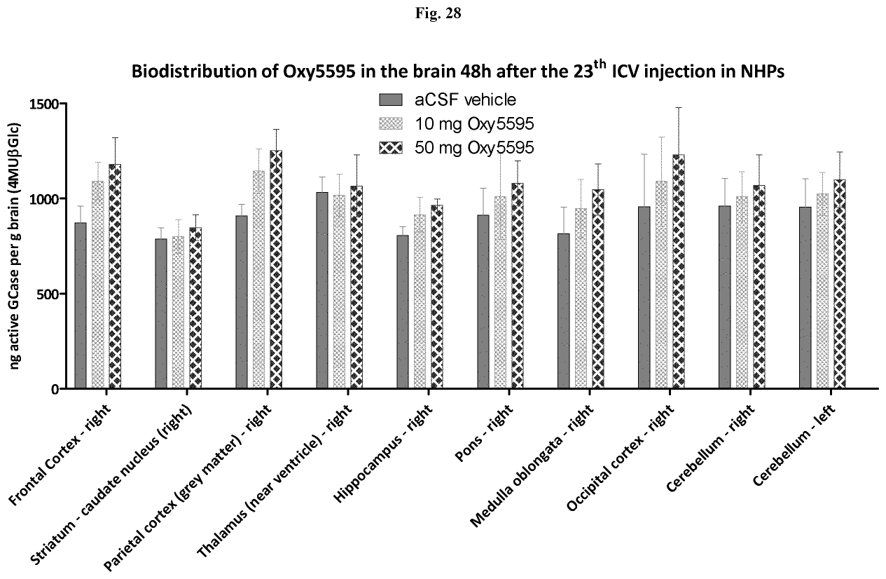

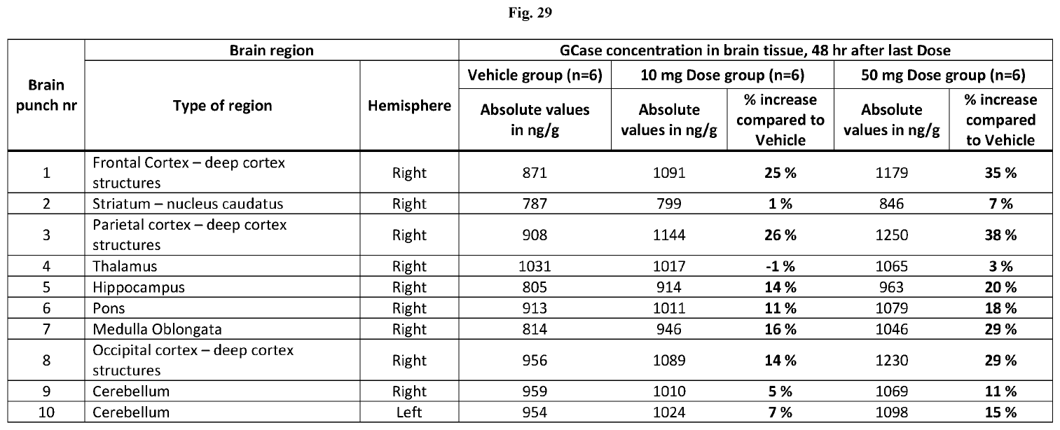

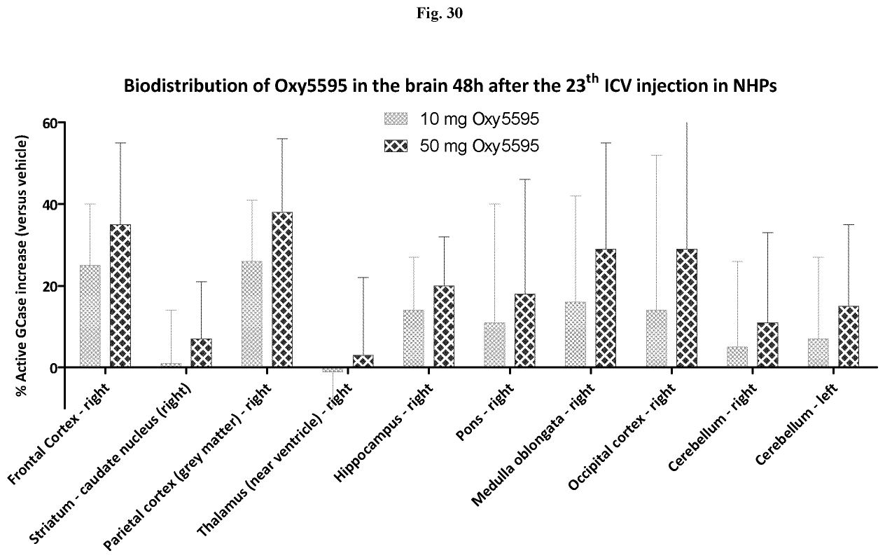

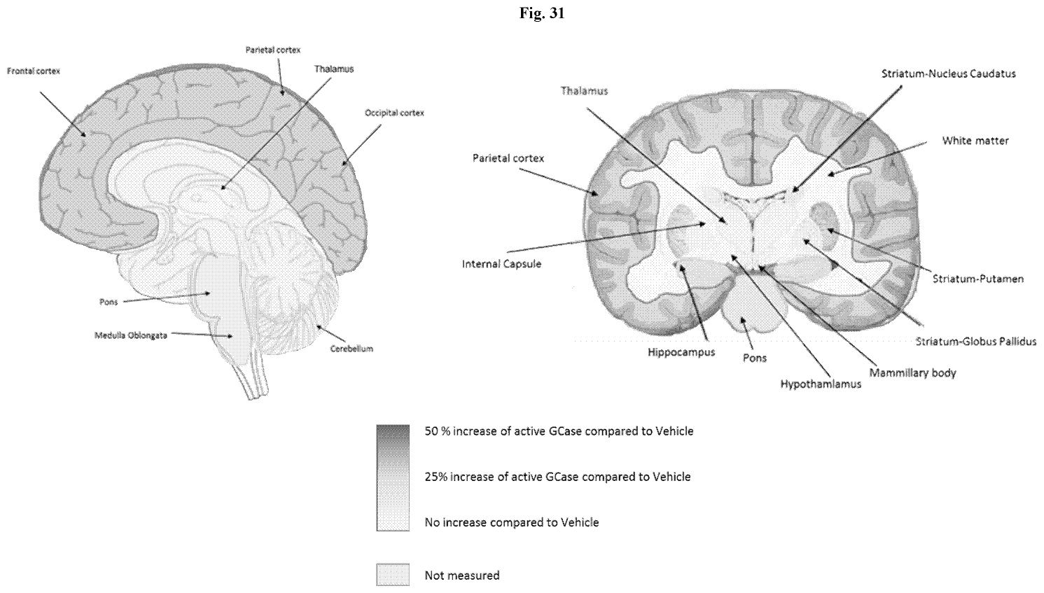

FIGS. 28 and 29 show that GCase activity was relatively homogenously present throughout the different brain regions of vehicle-treated WT animals. Unilateral ICV treatment with 10 mg or 50 mg Oxy5595 resulted in an equal distribution of GCase activity to both brain hemispheres as evidenced by the similar active GCase levels in left and right cerebellum. This observation corroborates our previously obtained results in mice, where ICV-administered Oxy5595 was also uniformly distributed over both hemispheres. GCase activity was detected in multiple regions of the brain, with the highest levels present in the deep layers of the frontal and parietal neocortex. A slightly lower GCase activity is observed in the hippocampus, pons, medulla oblongata and occipital cortex, followed by the cerebellum showing a moderate increase in GCase activity. The nucleus caudatus (striatum) and the thalamus showed no significant increase in GCase activity. In the positive areas, 50 mg Oxy5595 resulted in a 1.8±0.3-fold higher increase in GCase activity compared to 10 mg Oxy5595 (FIG. 30). A schematic representation of the distribution of active GCase throughout the brain upon Oxy5595 treatment can be found in FIG. 31.

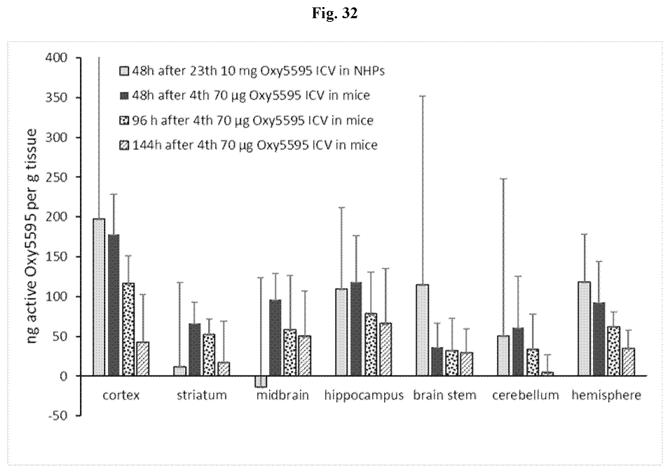

In mice, GCase activity increased from ˜10% of WT levels in untreated Gba1-deficient (KI) animals to ˜30% of WT levels in the left and the right hemisphere of Oxy5595-treated KI mice (4 weekly administrations of 70 μg). This 20% increase in mice was also observed with a corresponding dose of 10 mg Oxy5595 in NHPs (dose extrapolation based on brain volume) (FIG. 29). As can be observed in FIG. 32, the Oxy5595 levels per g tissue in mice and NHPs 48 h after Oxy5595 ICV treatment were similar in cortex, hippocampus and cerebellum, but slightly higher in the brain stem of NHPs compared to mice. The mouse striatum and midbrain accumulated Oxy5595, while this did not seem to be the case in NHPs, although in the latter, only a subregion was analyzed (nucleus caudatus and thalamus, respectively). Importantly, GCase activity remained above vehicle-treated levels in most mouse brain regions up to 6 days after ICV infusion (FIG. 32).

Repetitive ICV treatment with 70 μg Oxy5595 in Gba1-deficient mice resulted in a 3-fold reduction of the accumulated GCase substrate, GlucosylSphingosine, in the brain (measured 48 h after the last dose) compared to vehicle-treated mice. Since this dose resulted in similar GCase levels in mice and monkeys, this dose should also be sufficiently effective in reducing GCase substrate in the human brain, especially when taken into account that most of the neuronopathic Gaucher patients still have residual GCase activity to a varying extent.

Immunogenicity

Serum and CSF were collected prior to dose 1, 2, 5, 9, 13 and 20, and at necropsy, to determine the presence of antibodies specific for Oxy5595 using a validated screening assay. The cut point to distinguish positive from negative samples was set with a 95% confidence interval meaning that 5% of the samples would screen false positive. The results indicate that none of the vehicle-treated animals developed anti-GCase antibodies, while Oxy5595 treatment induced an antibody response in all but one animal before the 5th dose in serum and before the 9th dose in CSF. In some animals, the antibody response seemed to be transient. The intensity of the immunogenic response varied between animals but did not seem to be dose related, and followed the same trend in serum and in CSF (Table 3 and Table 4).

TABLE 3
Serum ADA response against Oxy5595
as determined by a screening assay.

| | | | 10 | 50 |
|---|---|---|---|---|
| RU | | vehicle | mg | mg |
| high | at least one | 0/10 | 3/6 | 1/10 |
| | value >30000 | | | |
| mid | at least one | 0/10 | 1/6 | 5/10 |
| | value >10000 | | | |
| low | all values <10000 | 0/10 | 2/6 | 4/10 |
| no | below cutpoint | 10/10 | 0/6 | 0/10 |
| | (998-1080) | | | |
| transient | last 2 points are lower | NA | 3/6 | 7/10 |
| | than the previous one | | | |

| | | | |
|---|---|---|---|
| positive at dose 5 | NA | 5/6 | 10/10 |

TABLE 4
CSF ADA response against Oxy5595 as
determined by a screening assay.

| | | | 10 | 50 |
|---|---|---|---|---|
| RU | | vehicle | mg | mg |
| high | at least one | 0/10 | 3/6 | 1/10 |
| | value >2000 | | | |
| mid | at least one | 0/10 | 2/6 | 4/10 |
| | value >500 | | | |
| low | all values <500 | 0/10 | 1/6 | 5/10 |
| no | below cutpoint | 10/10 | 0/6 | 0/10 |
| | (75-88) | | | |
| transient | last 2 points are lower | NA | 2/5 | 2/9 |
| | than the previous one | | | |

| | | | |
|---|---|---|---|
| positive at dose 5 | NA | 3/5 | 5/9 |
| positive at dose 9 | NA | 5/5 | 7/8 |

Other recombinant GCase enzymes (Cerezyme®, VPRIV®, Taliglucerase®) also induce an antibody response in Cynomolgus monkeys after repetitive systemic injections. Although some Gaucher patients develop an immunogenic response, it has generally not been associated with reduction of clinical response to treatment on established efficacy parameters (Rosenberg et al. Blood 1999, vol. 93, 2081-2088; Starzyk et al. Molecular Genetics and Metabolism 2007, vol. 90, 157-163; Pastores et al. Blood Cells, Molecules and Diseases 2016, vol. 59, 37-43; Zimran et al. Orphanet J Rare Dis. 2018, vol. 13, 36).

The ADA response in animal models is poorly predictive for the response in patients and therefore, the CHMP guidelines state that “while non-clinical studies aimed at predicting immunogenicity in humans are normally not required, animal models may be of value in evaluating the consequences of an immune response.” In our study, the presence of anti-Oxy5595 antibodies did not cause any clinical signs, nor did it have a major impact on the exposure in CSF or plasma.

Histopathology

The brain, spinal cord, spinal nerve roots, sensory ganglia (dorsal root ganglia/trigeminal ganglion), peripheral nerves, eyes with optic nerves, and non-nervous system tissues were examined using paraffin embedded sections and hematoxylin and eosin staining. In addition, brain sections were stained for astrocyte and microglial reactions.

There were some complications associated with the in vivo experimental procedures, like necrosis, microgliosis and astrocytosis around the catheter track in the brain. This is relatively common in studies utilizing direct delivery to the brain, but do not necessarily cause safety issues in the clinical trials.

A dose-related, general increase of cellular infiltrates, mainly composed of eosinophils, was observed in the brain, spinal cord, spinal nerve roots, sensory ganglia (dorsal root and trigeminal) and their surrounding tissues (meninges/epineurium) at both dose levels of Oxy5595. After the 2-week recovery period, the overall severity of the infiltrates was reduced in the highest dose group (no recovery mid-dose animals). The gross and microscopic findings were consistent with the interpretation that the 50 mg Oxy5595 was considered a “no observed adverse effect level” given the infiltrates did not appear to cause any damage to neurons or elicit a specific glial response. In the absence of clinical signs or other indications of adversity, these infiltrates appeared to be tolerated by the test animals, even at the highest dose tested.

Conclusions

In conclusion, twenty-three weekly ICV infusions with 10 mg or 50 mg Oxy5595 (formulated in aCSF, pH 6.6) over approximately 40 minutes was well tolerated in juvenile cynomolgus monkeys. The increase in eosinophils in CSF and blood returned to near normal values within two weeks after completion of dose administration. Eosinophilia can be indicative of a drug-related allergic reaction, which mostly occurs without clinical consequences, but could also be caused by the ICV device. Indeed, CSF eosinophilia is a relatively common finding in patients with ventricular shunts. A dose-related, general increase of cellular infiltrates, mainly composed of eosinophils, was observed in the CNS at both dose levels of Oxy5595, but did not appear to cause any damage to neurons or elicit a specific glial response. The infiltration ameliorated during the 2-week recovery period. Although ICV-administered Oxy5595 induced an immunogenic response in Cynomolgus monkeys, it did not seem to have an impact on drug exposure in CSF and circulation, nor did it cause any clinical signs. ICV-administered Oxy5595 was distributed throughout the brain tissue, including the areas that have been described to be involved in neuronopathic Gaucher disease, in amounts that were shown to efficiently reduce substrate in mice.

### Example 18

The following illustrates certain embodiments of GCase compositions and treatment regiments in accordance with the principles of the present invention:

A two-year old subject with Gaucher disease type 2 is treated weekly with 210 mg OxyGCase K321N formulated in artificial CSF pH 6.6 (+/−10 mL volume per 1h infusion dose) delivered by catheter implanted to the left ventricle.

A three-year old subject with Gaucher disease type 2 is treated weekly with 210 mg OxyGCase H145L/K321N formulated in artificial CSF pH 6.6 (+/−10 mL volume per 1h infusion dose) delivered by catheter implanted to the right ventricle.

A three-year old subject with Gaucher disease type 3 is treated weekly with 210 mg OxyGCase H145L/K321N formulated in artificial CSF pH 6.6 (+/−10 mL volume per 1h infusion dose) delivered by catheter implanted to the right ventricle.

A two-year old subject with Gaucher disease type 3 is treated weekly with 210 mg OxyGCase K321N formulated in artificial CSF pH 6.6 (+/−10 mL volume per 1h infusion dose) delivered by catheter implanted to the left ventricle.

An adult subject with Gba1-associated Parkinson's disease is treated weekly with 250 mg OxyGCase K321N formulated in artificial CSF pH 6.6 (+/−10 mL volume per 1h infusion dose) delivered by catheter implanted to the right ventricle.

A two-year old subject with Gaucher disease type 1 is treated weekly with 30 U/kg OxyGCase K321N formulated at 40 units/mL in 50 mM sodium citrate pH 5.5 delivered intravenously by bolus injection.

A three-year old subject with Gaucher disease type 1 is treated weekly with 30 U/kg OxyGCase H145L/K321N formulated at 40 units/mL in 50 mM sodium citrate pH 5.5 delivered intravenously by infusion.

A two-year old subject with Gaucher disease type 1 is treated biweekly with 60 U/kg OxyGCase K321N, lyophilised and reconstituted at 40 units/mL in 50 mM sodium citrate pH 5.5, delivered intravenously by bolus injection.

An adult subject with Gaucher disease type 1 is treated biweekly with 60 U/kg OxyGCase H145L/K321N, lyophilised and reconstituted at 40 units/mL in 50 mM sodium citrate pH 5.5, delivered intravenously by infusion.

1. A glucocerebrosidase preparation or a composition comprising glucocerebrosidase, wherein at least 30% of glycans comprised by the glucocerebrosidase comprise at least one mannose-6-phosphate moiety.

2. The preparation or composition according to claim 1, wherein at least 40%, or at least 50%, or at least 60%, or at least 70%, or at least 80%, or at least 90%, or at least 95%, or at least 98%, or at least 99%, or substantially all of the glycans comprised by the glucocerebrosidase comprise at least one mannose-6-phosphate moiety.

3. The preparation or composition according to claim 1 or 2, wherein at least some of the mannose-6-phosphate moiety-comprising glycans comprise two mannose-6-phosphate moieties, such as wherein at least 5%, or at least 10%, or at least 15%, or at least 20%, or at least 25%, or at least 30%, or at least 35%, or at least 40%, or at least 45% of the mannose-6-phosphate moiety-comprising glycans comprise two mannose-6-phosphate moieties.

4. A preparation or composition comprising glucocerebrosidase, wherein at least 10% of glycans comprised by the glucocerebrosidase comprise two mannose-6-phosphate moieties.

5. The preparation or composition according to claim 4, wherein at least 15%, or at least 20%, or at least 25%, or at least 30%, or at least 35%, or at least 40%, or at least 45% of the glycans comprised by the glucocerebrosidase comprise two mannose-6-phosphate moieties.

6. The preparation or composition according to any one of claims 1 to 5, wherein at least 40% of the glucocerebrosidase molecules are glycosylated, such as wherein at least 50%, or at least 60%, or at least 70%, or at least 80%, or at least 90%, or at least 95%, or at least 98%, or at least 99%, or substantially all of the glucocerebrosidase molecules are glycosylated.

7. The preparation or composition according to any one of claims 1 to 6, wherein the glucocerebrosidase is human wild-type glucocerebrosidase, or variant of human wild-type glucocerebrosidase having increased stability and/or specificity relative to human wild-type glucocerebrosidase.

8. The preparation or composition according to claim 7, wherein the glucocerebrosidase variant differs from human wild-type glucocerebrosidase by a single amino acid substitution at one or more positions selected from the group consisting of K321, H145, F316, and L317, such as preferably by a single amino acid substitution at K321, or at H145, or at K321 and H145, such as more preferably by K321N substitution, or by H145L substitution, or by K321N and H145L substitutions.

9. The preparation or composition according to any one of claims 1 to 8, wherein the mannose of the mannose-6-phosphate moiety is a terminal mannose.

10. The preparation or composition according to any one of claims 1 to 9, wherein the mannose-6-phosphate moiety-comprising glycans are each independently selected from the group comprising or consisting of PMan₅GlcNAc₂, PMan₄GlcNAc₂, PMan₃GlcNAc₂, P₂Man₆GlcNAc₂, and P₂Man₅GlcNAc₂.

11. The preparation or composition according to any one of claims 1 to 10, wherein the glucocerebrosidase is obtainable or obtained by uncapping and demannosylation of glucocerebrosidase recombinantly expressed by a fungal cell, such as a Yarrowia lipolytica cell, genetically engineered to produce glucocerebrosidase.

12. A pharmaceutical composition comprising the glucocerebrosidase preparation or composition according to any one of claims 1 to 11, optionally wherein:
the glucocerebrosidase is formulated with artificial cerebrospinal fluid (aCFS);
the pharmaceutical composition has pH of about 6.4 to 6.9, preferably of about 6.6; or
the glucocerebrosidase is formulated with aCFS and the pharmaceutical composition has pH of about 6.4 to 6.9, preferably of about 6.6.

13. The glucocerebrosidase preparation or composition according to any one of claims 1 to 11 or the pharmaceutical composition according to claim 12, for use in therapy.

14. The glucocerebrosidase preparation or composition according to any one of claims 1 to 11 or the pharmaceutical composition according to claim 12 for use in a method of treating a disease characterised by glucocerebrosidase deficiency.

15. The glucocerebrosidase preparation or composition or the pharmaceutical composition for use according to claim 14, wherein:
the disease is Gaucher disease;
the disease is non-neuronopathic Gaucher disease;
the disease is neuronopathic Gaucher disease;
the disease is neuronopathic Gaucher disease type 2 (GD2), type 3 (GD3), or perinatal lethal (GDPL);
the disease is glucocerebrosidase-associated alpha-synucleinopathy;
the disease is glucocerebrosidase-associated alpha-synucleinopathy selected from parkinsonism, Parkinson's disease, Multiple System Atrophy (MSA), or Lewis Body Dementia (LBD);
the preparation or composition or pharmaceutical composition is administered systemically;
the preparation or composition or pharmaceutical composition is administered intravenously (IV);
the preparation or composition or pharmaceutical composition is administered into the central nervous system;
the preparation or composition or pharmaceutical composition is administered intracerebroventricularly (ICV) or intrathecally;
the disease is neuronopathic Gaucher disease or glucocerebrosidase-associated alpha-synucleinopathy and the preparation or composition or pharmaceutical composition is administered intracerebroventricularly (ICV) or intrathecally administration; or
the disease is neuronopathic Gaucher disease or glucocerebrosidase-associated alpha-synucleinopathy and the preparation or composition or pharmaceutical composition is administered intracerebroventricularly (ICV).
